(19) United States
(12) Patent Application Publication
(10) Pub. No.: US 2025/0136910 A1
(43) Pub. Date: May 1, 2025

(54) DEVICES FOR USE AS BLOOD BRAIN BARRIER MODELS

(71) Applicants: Merck Sharp & Dohme LLC, Rahway, NJ (US); William Marsh Rice University, Houston, TX (US)

(72) Inventors: Nicole L. Buist, Holmdel, NJ (US); Martha J. Fowler, Kingwood, TX (US); Yufang He, Milltown, NJ (US); Anna Kristen Mai, Sugar Land, TX (US); Adam T. Procopio, Moorestown, NJ (US); Madison K. Royse, Houston, TX (US); Omid Veiseh, Bellaire, TX (US); Jun Xu, Jersey City, NJ (US)

(73) Assignees: Merck Sharp & Dohme LLC, Rahway, NJ (US); William Marsh Rice University, Houston, TX (US)

(21) Appl. No.: 18/917,316

(22) Filed: Oct. 16, 2024

Related U.S. Application Data
(60) Provisional application No. 63/592,982, filed on Oct. 25, 2023.

(51) Int. Cl. C12M 1/12; G01N 33/50

(52) U.S. Cl. CPC C12M 25/00; G01N 33/5058

(57) ABSTRACT

In some embodiments, devices for use as blood-brain barrier models are disclosed. The devices may include either a hydrogel component that surrounds a channel formed between a first opening and a second opening of the hydrogel component, or a hydrogel component that forms a groove. The devices may further include pericytes disposed in the channel or the groove, and endothelial cells may be disposed in the channel or the groove. The hydrogel component may include astrocytes (e.g., printed into the hydrogel component via projection stereolithography). Also disclosed are various methods of making and using these devices.

### CROSS-REFERENCE TO RELATED APPLICATIONS

This application claims the benefit of U.S. Provisional Application No. 63/592,982, filed Oct. 25, 2023; the entire contents of which are incorporated by reference herein.

### FIELD OF THE INVENTION

This invention related to devices for use as blood-brain barrier models. In particular, the invention relates to devices for use as blood-brain barrier models that include either a hydrogel component that surrounds a channel formed between a first opening and a second opening of the hydrogel component, or a hydrogel component that forms a groove and may further include pericytes disposed in the channel or the groove, and endothelial cells disposed in the channel or the groove.

### BACKGROUND

Despite recent technology advances in drug discovery, the success rate for neurotherapeutics remains alarmingly low compared to treatments for other areas of the body. The neurovascular unit (NVU) contains blood-brain barrier (BBB) capillaries, neurons, and supporting cells in the brain stroma (such as microglia, macrophages, and other glial cells) in addition to their basement membrane. One crucial part of the NVU is the BBB. The BBB is a structural and metabolic barrier that separates the bloodstream from the central nervous system. The BBB is responsible for protecting the brain and maintaining homeostasis in the central nervous system. The main cellular components of this barrier are non-fenestrated brain microvascular endothelial cells, pericytes, and astrocytes. The pericytes are in direct contact with the endothelial cells and ensheathe the endothelial lining. Astrocytes reside in the surrounding extracellular matrix and have end-feet projections that almost entirely encase the vessel of endothelial cells and pericytes. Each of these cells possesses unique features and plays an important role in the proper function of the BBB.

The endothelial cells within the BBB display unique properties from endothelial cells found throughout the rest of the body. The restrictive properties of the BBB are primarily attributed to the junctional complexes that are present between endothelial cells. These junctional complexes comprise tight junctions and adherens junctions, both of which work with the actin cytoskeleton and play a role in the formation and maintenance of cell-cell contacts that comprise a mature endothelial barrier. The adherens junctions are responsible for initiating, mediating, and maintaining cell-cell contacts. They perform these tasks via nectin-based adhesions, which are responsible for forming the first adhesions with neighboring cells, then cadherin-based adhesions, which maintain the cell-cell adhesion. VE-cadherin, p120-catenin, beta-catenin, and alpha-catenin are adherens junctions. VE-Cadherin is a transmembrane protein, while the catenins are located within the cytoplasm. The tight junctions are responsible for regulating paracellular transport involving the movement of ions and small molecules between endothelial cells. Occludin, the claudins (claudin 3, -5, and -12), the junctional adhesion molecules (JAM A, -B, and -C), and the zonula occludens (ZO-1, -2, -3) are tight junctions. The claudins are considered to be the principal proteins that determine the restrictiveness of the barrier for paracellular permeability. Occludin, the claudins, and the junctional adhesion molecules are transmembrane proteins, while the zonula occludens are localized to the cytoplasmic region of cells. Brain endothelial cells display higher expression of cell-cell junction proteins, creating a more restrictive barrier than endothelium in other parts of the body.

In addition to providing a more restrictive barrier via upregulation of cellular adhesion junctions, brain endothelial cells also display the presence of transport systems to facilitate maintenance of brain homeostasis. One type of transporter that plays a crucial role in the BBB is an efflux pump. Efflux pumps play a role in transporting lipophilic molecules out of brain endothelial cells and into circulation. While these pumps are essential for mitigating the effects of harmful compounds entering the brain, they also pose a challenge to delivery of drugs for neurological disorders. Thus, one strategy employed for drug delivery to the brain focuses on the inhibition of efflux pumps. Two of the most well-studied efflux transporters in the BBB include P-glycoprotein (P-gp/ABCB1) and breast cancer resistance protein (BCRP/ABCG2), which are both members of the ATP-binding cassette (ABC) transporter family. Since transport through the BBB is so restricted, another family of transporters exists to transport necessary compounds across the BBB that are not able to cross via paracellular or transcellular pathways. Paracellular transport in the BBB, which occurs in-between the endothelial cells, is limited due to the presence of tight junctions, so only small lipophilic molecules with a molecular weight less than 500 Daltons can use this route. Transcellular transport, which occurs through the endothelial cells, is also limited and generally restricted to small hydrophilic or lipophilic molecules, or gas molecules such as carbon dioxide and oxygen. High-weight molecules that have polar characteristics require transport via carrier-mediated transport. One well-reported type of carrier-mediated transport in the BBB is receptor-mediated transcytosis (RMT). In RMT, the receptors present on endothelial cells interact with compounds that have the necessary ligand in the blood, undergo endocytosis with that compound forming a vesicle that travels through the endothelial cell, and the contents of the vesicle can then be released into the brain parenchyma. Two well-studied pathways for RMT include the transferrin receptor (TfR), which is responsible for iron transport into the brain, and the low-density lipoprotein receptor related-protein 1 (LRP-1), which is responsible for the transport of numerous substances into and out of the brain.

Pericytes are mural cells that wrap around the endothelium and play a crucial role in regulation of the BBB via direct contact and through signaling pathways. Pericytes in the BBB are present on the abluminal side of the blood vessel wall and are embedded in the basement membrane between the endothelial cells and endfeet of surrounding astrocytes. These cells are present along blood vessels throughout the body; however, they have the highest density within the central nervous system, indicating that they play an important role in maintenance of the vasculature in the central nervous system. The ratio of pericytes to endothelial cells varies throughout tissues in the body, varying between 1:1 and 1:100; however, that ratio is reported to be highest in the central nervous system, ranging from 1:1 to 1:3. In the central nervous system, pericytes play a role in blood vessel formation via angiogenesis, development and maintenance of the BBB, operation of the neuroimmune network, and modulation of blood flow to the brain. In vitro experiments involving co-cultures of endothelial cells and pericytes have suggested that pericytes may play a role in increasing endothelial barrier function. One potential reason for this is through pericyte secretion of angiopoietin 1(b), a known anti-permeability factor.

Astrocytes are the most prevalent glial cell within the brain and are in contact with the abluminal side of blood vessel walls via projections of their endfeet. Astrocytes are known for their elongated, stellate morphology that have long projections radiating from the cell body, the terminal part of the projections are referred to as the astrocyte endfeet. The endfeet express specialized molecules and channels that play a role in regulating BBB ionic concentrations and protein transporters. Much like pericytes, astrocytes play a key role in the development and maintenance of the BBB. The role of astrocytes occurs through the production of secreted factors which enable the formation of strong tight junctions and proper interactions between endothelial cells and pericytes. These three cell types all play an indispensable role in the formation and maintenance of the BBB.

Drug delivery to the central nervous system to treat cancer or neurodegenerative diseases is a major challenge due to the presence of the BBB. While the BBB is essential for keeping harmful substances out of the brain, it also prevents the delivery of therapeutic agents and limits their effectiveness. The failure rate for new drugs targeting the central nervous system is high compared to drugs targeting other areas of the body. This high failure rate is attributed to three main factors: lack of understanding of the basic principles of central nervous system diseases, potential central nervous system side effects of the therapy, and the inability of drugs to cross the BBB. This difficulty in developing drugs for treatment of central nervous system diseases is exemplified when looking at Alzheimer's disease, one of the most prevalent neurodegenerative diseases. Between 2002-2012, the failure rate of Alzheimer's disease drug development was 99.6%, and the success rate has remained similarly low in the past decade. The BBB is considered to be the bottleneck in drug development for neurotherapeutics, as it restricts the transport of essentially 100% of large-molecule therapeutics (MW>500 Da), including peptides, recombinant proteins, monoclonal antibodies, RNA interference (RNAi)-based drugs and gene therapies, as well as the transport of >98% of small-molecule drugs. In order to improve the success rate of drug development for neuropharmaceuticals, there is a need for better models to investigate the mechanisms of transport across the BBB to inform the design of therapeutics and strategies, as well as evaluate drug candidates to take further down the pipeline in pharmaceutical development.

Historically, studies for drug development of neuropharmaceuticals has been assessed using both in vivo and in vitro methods. In vivo methods using animals are beneficial in recapitulating biological complexities and are generally regarded as the gold standard for preclinical validation of therapies in development; however, species differences between animal and human systems may produce results that fail to predict complications or proper efficacy in humans. It has been reported that more than 80% of candidate drugs that were successful in animal models failed in clinical trials. Additionally, animal models are costly and labor-intensive, and are facing increasing pressure due to the ethical issues associated with their use. As a result, there has been a shift towards developing more complex in vitro models that incorporate more biological intricacies and can better replicate the in vivo environment for better prediction of therapeutic outcomes.

The first in vitro technologies used relied on 2D cell culture for drug discovery. Models consisting of a single layer of cells grown in the lab are robust, reproducible, and easy to analyze. These models may be useful to assess the cytotoxicity of a drug candidate; however, they are often too simplistic to inform about more complicated mechanisms, such as assessing transport through the endothelial cells of the BBB. To enhance the complexity of the model, modifications have been made to the 2D culture system to enable drug transport studies.

One such modification is accomplished by culturing cells on a filter membrane that resides in a well and is referred to as the Transwell. The Transwell assay is one of the most common in vitro models used to assess barrier function, which is examined in this system via transendothelial electrical resistance (TEER) and molecular permeability. TEER involves the measurement of the electrical resistance between the apical and basolateral sides of monolayer-cultured endothelial cells to assess barrier tightness. TEER is mostly representative of the electrical resistance against paracellular transport (transport between cells). When the monolayer is packed tightly, there are less gaps for ions and other charged species to flow through, which results in a higher resistance. Permeability assays assess barrier function by measuring the fluorescence intensity of a fluorescently conjugated molecule permeating through monolayer-cultured cells from the apical to the basolateral side of a Transwell. These two methods are commonly used in tandem and can be highly valuable when assessing barrier function. This Transwell set-up has been adapted to become a BBB model through the incorporation of astrocytes and pericytes in culture with brain microvascular endothelial cells. Typically, the endothelial cells are cultured on the apical side of the membrane, while the pericytes and astrocytes are cultured in on the basolateral side of the Transwell. While this model is simple to fabricate, low cost, reproducible, high throughput, and allows for the incorporation of multiple cell types, it has several disadvantages. This model lacks necessary features of the microenvironment (cell-cell and cell-matrix interactions), shear stress, 3D cellular organization, the ability to concurrently examine cell morphology, and has technical complications such as inaccurate readings if endothelial cells grow non-uniformly and manual sampling which introduces human error.

Recapitulating complexities within a physiological setting, such as fluid shear stress, mechanical stimulation, and interactions with neighboring cells and the extracellular matrix is helpful in the development of an in vitro model that more closely mimics biologic complexities in vivo. These aspects play a key role in influencing, signaling pathways, stimuli response, and gene and protein expression, all of which are crucial in the prediction of human drug response. As a result, there has been a shift towards developing 3D in vitro technologies. One of the main technologies in this area is the organ-on-a-chip platform. Organs-on-chips are microfluidic in vitro cell culture systems that recapitulate the functions of human organs. These microfluidic devices are populated with relevant cell types in hollow 3D channels and can be cultured under perfusion for the formation of mature cell layers. These models are used in disease modeling and high throughput drug screening to assess the physiologic response of human tissues or organs. The first organ-on-a-chip fabricated demonstrated lung function with the incorporation of epithelial and endothelial cells, as well as cyclic mechanical strain and fluid flow. Since that breakthrough work was published in 2010, much effort has been focused on extending the organ-on-a-chip platform to replicate other organs of the body, and the most development has been seen in the areas of heart-on-a-chip, intestine-on-a-chip, kidney-on-a-chip, liver-on-a-chip, and lungs-on-a-chip. Given the challenges in drug discovery regarding neuropharmaceuticals, there has been significant interest in extending the organ-on-a-chip technology towards recapitulating the BBB for use as a screening tool. Microfluidics/organ-on-a-chip devices are now one of the most prevalent technologies to assess BBB function. One of the most used microfluidic BBB chips is the commercially available OrganoPlate® 3-lane 40 manufactured by Mimetas. In this platform, each chip consists of three adjacent channels—two perfusion channels and one gel channel (containing ECM). This platform does not use separation membranes between the channels but uses PhaseGuide™ technology that allows for membrane-free culture. The channels for this platform are 400 μm×220 μm (w×h). This platform has been adapted to create a BBB in this model. To do this, brain endothelial cells are perfused into one channel and grown against the extracellular matrix in the gel channel. On the other side of the gel channel, astrocytes and pericytes are added in to complete this BBB model. Using this strategy, the presence of tight and adherens junctions was confirmed, and function was demonstrated via dextran permeability and antibody transport. While the Mimetas microfluidic chip is high throughput, commercially available, enables shear stress, is generated from robust materials, and allows for planar 3D vessel fabrication, there are still disadvantages associated with this method. Perfusion in the OrganoPlate® occurs via rocking, providing oscillatory which may not be optimal for mimicking in vivo conditions. It has been reported that, contrary to steady flow, oscillatory flow has the potential to disrupt cell-cell junctions and fails to induce cytoskeletal remodeling typically associated with flow conditions. Additional limitations associated with microfluidics include a technically complex fabrication procedure that makes it difficult to iterate through various architectural designs, and lack of cell-cell contacts, which is helpful in recreating the BBB in an in vitro model.

While the two most common in vitro BBB models used are Transwell and microfluidic models, several other classes of models have been developed more recently and are currently being investigated for use as a new class of BBB models. Other classes of models include spheroids, self-assembled microvasculature, vessel-like structures in gels, and bioprinted models. It has been reported than human brain endothelial cells, pericytes, and astrocytes spontaneously form into multi-cellular spheroids and recreate the complex arrangement of these cells in a way that resembles the BBB. In fabrication of self-assembled vasculature, a cell-laden ECM gel is loaded into a microdevice, and brain endothelial cells undergo vasculogenesis and self-assemble with the pericytes and astrocytes into microvascular structures with all three cell types in close contact. These models provide a more accurate representation of the 3D in vivo environment where the cells are in direct contact and forming microvascular structures; however, there are challenges associated with the perfusion of spheroids and reproducibility due to heterogeneity of branching patterns in self-assembled microvasculature. Vessel-like structures in gel models are formed by introducing a microneedle or perfusion through an ECM-gel to form a hollow cylindrical channel. This method enables incorporation of perfusion and shear stress, has the potential to be used with more physiologic materials than microfluidics, and permits real-time visualization of cell morphology changes, however it is limited to simple geometries. Another group of BBB models is generated via bioprinting. In this group, two-photon lithography has been used to fabricate a microfluidic system with cylindrical, porous microcapillaries, and another group has demonstrated a PCL/PLGA microfluidic perfusion system fabricated by freeze-coating a 3D-printed sacrificial template. The use of 3D bioprinting for fabricating a BBB model holds immense potential for reducing the manufacturing difficulties associated with traditional microfluidics, enabling tunable perfusion and shear stress, permitting real-time visualization of cell morphology, and fabricating complex geometries that allow for the precise placement of cell types.

### SUMMARY OF THE DISCLOSURE

The devices disclosed herein leverage 3D bioprinting to fabricate a perfusable BBB model with anatomically relevant cell populations and organization that has potential for use as a scalable screening tool in drug development. As described in the Examples, a static hydrogel hemi-cylinder was first used to assess media selection, co-culture ratios, cell incorporation order, and culture durations in a resource-conservative and high-throughput manner. These parameters were used to fabricate a BBB model containing human brain endothelial cells, pericytes in direct contact on the abluminal side of the endothelium, and astrocytes in the surrounding bulk matrix. The disclosed BBB models demonstrated key cellular adhesion junctions as well as functionally relevant efflux transporters and receptor-mediated transporters. These parameters were extended to a 3D perfusable channel model to demonstrate the model's potential for assessing barrier function to various model compounds. The Examples discussed below leverage this BBB model with ultrasound to study the assisted transport of a therapeutically relevant molecule across the engineered BBB. In summary, this 3D perfusable BBB hydrogel model provides an in vitro platform to inform decisions in the design of neurotherapeutics and strategies employed to facilitate transport across the BBB.

In some embodiments, the invention provides a 3D printable hydrogel BBB model, which mimics the cellular composition and arrangement of the BBB with human brain endothelial cells lining the surface, pericytes in direct contact with the endothelial cells on the abluminal side of the endothelium, and astrocytes in the surrounding printed bulk matrix. In some examples, a simple, static printed hemi-cylinder model has been developed to determine design parameters such as media selection, co-culture ratios, and cell incorporation timing in a resource-conservative and high-throughput manner. Presence of cellular adhesion junction, VE-Cadherin, efflux transporters, P-gp and BCRP, and receptor-mediated transporters, TfR and low-density LRP-1 were confirmed via immunostaining demonstrating the ability of this model for screening in therapeutic strategies that rely on these transport systems. Design parameters determined in the hemi-cylinder model may be translated to a more complex, perfusable vessel model to demonstrate its utility for determining barrier function and assessing permeability to model therapeutic compounds. This 3D-printed BBB model uses projection stereolithography to fabricate a perfusable BBB model, enabling the patterning of complex vessel geometries and precise arrangement of cell populations. This model demonstrates potential as a platform to investigate the delivery of neurotherapeutic compounds and drug delivery strategies through the BBB, providing a useful scalable in vitro screening tool in central nervous system drug discovery and development.

In some embodiments, devices for use as a BBB model comprise (i) a hydrogel component defining a channel formed between a first opening and a second opening of the hydrogel component, wherein the hydrogel component comprises astrocytes, (ii) pericytes disposed in the channel, and (iii) endothelial cells disposed in the channel.

In some embodiments, the pericytes disposed in the channel adhere to the hydrogel component. In some embodiments, the pericytes adhere to the hydrogel component via the astrocytes. In some embodiments, the endothelial cells disposed in the channel adhere to the hydrogel component. In some embodiments, the endothelial cells adhere to the hydrogel component via the astrocytes. In some embodiments, the pericytes are in direct contact with the endothelial cells. In some embodiments, the pericytes are in direct contact with the endothelial cells on a side of the endothelial cells that faces the hydrogel component. In some embodiments, the astrocytes are human brain astrocytes. In some embodiments, the pericytes are human brain microvascular pericytes. In some embodiments, the endothelial cells are human brain microvascular endothelial cells.

In some embodiments, the hydrogel component further comprises poly(ethylene glycol) diacrylate (PEGDA). In some embodiments, the hydrogel component further comprises lithium phenyl-2,4,6-trimethylbenzoylphosphinate (LAP). In some embodiments, the hydrogel component further comprises gelatin methacrylate (GelMA). In some embodiments, the channel has a diameter of at least 200 micrometers and at most 2 millimeters. In some embodiments, the devices further comprise a housing chamber enclosing at least a portion of the hydrogel component, wherein the housing chamber has a first port aligned with the first opening and has a second port aligned with the second opening. In some embodiments, the housing chamber is made from a plastic.

In some aspects, devices for use as a BBB model comprise a hydrogel component that forms a groove, wherein the hydrogel component comprises astrocytes, pericytes disposed in the groove, and endothelial cells disposed in the groove.

In some embodiments, the pericytes disposed in the groove adhere to the hydrogel component. In some embodiments, the pericytes adhere to the hydrogel component via the astrocytes. In some embodiments, the endothelial cells disposed in the groove adhere to the hydrogel component. In some embodiments, the endothelial cells adhere to the hydrogel component via the astrocytes. In some embodiments, the pericytes are in direct contact with the endothelial cells. In some embodiments, the pericytes are in direct contact on a side of the endothelial cells that faces the hydrogel component. In some embodiments, the astrocytes are human brain astrocytes. In some embodiments, the pericytes are human brain microvascular pericytes. In some embodiments, the endothelial cells are human brain microvascular endothelial cells.

In some embodiments, the hydrogel component further comprises poly(ethylene glycol) diacrylate (PEGDA). In some embodiments, the hydrogel component further comprises lithium phenyl-2,4,6-trimethylbenzoylphosphinate (LAP). In some embodiments, the hydrogel component further comprises gelatin methacrylate (GelMA).

In some embodiments of any of the disclosed aspects, the endothelial cells express one or more BBB transporters. In some embodiments, the one or more BBB transporters comprise P-gp 1 efflux pump, BCRP efflux pump, transferrin receptor, or low-density lipoprotein receptor-related protein 1. In some embodiments, the one or more BBB transporters comprise p-gp 1 efflux pump, BCRP efflux pump, transferrin receptor, and low-density lipoprotein receptor-related protein 1.

In some aspects, methods of selecting a neurotherapeutic compound comprise introducing a plurality of neurotherapeutic compounds, either separately or collectively, to the channel of one or more of the devices described herein or to the groove of one or more of the devices described herein; determining the relative permeabilities of the plurality of neurotherapeutic compounds into the hydrogel component; and selecting at least one neurotherapeutic compound based on its determined relative permeability.

In some embodiments, the methods further comprise applying ultrasound to said one or more devices after introducing the plurality of neurotherapeutic compounds, either separately or collectively, to said channel or groove.

In some aspects, methods of making the hydrogel component of any of the disclosed devices comprises preparing a pre-hydrogel mixture that comprises poly(ethylene glycol) diacrylate (PEGDA), lithium phenyl-2,4,6-trimethylbenzoylphosphinate (LAP), and gelatin methacrylate (GelMA); suspending astrocytes in the pre-hydrogel mixture to obtain a hydrogel composition; and 3D printing the hydrogel composition using projection stereolithography to obtain the hydrogel component of the device.

In some embodiments, the hydrogel component comprises 2.25 to 4.25 wt % 3.4 kDa PEGDA, 7 to 27 mM LAP, and 5 to 15 wt % GelMA. In some embodiments, the astrocytes are at a concentration of at least 3 million cells per milliliter and at most 9 million cells per milliliter. In some embodiments, said 3D printing forms a groove on the hydrogel component. In some embodiments, said 3D printing forms a channel inside the hydrogel component.

In some aspects, methods of making any of the disclosed devices comprise making a hydrogel component with a channel using any of the disclosed applicable methods; introducing pericytes at a concentration of 20 to 40 million cells per milliliter into the channel; and introducing endothelial cells at a concentration of 20 to 40 million cells per milliliter into the channel.

In some embodiments, the endothelial cells are introduced into the channel after the pericytes.

In some aspects, methods of making any of the disclosed devices comprise making a hydrogel component with a groove using any of the disclosed applicable methods; introducing pericytes at a concentration of 0.2 to 0.6 million cells per milliliter into the groove; and introducing endothelial cells at a concentration of 0.2 to 0.6 million cells per milliliter into the groove.

In some aspects, methods of making any of the disclosed devices comprise providing a hydrogel component defining a receiving feature formed between a first opening and a second opening of the hydrogel component, wherein the hydrogel component comprises astrocytes; introducing pericytes in the receiving feature; and introducing endothelial cells in the receiving feature.

In certain embodiments of the methods of making any of the devices described herein, introducing pericytes in the receiving feature comprises adhering the pericytes to the hydrogel component. In certain embodiments, introducing endothelial cells in the receiving feature comprises adhering the endothelial cells to the hydrogel component. In certain embodiments, introducing pericytes comprises placing the pericytes in direct contact with the endothelial cells.

In certain embodiments of the methods of making any of the disclosed devices described herein, providing a hydrogel component comprises preparing a pre-hydrogel mixture that comprises poly(ethylene glycol) diacrylate (PEGDA), lithium phenyl-2,4,6-trimethylbenzoylphosphinate (LAP), and gelatin methacrylate (GelMA); suspending astrocytes in the pre-hydrogel mixture to obtain a hydrogel composition; and 3D printing the hydrogel composition using projection stereolithography to obtain the hydrogel component of the device.

In certain embodiments of the methods of making any of the devices described herein, 3D printing the hydrogel composition comprises forming a channel as the receiving feature. In certain embodiments, 3D printing the hydrogel composition comprises forming a groove as the receiving feature.

In certain embodiments of the methods of making any of the devices described herein, introducing pericytes comprises introducing pericytes at a concentration of 0.2 to 0.6 million cells per milliliter into the receiving feature. In certain embodiments, introducing endothelial cells comprises introducing endothelial cells at a concentration of 0.2 to 0.6 million cells per milliliter into the receiving feature.

In certain embodiments of the methods of making any of the devices described herein, the astrocytes are at a concentration of at least 3 million cells per milliliter and at most 9 million cells per milliliter. In certain embodiments, the astrocytes are human brain astrocytes.

In some embodiments, the endothelial cells are introduced into the groove after the pericytes.

### BRIEF DESCRIPTION OF THE DRAWINGS

FIGS. 1a-1c show GFP human brain microvascular endothelial cells (GFP HBMECs) after 3 days growth in a 24-well plate in Vasculife media (FIG. 1a), Human Brain Microvascular Endothelial Cell media (FIG. 1b), and Human Brain Microvascular Pericyte media (FIG. 1c). GFP HBMECs were plated at a density of 200 K cm⁻² and grown for 3 days in a 24-well plate.

FIGS. 2a-2c show GFP HBMECs immunostained for cell-cell junction, VE-Cadherin, after 3 days growth in a 24-well plate in Vasculife media, Human Brain Microvascular Endothelial Cell media, and Human Brain Microvascular Pericyte media. GFP HBMECs were plated at a density of 200 K cm⁻² and grown for 3 days in a 24-well plate, fixed, and immunostained for VE-Cadherin. Each well comprised a different media type, Vasculife media (FIG. 2a), HBMEC media (FIG. 2b), and HBMP (FIG. 2c) media. The top row shows a representative 10× image of each well, and the bottom row shows the magnified region of interest (ROI) that is selected from the 10× image (dashed box).

FIGS. 3a-3c show hydrogel 2.5D printed hemi-cylinder/half pipe model used for screening studies. FIG. 3a shows a rendering of the printed half pipe where the construct has dimensions of 8×8×2 mm. FIG. 3b shows a schematic of the interior contained portion of the half pipe without the wall extensions visualized. Cells are seeded in this inner region and morphology is examined in the curved pipe region as well as the top, flat region. FIG. 3c shows a top view of a printed half pipe seeded with endothelial cells shown on day 3. Many of the images shown to assess morphology are a 10× image of the pipe surface that enables visualization of cell coverage and morphology.

FIG. 4 shows endothelial monolayer progression on 2.5D printed half pipe. GFP HBMECs were seeded on the printed half pipe at a density of 400 K cm⁻² and cultured for three days with Vasculife media. The top row is a stitch showing the coverage across the entire pipe surface and the bottom row is a 10× image of the selected ROI shown in the top row (dashed box). Over the three days, the endothelial cells attach, spread, and form a confluent monolayer on the half pipe surface.

FIGS. 5a-5c show GFP HBMECs after 3 days growth on a printed 2.5D hydrogel in Vasculife media (FIG. 5a), Human Brain Microvascular Endothelial Cell media (FIG. 5b), and Human Brain Microvascular Pericyte media (FIG. 5c). GFP HBMECs were seeded at a density of 200 K cm⁻² and grown for 3 days. Each hydrogel half pipe utilized a different media type. The top row shows a representative 10× image of each well, and the bottom row shows the magnified ROI that is selected from the 10× image (dashed box).

FIGS. 6a-6c show GFP HBMECs immunostained for cell-cell junction, VE-Cadherin, after 3 days growth on a printed 2.5D hydrogel in Vasculife media (FIG. 6a), Human Brain Microvascular Endothelial Cell media (FIG. 6b), and Human Brain Microvascular Pericyte media (FIG. 6c). GFP HBMECs were seeded at a density of 200 K cm⁻² and grown for 3 days before being fixed and immunostained for VE-Cadherin. Each hydrogel half pipe utilized a different media type. The top row shows a representative 10× image of each well, and the bottom row shows the magnified ROI that is selected from the 10× image (dashed box). Gaps in the endothelial monolayer are indicated by the arrows.

FIGS. 7a-7d show simultaneous seeding co-culture of endothelial cells and pericytes on 2.5D printed half pipes. FIG. 7a shows a timeline for co-culture simultaneous seeding where endothelial cells and pericytes are mixed together and seeded on the half pipe surface on day 1 and cultured together until day 3. Endothelial cell seeding density was held constant at 400 K cm⁻² for all conditions, and pericyte concentration ranged from 40-400 K cm⁻² for the studies. FIG. 7b shows endothelial cell only control on days 0 and 3. FIG. 7c shows day 0 images taken after the four-hour seeding incubation period. Each ratio condition is one half pipe, starting with the highest concentration of pericytes on the left. The top column is a stitch of the half pipe for visualization of coverage across the entire pipe surface, and the bottom rows are 10× regions of a selected region of the pipe (dashed box), split into component views of the combined image, the GFP channel to visualize endothelial cell morphology, and the mCherry channel to visualize pericyte cell morphology. FIG. 7d Day 3 images after static culture for the half pipes with daily media changes using Vasculife media.

FIGS. 8a-8b show a sequential seeding co-culture timeline on 2.5D printed half pipes and pericyte morphology progression over 3 days of culture. FIG. 8a shows a timeline for co-culture sequential seeding where pericytes are seeded alone on day 0 and cultured for 3 days prior to the addition of endothelial cells. FIG. 8b shows pericyte morphology progression between days 0 and 3 prior to endothelial cell inclusion. The left-most columns are the highest concentrations of pericytes, and the concentration decreases with the progression of columns.

FIGS. 9a-9d show morphology progression of sequentially seeded co-culture of endothelial cells and pericytes on 2.5D printed half pipes. FIG. 9a shows pericytes only control on day 3 overall (day 0 HBMEC) and day 6 overall (day 3 HBMEC). Pericytes were seeded at 400 K cm⁻² on day 0 overall. FIG. 9b shows endothelial cell only control on day 3 overall (day 0 HBMEC) and day 6 overall (day 3 HBMEC). Endothelial cells were seeded at 400 K cm⁻² on day 3 overall. FIG. 9c shows endothelial cells were seeded onto the pericyte containing half pipes on day 3 overall, day 0 HBMEC. Endothelial cell density was kept constant across all four ratio conditions and the endothelial cell only control at 400 K cm⁻². FIG. 9d shows morphology of endothelial cells and pericytes on day 6 overall, day 3 HBMEC after daily media changes with Vasculife media.

FIGS. 10a-10c show endpoint morphology of sequentially seeded co-culture of endothelial cells and pericytes on 2.5D printed half pipes. FIG. 10a shows pericytes only control on day 9 overall (day 6 HBMEC) Pericytes were seeded at 400 K cm⁻² on day 0 overall. FIG. 10b shows endothelial cell only control on day 9 overall (day 6 HBMEC) Endothelial cells were seeded at 400 K cm⁻² on day 3 overall. FIG. 10c shows morphology of co-culture of endothelial cells and pericytes on day 9 overall, day 6 HBMEC, the final day of culture before endpoint assessment, after daily media changes with Vasculife media.

FIG. 11 shows confocal images of VE-Cadherin morphology for all ratio conditions of sequentially seeded co-culture of endothelial cells and pericytes on 2.5D printed half pipes. After a total of 9 days of cell culture on the 2.5D printed half pipes, the half pipes were fixed and immunostained for cell-cell junction, VE-Cadherin. The presence of this endothelial specific cell-cell junction is seen in all ratio conditions. The top row shows a single plane confocal image of the half pipe VE-Cadherin morphology and the bottom row shows a magnified selected ROI from the 10× image (dashed box).

FIGS. 12a-12c show astrocytes printed into 2.5D hydrogel half pipe. Human brain astrocytes (HBAs) incorporated into the hydrogel precursor print solution at a concentration of 6M mL⁻¹. Images shown were taken after allowing the astrocyte-containing half pipe to equilibrate in Vasculife media overnight after printing. FIG. 12a shows top view of the astrocyte-containing printed half pipe. Astrocytes are distributed uniformly throughout the length and width dimensions of the hydrogel half pipe. FIG. 12b shows magnified ROI of the box shown in (a) where the focus plane of the height dimension is in the bulk gel. In the bulk, the astrocytes appear to be more confined and balled up (dashed box). FIG. 12c shows magnified ROI of the box shown in (a) where the focus plane of the height dimension is on the curved surface of the half pipe. Along the half pipe surface, the astrocytes appear to be more elongated and spreading along the surface of the gel (dashed box).

FIGS. 13a-13g shows morphology progression of sequentially seeded tri-culture of endothelial cells and pericytes on astrocyte-containing 2.5D printed half pipes. FIG. 13a shows a timeline for tri-culture sequential seeding where astrocyte-containing half pipes are printed and allowed to soak overnight, pericytes are seeded on day 1 at a density of 400 K cm⁻² and cultured for three days, and endothelial cells are seeded on day 3 at a density of 400 K cm⁻². The construct is then cultured for an additional six days after the incorporation of endothelial cells, for a total tri-culture timeline of ten days. FIG. 13b shows morphology of astrocytes along surface of printed half pipe immediately after printing is very rounded. FIG. 13c shows morphology of astrocytes along surface of printed half pipe after overnight culture immediately before pericyte seeding demonstrates some astrocyte elongation. FIG. 13d shows morphology of pericytes after seeding at 400 K cm⁻² on day 3. FIG. 13e shows morphology of endothelial cells after seeding at 400 K cm⁻² on day 6. FIG. 13f shows morphology of tri-culture on day 6 and on day 9 (FIG. 13g).

FIGS. 14a-14c show cell-cell junction, VE-Cadherin, immunostaining on 2.5D hydrogel half pipe tri-culture model. FIG. 14a shows a side view of tri-culture hydrogel half pipe immunostained for VE-Cadherin on day 9 of culture. 3D renderings show GFP HBMECS, RFP HBMPs, and VE-Cadherin. FIG. 14b shows top view of tri-culture hydrogel half pipe immunostained for VE-Cadherin on day 9 of culture. FIG. 14c shows maximum intensity projections of a confocal z-stack going through the entire z-dimension of the half pipe from the base of the pipe to the top of the hemi-cylinder channel sides (z dimension ˜400 μm). Individual channels of the maximum intensity projections shown are GFP HBMEC (488 nm laser), RFP HBMP (560 nm laser), Hoechst (405 nm laser), and VE-Cadherin (647 nm laser).

FIGS. 15a-15c show pericyte morphology differences between mono-, co-, and tri-culture half pipe models shown on days 1, 3, 6, and 9 of culture for pericytes seeded at 400 K cm². FIG. 15a shows mono-culture pericyte morphology for pericytes seeded alone on an acellular half pipe. FIG. 15b shows co-culture pericyte morphology for pericytes seeded on an acellular half pipe with endothelial cells seeded on top (400 K cm⁻²) after the day 3 images. FIG. 15c shows tri-culture pericyte morphology for pericytes seeded on an astrocyte-containing half pipe (6M mL⁻¹) with endothelial cells seeded on top (400 K cm⁻²) after the day 3 images.

FIGS. 16a-16c shows endothelial morphology differences between mono-, co-, and tri-culture half pipe models shown on between days 1-6 of endothelial cell culture (days 4-9 overall culture) for endothelial cells seeded at 400 K cm⁻². FIG. 16a shows mono-culture endothelial morphology for endothelial cells seeded alone on an acellular half pipe. FIG. 16b shows co-culture endothelial morphology for endothelial cells seeded on an acellular half pipe that had pericytes seeded on top on day 1 (400 K cm⁻²). FIG. 16c shows tri-culture endothelial cell morphology for endothelial cells seeded on an astrocyte-containing half pipe (6M mL⁻¹) that had pericytes seeded on top on day 1 (400 K cm⁻²).

FIGS. 17a-17c show VE-Cadherin morphology differences between mono-, co-, and tri-culture half pipe models after immunostaining on day 9. FIG. 17a shows mono-culture VE-Cadherin morphology for endothelial cells seeded alone on an acellular half pipe. FIG. 17b shows co-culture VE-Cadherin morphology for endothelial cells seeded on an acellular half pipe that had pericytes seeded on top on day 1 (400 K cm⁻²). FIG. 17c shows tri-culture VE-Cadherin cell morphology for endothelial cells seeded on an astrocyte-containing half pipe (6M mL⁻¹) that had pericytes seeded on top on day 1 (400 K cm⁻²). The top row shows a single plane confocal image of the half pipe VE-Cadherin morphology and the bottom row shows a magnified selected ROI from the 10× image (dashed box).

FIGS. 18a-18d show cell layer coverage across half pipe surface for mono-, co-, and tri-culture conditions shown for culture days 4-9 overall (1-6 for the endothelial cells). FIG. 18a shows mono-culture of endothelial cells (seeding density 400 K cm⁻²) on acellular half pipe. FIG. 18b shows mono-culture of pericytes (seeding density 400 K cm⁻²) on acellular half pipes shown between days 4-9 overall. FIG. 18c shows co-culture of pericytes (seeding density 400 K cm⁻²) and endothelial cells (seeding density 400 K cm⁻²) seeded sequentially on acellular half pipes. FIG. 18d shows tri-culture of pericytes (seeding density 400 K cm⁻²) and endothelial cells (seeding density 400 K cm⁻²) seeded sequentially on astrocyte-containing half pipes (printed at 6M mL⁻¹).

FIGS. 19a-19c show expedited tri-culture timeline in 2.5D half pipes. FIG. 19a shows a timeline for tri-culture sequential seeding where astrocyte-containing half pipes are printed and allowed to soak overnight, pericytes are seeded on day 1 at a density of 400 K cm⁻² and cultured for only one day, and endothelial cells are seeded on day 1 at a density of 400 K cm⁻². The construct is then cultured for only three after the incorporation of endothelial cells, for a total tri-culture timeline of five days. FIG. 19b shows half pipe images showing the tri-culture hydrogel model after pericyte seeding on day 1. FIG. 19c shows half pipe images showing the tri-culture hydrogel model on day 4 after the endothelial cells have been cultured for three days on the half pipe. The entire half pipe is shown for assessment of cell layer retention, while the selected ROI (dashed box) is shown for assessment of cell morphology.

FIGS. 20a-20d show P-glycoprotein (P-gp) immunostaining on 2.5D hydrogel half pipe tri-culture model. FIG. 20a shows a side view of tri-culture hydrogel half pipe immunostained for P-gp on day 9 of culture. 3D renderings show GFP HBMECs, RFP HBMPs, and P-gp. FIG. 20b shows atop view of tri-culture hydrogel half pipe immunostained for P-gp on day 9 of culture (ten-day tri-culture timeline). FIGS. 20c and 20d show maximum intensity projections of a confocal z-stack going through the entire z-dimension of the half pipe from the base of the pipe to the top of the hemi-cylinder channel sides (z dimension ˜400 μm) with (20c) and without (20d) P-gp. Individual channels of the maximum intensity projections shown are GFP HBMEC (488 nm laser), RFP HBMP (560 nm laser), Hoechst (405 nm laser), and P-gp (647 nm laser).

FIGS. 21a-21d show Breast Cancer Resistance Protein (BCRP) immunostaining on 2.5D hydrogel half pipe tri-culture model. FIG. 21a shows side view of tri-culture hydrogel half pipe immunostained for P-gp on day 9 of culture. 3D renderings show GFP HBMECs, RFP HBMPs, and Breast cancer resistance protein. FIG. 21b shows a top view of tri-culture hydrogel half pipe immunostained for BCRP on day 9 of culture (ten-day tri-culture timeline). FIGS. 21c and 21d show maximum intensity projections of a confocal z-stack going through the entire z-dimension of the half pipe from the base of the pipe to the top of the hemi-cylinder channel sides (z dimension ˜400 μm) with (21c) and without (21d) BCRP. Individual channels of the maximum intensity projections shown are GFP HBMEC (488 nm laser), RFP HBMP (560 nm laser), Hoechst (405 nm laser), and BCRP (647 nm laser).

FIGS. 22a-22d show Transferrin (TfR) immunostaining on 2.5D hydrogel half pipe tri-culture model. FIG. 22a shows a side view of tri-culture hydrogel half pipe immunostained for TfR on day 9 of culture. 3D renderings show GFP HBMECs, RFP HBMPs, and TIR. FIG. 22b shows a top view of tri-culture hydrogel half pipe immunostained for P-gp on day 9 of culture (ten-day tri-culture timeline). FIGS. 22c and 22d show maximum intensity projections of a confocal z-stack going through the entire z-dimension of the half pipe from the base of the pipe to the top of the hemi-cylinder channel sides (z dimension ˜400 μm) with (22c) and without (22d) TfR. Individual channels of the maximum intensity projections shown are GFP HBMEC (488 nm laser), RFP HBMP (560 nm laser), Hoechst (405 nm laser), and TfR (647 nm laser).

FIGS. 23a-23d show low density lipoprotein receptor related protein 1 (LRP-1) immunostaining on 2.5D hydrogel half pipe tri-culture model. FIG. 23a shows a side view of tri-culture hydrogel half pipe immunostained for low density LRP-1 on day 9 of culture. 3D renderings show GFP HBMECs, RFP HBMPs, and Low density LRP-1. FIG. 23b shows a top view of tri-culture hydrogel half pipe immunostained for P-gp on day 9 of culture (ten-day tri-culture timeline). FIGS. 23c and 23d show maximum intensity projections of a confocal z-stack going through the entire z-dimension of the half pipe from the base of the pipe to the top of the hemi-cylinder channel sides (z dimension ˜400 μm) with (23c) and without (23d) LRP-1. Individual channels of the maximum intensity projections shown are GFP HBMEC (488 nm laser), RFP HBMP (560 nm laser), Hoechst (405 nm laser), and low density LRP-1 (647 nm laser).

FIG. 24 shows that VEGF removal media protocol does not result in confluent monolayer formation for HBMECs. GFP HBMECs seeded in vascular channel at 30M mL⁻¹ and perfused at 5 μL min⁻¹ for duration of the study. GFP HBMEC serpentine perfused with complete vasculife media containing VEGF for three days, then switched to a non-VEGF containing vasculife media for the next three days; perfused for a total of six days. Images shown on day 3, day 6, and after fixing and immunostaining for VE-Cadherin on day 6. Gaps within the within the endothelial monolayer are indicated by arrows.

FIGS. 25a-25c show immunostained VE-Cadherin junctions for channels endothelialized with GFP HUVECs (30M mL⁻¹) after being exposed to different media compositions. FIG. 25a shows endothelialized gel perfused with standard, complete vasculife media containing VEGF for six days at a flow rate of 5 μL min⁻¹. FIG. 25b shows endothelialized gel perfused with complete vasculife media containing VEGF for three days, then switched to a non-VEGF containing vasculife media for the next three days; perfused for a total of six days at 5 μL min⁻¹. FIG. 25c shows endothelialized gel perfused with complete vasculife media containing VEGF for three days, then switched to a media comprised of complete vasculife media (containing VEGF) with Angiopoeitin-1 (Ang-1) added (15 ng mL⁻¹). This gel was sustained for a total of twelve days while being perfused at 5 μL min⁻¹.

FIGS. 26a-26b shows effects of Ang-1 addition into perfusion media on GFP HBMEC and VE-Cadherin morphology after 6 days in perfusion culture. GFP HBMECs was seeded in the vascular channel at 30M mL⁻¹and perfused at 5 μL min⁻¹for duration of the study. FIG. 26a shows GFP HBMEC serpentine perfused with complete vasculife media containing VEGF for three days, then switched to a media comprised of complete vasculife media (containing VEGF) with Angiopoeitin-1 (Ang-1) added (15 ng mL⁻¹) and perfused for an additional three days, for a total of six days perfusion. FIG. 26b shows magnified ROI of center channel (dashed box), to highlight cell-cell junction morphology displaying VE-Cadherin, GFP HBMEC, and Hoechst/nuclei. Overlay and individual channels of GFP HBMEC and VE-Cadherin shown.

FIGS. 27a-27d show sequential co-culture of GFP HBMECs and RFP HBMPs within serpentine channel. RFP HBMPs was seeded in acellular printed serpentine channel (30M mL⁻¹) on day 0 and GFP HBMECs was seeded into channel (30M mL⁻¹) on day 3. The channel was perfused with complete vasculife media at 5 μL min⁻¹for duration of the study. FIG. 27a shows 4× stitch of entire serpentine channel to visualize presence of cellular layer along entire architecture. FIG. 27b shows the center channel ROI (dashed box) with overlay of GFP HBMEC and RFP HBMP). FIG. 27c shows the center channel ROI with only GFP HBMEC to visualize endothelial cell morphology and coverage. FIG. 27d shows center channel ROI with only RFP HBMP to visualize pericyte morphology and coverage.

FIGS. 28a-28c show pericytes covering the edges of the channel by wrapping around the endothelial layer in the sequential co-culture of GFP HBMECs and RFP HBMPs within the serpentine channel. RFP HBMPs were seeded in acellular printed serpentine channel (30M mL⁻¹) on day 0 and GFP HBMECs were seeded into channel (30M mL⁻¹) on day 3. The channel was perfused with complete vasculife media at 5 μL min⁻¹for the duration of the study. FIG. 28a shows the focal plane in the z dimension of the channel is the bottom surface of the channel.

FIG. 28b shows the focal planes in the z dimension along the edges of the channel (two z positions shown for edges). FIG. 28c shows the focal plane in the z dimension of the channel is the top surface of the channel.

FIG. 29 shows straight channel model architecture. A 3D model of the hydrogel construct showing the dimensions and geometry of the perfusable gels used for the BBB model.

FIGS. 30a-30d show longitudinal progression of tri-culture within a 3D printed perfused straight channel design. RFP HBMPs were seeded (30M mL⁻¹) in HBA-printed (6M mL⁻¹) straight channel on day 0 and GFP HBMECs were seeded into channel (30M mL⁻¹) on day 3. The channel was perfused with complete vasculife media at 5 μL min⁻¹for duration of the study. FIG. 30a shows a 4× stitch of entire straight channel to visualize presence of cellular layer along entire architecture. FIG. 30b shows a center channel ROI (dashed box) with overlay of GFP HBMEC and RFP HBMP). FIG. 30c shows a center channel ROI with only GFP HBMEC to visualize endothelial cell morphology and coverage. FIG. 30d shows a center channel ROI with only RFP HBMP to visualize pericyte morphology and coverage.

FIGS. 31a-31d show a cross section of a perfused straight channel hydrogel tri-culture. FIG. 31a shows a cross section of the entire gel. Human brain astrocytes (HBAs) are printed uniformly throughout the gel matrix, while GFP HBMECs and human brain microvascular pericytes (RFP HBMPs) line the perfused channel. The hydrogel was printed with HBAs (6M mL⁻¹) and sequentially seeded with pericytes (30M mL⁻¹) on day 1 and endothelial cells (30M mL⁻¹) on day 3. The image shown is a cross section from day 6 culture overall (day 3 endothelial cell culture). FIG. 30b shows a magnified region from (a) of the bulk printed gel, demonstrating the rounded morphology of astrocytes that is seen in the bulk matrix of the gel. FIG. 30c shows a magnified region from (a) of the channel demonstrating more elongation of the astrocytes near the channel. FIG. 30c shows a magnified region from (c) were taken at different z positions and highlight unlabeled astrocytes elongating near the channel (arrows). FIG. 30d shows a magnified channel cross section.

FIGS. 32a-32d shows pilot work in functional assessment of tri-culture hydrogels assessing permeability using 10 kDa Cascade Blue Dextran. Hydrogel was printed with HBAs (6M mL⁻¹) and sequentially seeded with pericytes (30M mL⁻¹) on day 1 and endothelial cells (30M mL⁻¹) on day 3. Work shown is from day 6 culture overall (day 3 endothelial cell culture) after perfusion with complete Vasculife media for 6 days at 5 μL min⁻¹. FIG. 32a shows the ROI of the center of the straight channel tri-culture before perfusion with 10 kDa Cascade Blue Dextran (1 mg mL⁻¹). FIG. 32b shows the ROI of the center of the straight channel tri-culture immediately at the beginning of permeability assessment with Cascade Blue Dextran (t=0 minutes). FIG. 32c shows the ROI of the center of the straight channel tri-culture at the end of permeability assessment with Cascade Blue Dextran (t=15 minutes). FIG. 32(d) shows s screenshot of graphic user interface (GUI) modified from ultrasound-permeability work to quantify the vascular permeability in a reproducible, high-throughput manner.

FIGS. 33a-33b show the screening capabilities with this 3D printed tri-culture in vitro BBB model. FIG. 33a show a tri-culture serpentine hydrogel model after being printed with astrocytes at 6M mL⁻¹, seeded with 30M mL⁻¹RFP HBMP on day 0, and seeded again with 30M mL⁻¹ GFP HBMEC. The construct is perfused with Vasculife media at 5 μL min⁻¹ prior to ultrasound-permeability studies on day 3. FIG. 33b shows the screening application with a model therapeutic compound perfused through the assembled BBB vasculature with ultrasound applications.

FIGS. 34a-34c show a perfusable, incubative stage top system. FIG. 34a shows a perfusable, stage top incubation system setup. The system includes (from left to right) a temperature and gas control box, humidifier, syringe pump, stage top incubator, epifluorescent microscope and waste collection. FIG. 34b shows detailed images of stage top incubator and modifications for perfusion culture. Displaying the different components of the stage top incubator, including the lid and base heating elements, the humidity sensor, the inlet/outlet port modifications, and the gas port for humidified air as well as a custom perfusion chamber and perfusion chamber holder 3D printed in PLA. FIG. 34c shows a 3D model of design of perfusion chamber, specifically aperfusion chamber design that enables n=2 for the option of assessing multiple conditions simultaneously, comparing to a control condition or ensuring reproducibility.

FIGS. 35a-35b show serpentine lumenized anchor model architecture. FIG. 35a shows a hydrogel rendering of the lumenized anchor design comprised of a suspended perfusable hydrogel channel supported by hydrogel pillars underneath and an empty chamber in the center of the hydrogel that enables the casting of additional materials or cells. FIG. 35b shows photographs of the printed hydrogel with cancer cells (344 SQ lung adenocarcinoma cells) cast into the chamber (both left and right) and India ink perfused through the vascular channel (right).

### DETAILED DESCRIPTION

In some embodiments, 3D printed perfusable in vitro BBB models are disclosed. These models can be used as scalable screening tools, and may enable tri-culturing of endothelial cells, pericytes, and astrocytes. In some embodiments, the devices are constructed as a half-pipe (i.e., with a groove) to simplify the culturing while maintaining the 3D-oriented cellular interaction. The provided examples demonstrate the expression of key receptor expressions on the tri-culture model. Additionally, as the examples disclose, media compositions have been developed to accommodate the tri-culture model.

### Definitions

So that the disclosure may be more readily understood, certain technical and scientific terms are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise.

The term “about” in quantitative terms refers to plus or minus 10% of the value it modifies (rounded up to the nearest whole number if the value is not sub-dividable).

A “receiving feature” refers to a structure, trait or construct devised to store, bear, collect, or support a material therein or thereon.

A “channel” refers to a receiving feature in the form of an enclosed passage or a substantially enclosed passage. For example, blood vessels have channels for transporting blood. Some of the BBB models disclosed here have channels. A channel may be tubular (e.g., with two openings), and it may have a changing cross-section. The term “tubular” is not to be taken as exact geometric term, but rather a long, hollow body with a predefined closed cross-section.

A “groove” refers to a receiving feature in the form of a depression that allows passage or that receives a material. Some of the BBB models disclosed here have grooves.

### Hydrogel Components and Methods of Making Them

The devices disclosed herein comprise a hydrogel component. In some embodiments, the hydrogel component surrounds a receiving feature (e.g., a channel) formed between a first opening and a second opening of the hydrogel component. In other embodiments, the hydrogel component forms a groove.

In each of these types of embodiments, the hydrogel component may comprise astrocytes. In some embodiments, the astrocytes are human brain astrocytes.

In some embodiments, the hydrogel component additionally comprises poly(ethylene glycol) diacrylate (PEGDA), lithium phenyl-2,4,6-trimethylbenzoylphosphinate (LAP), gelatin methacrylate (GelMA), or a combination thereof (e.g., all three).

The hydrogel component can be made via various methods. In some embodiments, the methods of making the hydrogel component include preparing a pre-hydrogel mixture that comprises poly(ethylene glycol) diacrylate (PEGDA), lithium phenyl-2,4,6-trimethylbenzoylphosphinate (LAP), and gelatin methacrylate (GelMA); suspending astrocytes in the pre-hydrogel mixture to obtain a hydrogel composition; and 3D printing the hydrogel composition using projection stereolithography to obtain the hydrogel component of the device.

In some embodiments, the hydrogel component comprises 2.25 to 4.25 wt % 3.4 kDa PEGDA. In some embodiments, the hydrogel component comprises 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, or 4.25 wt % 3.4 kDa PEGDA. In some embodiments, the hydrogel component comprises about 2.25, about 2.3, about 2.35, about 2.4, about 2.45, about 2.5, about 2.55, about 2.6, about 2.65, about 2.7, about 2.75, about 2.8, about 2.85, about 2.9, about 2.95, about 3, about 3.05, about 3.1, about 3.15, about 3.2, about 3.25, about 3.3, about 3.35, about 3.4, about 3.45, about 3.5, about 3.55, about 3.6, about 3.65, about 3.7, about 3.75, about 3.8, about 3.85, about 3.9, about 3.95, about 4, about 4.05, about 4.1, about 4.15, about 4.2, or about 4.25 wt % 3.4 kDa PEGDA.

In some embodiments, the hydrogel component comprises 7 to 27 mM LAP. In some embodiments, the hydrogel component comprises 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, or 27 millimolar LAP. In some embodiments, the hydrogel component comprises about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, or about 27 millimolar LAP.

In some embodiments, the hydrogel component comprises 5 to 15 wt % GelMA. In some embodiments, the hydrogel component comprises 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, or 15 wt % GelMA. In some embodiments, the hydrogel component comprises about 5, about 5.25, about 5.5, about 5.75, about 6, about 6.25, about 6.5, about 6.75, about 7, about 7.25, about 7.5, about 7.75, about 8, about 8.25, about 8.5, about 8.75, about 9, about 9.25, about 9.5, about 9.75, about 10, about 10.25, about 10.5, about 10.75, about 11, about 11.25, about 11.5, about 11.75, about 12, about 12.25, about 12.5, about 12.75, about 13, about 13.25, about 13.5, about 13.75, about 14, about 14.25, about 14.5, about 14.75, or about 15 wt % GelMA.

In some embodiments, the hydrogel component comprises astrocytes at a concentration of at least 3 million cells per milliliter and at most 9 million cells per milliliter. In some embodiments, the astrocytes are at a concentration of 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, or 9 9 million cells per milliliter. In some embodiments, the astrocytes are at a concentration of about 3, about 3.2, about 3.4, about 3.6, about 3.8, about 4, about 4.2, about 4.4, about 4.6, about 4.8, about 5, about 5.2, about 5.4, about 5.6, about 5.8, about 6, about 6.2, about 6.4, about 6.6, about 6.8, about 7, about 7.2, about 7.4, about 7.6, about 7.8, about 8, about 8.2, about 8.4, about 8.6, about 8.8, or about 9 9 million cells per milliliter.

In some embodiments of the methods of making the hydrogel components, 3D printing forms a receiving feature, such as a groove on the hydrogel component. In some embodiments of the methods of making the hydrogel components, 3D printing forms a receiving feature, such as a channel inside the hydrogel component.

### Model Devices and Methods of Making Them

In some aspects, the disclosed devices are for use as BBB models. These models may include a hydrogel component, which in some embodiments surrounds a channel formed between a first opening and a second opening of the hydrogel component, or a hydrogel component that forms a groove.

The hydrogel component of the devices of any of the embodiments may include astrocytes. In addition, the devices may also include pericytes disposed in the channel or the groove, and include endothelial cells disposed in the channel or the groove.

In some embodiments, the astrocytes are human brain astrocytes. In some embodiments, the pericytes are human brain microvascular pericytes. In some embodiments, the endothelial cells are human brain microvascular endothelial cells.

In some embodiments, the pericytes adhere to the hydrogel component. In some embodiments, the pericytes adhere to the hydrogel component via one or more astrocytes. In some embodiments, the endothelial cells adhere to the hydrogel component. In some embodiments, the endothelial cells adhere to the hydrogel component via one or more astrocytes. In some embodiments, the pericytes are in direct contact with the endothelial cells. In some embodiments, said direct contact is on a side of the endothelial cells that faces the hydrogel component.

In some embodiments, the channel has a diameter of at least 200 micrometers and at most 2 millimeters. In some embodiments, the channel has a diameter of 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 micrometers. In some embodiments, the channel has a diameter of about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, or about 2000 micrometers.

In some aspects, the devices further comprise a housing chamber enclosing at least a portion of the hydrogel component, wherein the housing chamber has a first port aligned with the first opening and has a second port aligned with the second opening. In some embodiments, the housing chamber is made from a plastic.

In some embodiments of any of the disclosed aspects, the endothelial cells express one or more BBB transporters. In some embodiments, the one or more BBB transporters comprise p-glycoprotein 1 efflux pump, BCRP efflux pump, transferrin receptor, or low-density lipoprotein receptor-related protein 1. In some embodiments, the one or more BBB transporters comprise p-glycoprotein 1 efflux pump, BCRP efflux pump, transferrin receptor, low-density lipoprotein receptor-related protein 1, or a combination thereof. In some embodiments, the one or more BBB transporters comprise p-glycoprotein 1 efflux pump, BCRP efflux pump, transferrin receptor, and low-density lipoprotein receptor-related protein 1.

In some aspects, methods of making the disclosed devices comprise making a hydrogel component using any one of the methods for making a hydrogel surrounding a channel disclosed herein; introducing pericytes at a concentration of 20 to 40 million cells per milliliter into the channel; and introducing endothelial cells at a concentration of 20 to 40 million cells per milliliter into the channel. In some embodiments, the endothelial cells are introduced into the channel after the pericytes.

In some aspects, methods of making the disclosed devices comprise making a hydrogel component using any one of the methods for making a hydrogel forming a groove disclosed herein; introducing pericytes at a concentration of 0.2 to 0.6 million cells per milliliter into the groove; and introducing endothelial cells at a concentration of 0.2 to 0.6 million cells per milliliter into the groove. In some embodiments, the endothelial cells are introduced into the groove after the pericytes.

### Methods of Using the Devices

In some aspects, methods of selecting a neurotherapeutic compound comprise introducing a plurality of neurotherapeutic compounds, either separately or collectively, to the receiving feature (e.g., a channel or a groove) of one or more of the disclosed devices; determining the relative permeabilities of the plurality of neurotherapeutic compounds into the hydrogel component; and selecting at least one neurotherapeutic compound based on its determined relative permeability.

In some embodiments, a single neurotherapeutic compound is introduced into a channel or groove of one device, and another neurotherapeutic compound is introduced into a channel or groove of another device. In some embodiments, a single neurotherapeutic compound is introduced into a channel or groove of a device, and another neurotherapeutic compound is introduced at a different time into a channel or groove of the same device. As long as the relative permeabilities of different compounds can be determined, one can use one or more devices and can perform the introduction step at the same or different times.

In some embodiments, the methods further comprise applying ultrasound to said one or more devices after introducing the plurality of neurotherapeutic compounds, either separately or collectively, to said channel or groove.

EXAMPLES

The following examples are provided to promote a further understanding of the present disclosure.

### Example 1: Development of a 3D Printed Perfusable In Vitro BBB Model for Use as a Scalable Screening Tool

The following examples show the development of a 3D printable hydrogel BBB model that mimics the cellular composition and arrangement of the BBB with human brain endothelial cells lining the surface, pericytes in direct contact with the endothelial cells on the abluminal side of the endothelium, and astrocytes in the surrounding printed bulk matrix. It will be understood that any of the examples provided are contemplated including intermediary or suboptimal models. In some examples, a simple, static printed hemi-cylinder model is used to determine design parameters such as media selection, co-culture ratios, and cell incorporation timing in a resource-conservative and high-throughput manner. Presence of cellular adhesion junction, VE-Cadherin, efflux transporters, P-gp and Breast cancer resistance protein, and receptor-mediated transporters, TfR and low-density LRP-1 were confirmed via immunostaining demonstrating the ability of this model for screening in therapeutic strategies that rely on these transport systems. Design parameters determined in the hemi-cylinder model may be translated to a more complex, perfusable vessel model to demonstrate its utility for determining barrier function and assessing permeability to model therapeutic compounds. This 3D-printed BBB model uses projection stereolithography to fabricate perfusable a BBB model, enabling the patterning of complex vessel geometries and precise arrangement of cell populations. This model demonstrates potential as a new platform to investigate the delivery of neurotherapeutic compounds and drug delivery strategies through the BBB, providing a useful in vitro screening tool in central nervous system drug discovery and development.

### 2D Immunostaining of VE-Cadherin

In a first example, Green Fluorescent Protein Human Brain Microvascular Endothelial Cells (GFP HBMECs) were seeded in a 24 well plate at ˜200 k cells cm⁻² in 1 mL per well of varying media types. The medias used for these studies were Vasculife Complete Media (Lifeline Cell Technology), Endothelial Growth Media (Angio-Proteomic), and Pericyte Growth Media (Angio-Proteomic). After culturing for 3 days with daily media changes, the cell monolayers were fixed with 4 wt % paraformaldehyde (PFA) for 10 minutes, followed by 20 minutes of permeabilization with 0.1% Triton X-100 in phosphate-buffered saline (PBS). Then cells were blocked with 1 wt % bovine serum albumin (BSA) for 1 hour. Primary antibody for VE-Cadherin (VE-Cadherin, D87F2 Rabbit mAb, Cell Signaling Technology) was prepared 1:400 in 1 wt % BSA solution and 100 μL was added to each respective well to incubate overnight at 4° C. Following overnight incubation, 3×30 min washes with PBS were done before incubating with the secondary antibody (Anti-rabbit IgG (H+L) #4413, Alexa Fluor® 555 Conjugate, Cell Signaling Technology) prepared at 1:500 for 2 hours. Finally, a nuclear counter-stain was applied using 2.5 μg mL⁻¹ Hoechst for 15 minutes. Finally, all wells were washed 3×30 minutes with PBS before imaging. Images were acquired on Nikon Ti epifluorescent microscope at 20× magnification.

### Printing Astrocyte-Containing 2.5D Hydrogel Half Pipes

In some examples, the polymers and initiators for the pre-hydrogel solutions, including poly(ethylene glycol) diacrylate (PEGDA; 3.4 kDa), lithium phenyl-2,4,6-trimethylbenzoylphosphinate (LAP), gelatin methacrylate (GelMA) were synthesized. For fabrication of cellular monolithic gels, pre-hydrogel mixtures were prepared containing 10 wt % GelMA, 3.25 wt % 3.4 kDa PEGDA, 17 mM LAP, 2.255 mM tartrazine photo absorber, and 10% glycerol in sterile 1× PBS. Immediately before printing, human brain astrocytes (HBAs) (passages 5-7) were resuspended in the pre-hydrogel mixture at a concentration of 6M cells mL⁻¹.

This 2.5D model of a hydrogel half pipe was created in Blender and designed with both flat and curved hemi-spherical surfaces surrounded by 1 mm tall hydrogel walls that hold a volume of −50 μL of solution. After printing is completed, the 2.5D half pipe is removed from the glass slide of the build platform with a sterile razor and equilibrated in Vasculife media supplemented with 1% penicillin/streptomycin. Multiple Vasculife media washes are performed to remove any unreacted moietics.

### Pericyte Seeding on Astrocyte-Containing Half Pipes

In some examples, the HBA-containing hydrogel half-pipes were soaked in Vasculife media supplemented with 1% penicillin/streptomycin overnight in individual wells of a 24-well plate. RFP expressing human brain microvascular pericytes (RFP HBMPs) (passages 5-7) were re-suspended from freeze and pipetted onto the hydrogel surface at 400 k cells cm⁻². To allow HBMP adhesion, the half pipes were seeded for 4 hours at 37° C. After the seeding period, gels were covered with 1 mL of complete Vasculife media supplemented with 1% penicillin/streptomycin. RFP HBMPs were cultured on the HBA-containing hydrogel half pipes for three days before the addition of endothelial cells. Media changes were conducted daily using Vasculife media supplemented with 1% penicillin/streptomycin. Images were acquired daily on Nikon Ti epifluorescent microscope at 4×, 10× or 20× magnification.

### Endothelial Cell Seeding on Pericyte and Astrocyte-Containing Half Pipes

In some examples, after three days of pericyte culture, GFP expressing human brain microvascular endothelial cells (GFP HBMECs) (passages 5-7) were seeded on top of the HBA and RFP HBMP-containing hydrogel half pipe. GFP HBMECs were re-suspended from freeze and pipetted onto the pericyte-filled hydrogel surface at 400 K cm⁻², creating a 1:1 ratio of pericytes to endothelial cells on top of the astrocyte-containing printed half pipe. To enable HBMEC adhesion, the half pipes were seeded for 4 hours at 37° C. After the seeding period, gels were covered with 1 mL of complete Vasculife media supplemented with 1% penicillin/streptomycin. After the addition of the GFP HBMECs, the tri-culture hydrogel half pipes were carried out for six days (for a total culture time of ten days for the HBAs printed in the bulk, nine days for the RFP HBMPs seeded on top the hydrogel, and six days for the GFP HBMECs seeded on top). Media changes were conducted daily using Vasculife media supplemented with 1% penicillin/streptomycin. Images were acquired daily on Nikon Ti epifluorescent microscope at 4×, 10× or 20× magnification.

### Immunostaining of VE-Cadherin and BBB Transporters on Tri-Culture Hydrogel Half Pipes

In some examples, tri-culture hydrogel half-pipes were cultured with complete Vasculife media supplemented with 1% penicillin/streptomycin for 6 days after addition of the final cell type, GFP HBMECs. For the following staining steps, per hydrogel 50 μL of each solution was pipetted onto the surface sequentially being careful not to puncture the hydrogel with the pipette tip. First, the cell monolayers were fixed with 4 wt % paraformaldehyde (PFA) and allowed to soak for 10 minutes at room temperature, followed by 20 minutes of permeabilization with 0.1% Triton X-100 in PBS. Then cells were blocked with 1 wt % bovine serum albumin (BSA) for 1 hour at room temperature. Primary antibody for VE-cadherin (VE-Cadherin, D87F2 Rabbit mAb, Cell Signaling Technology) was prepared at 1:400 in 1 wt % BSA solution and 50 μL pipetted onto each gel to incubate overnight at 4° C. Transporters were stained for using primary antibodies for p-gp 1 (Rabbit polyclonal to P glycoprotein, Abcam, ab235954), BCRP (Rabbit monoclonal to BCRP/ABCG2, Abcam, ab229193), TIR (Rabbit monoclonal to transferrin receptor, Abcam, ab214039), and low density lipoprotein receptor-related protein 1 (LRP-1) (LRP-1, Rabbit mAb #26387, Cell Signaling Technology). Primary antibodies were prepared at 1:50 in 1 wt % BSA solution and 50 μL pipetted onto each gel to incubate overnight at 4° C. Following overnight incubation, 3×30 min washes with PBS were done before incubating with the secondary antibody (Anti-rabbit IgG (H+L) #4414, Alexa Fluor® 647 Conjugate, Cell Signaling Technology) prepared at a 1:500 dilution for 2 hours. A nuclear counter-stain was applied using 2.5 μg/mL Hoechst for 15 minutes. Finally, all gels were washed 3×30 minutes with PBS before imaging. Images were acquired on Nikon A1-Rsi Confocal at 10× magnification.

### Printing of Astrocyte-Containing 3D Straight Channel Hydrogels

In some examples, for fabrication of cellular 3D gels, the same pre-hydrogel mixture described above was used (10 wt % GelMA, 3.25 wt % 3.4 kDa PEGDA, 17 mM LAP, 2.255 mM tartrazine photo absorber, and 10% glycerol in sterile 1× PBS). Identical to the 2.5D studies, before printing, human brain astrocytes (HBAs) (passages 5-7) were resuspended in the pre-hydrogel mixture at a concentration of 6M cells mL⁻¹. The HBA-containing 3D straight channel hydrogels were soaked in Vasculife media supplemented with 1% penicillin/streptomycin overnight in individual wells of a 6-well plate. Multiple Vasculife media washes are performed to remove any unreacted moietics.

Seeding Pericytes within Astrocyte-Containing 3D Straight Channel Hydrogels

In some examples, the 3D printed hydrogels containing 6M mL⁻¹ HBAs printed into the bulk and a 700 μm diameter straight or serpentine channel were transferred into sterilized 3D printed plastic housing chambers with aligned ports for fluidic tips. Fluidic tips were primed with media and gently inserted into the gel chamber and then into the inlet and outlet portions of the hydrogel. Proper fluidic connection and cellular adhesion were ensured by injecting complete Vasculife media and visibly verifying the absence of leakage into the gel chamber housing. RFP HBMPs were re-suspended from freeze to a density of 30 M cells mL⁻¹ for seeding and slowly injected into the channel via the catheter. To encourage uniform cell adhesion, gels were seeded for 4 hours at 37° C. and rotated 90 degrees every 15 minutes. After the seeding period, gels were perfused with complete Vasculife media supplemented with 1% penicillin/streptomycin at a flow rate of 5 μL min⁻¹. Pericytes were cultured under flow on the within the channel of the HBA-containing printed gel for one to three days (see timeline studies in the results) prior to endothelial cell inclusion.

Seeding Endothelial Cells within Pericyte and Astrocyte-Containing 3D Straight Channel Hydrogels

In some examples, after one to three days of pericyte culture, GFP expressing human brain microvascular endothelial cells (GFP HBMECs) (passages 5-7) were seeded within the channel of the HBA and RFP HBMP-containing 3D straight channel hydrogel. GFP HBMECs were seeded at 30 M cells mL⁻¹, matching the 1:1 ratio of endothelial cells to pericytes that was optimized in the 2.5D half pipe studies. To encourage HBMEC adhesion, gels were seeded for 4 hours at 37° C. and rotated 90 degrees every 15 minutes. After the seeding period, gels were perfused with complete Vasculife media supplemented with 1% penicillin/streptomycin at a flow rate of 5 μL min⁻¹. After the addition of the GFP HBMECs, the tri-culture hydrogel half pipes were carried out for three to six days (for a total culture time of ten days for the HBAs printed in the bulk, nine days for the RFP HBMPs seeded on top the hydrogel, and six days for the GFP HBMECs seeded on top), mimicking the timeline used in the 2.5D studies. Images were acquired daily on Nikon Ti epifluorescent microscope at 4×, 10× or 20× magnification.

### Demonstration of Functional and Therapeutic Screening Studies

In some examples, pilot studies for functional assessment were conducted on tri-culture gels after 6 days of perfusion culture with Vasculife media at a flow rate of 5 μL min⁻¹. On day 6, the tri-culture hydrogel was transferred to the scope and permeability studies were conducted by perfusing 10 kDa Cascade Blue dextran (1 mg mL⁻¹) at 100 μL min⁻¹ for 15 minutes. For the ultrasound-permeability studies, the perfusate used was IgG-AF555 (15 μg mL⁻¹) with microbubbles incorporated in a 1:20 dilution in PBS (FUJIFILM VisualSonics, Item number: VS-11913). Methods for permeability calculation and ultrasound application were replicated as described in sections 3.4.4 and 3.4.5.

### Vasculife Media Produced Superior Endothelial Cell-Cell Junctions on Half Pipes

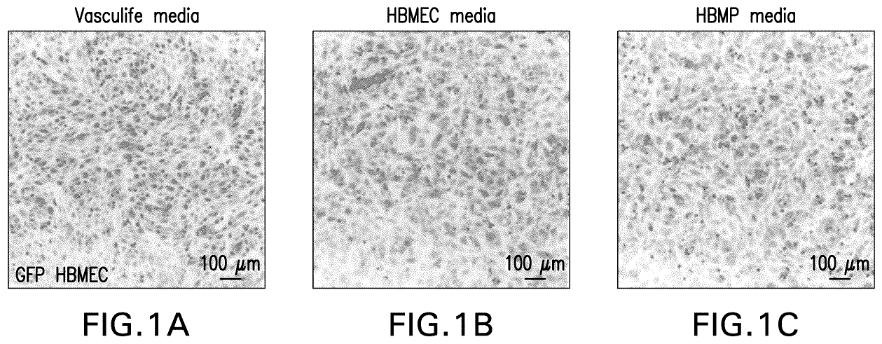

In some examples, GFP HBMECs were plated at 200 K cm⁻² in a 24-well plate and grown for three days prior to assessing morphology between the three media types, which can be seen in FIG. 1. While it is less apparent looking only at the GFP signal, localized to the cell cytoplasm, to discuss morphology, few minor differences can be seen between the endothelial cells grown in different media conditions. The endothelial cells grown in Vasculife media (FIG. 1a) appear smaller and more tightly packed than those in either the HBMEC or HBMP media (FIGS. 1b & 1c, respectively). It appears that the HBMEC and HBMP media produce similar morphologies of endothelial cells.

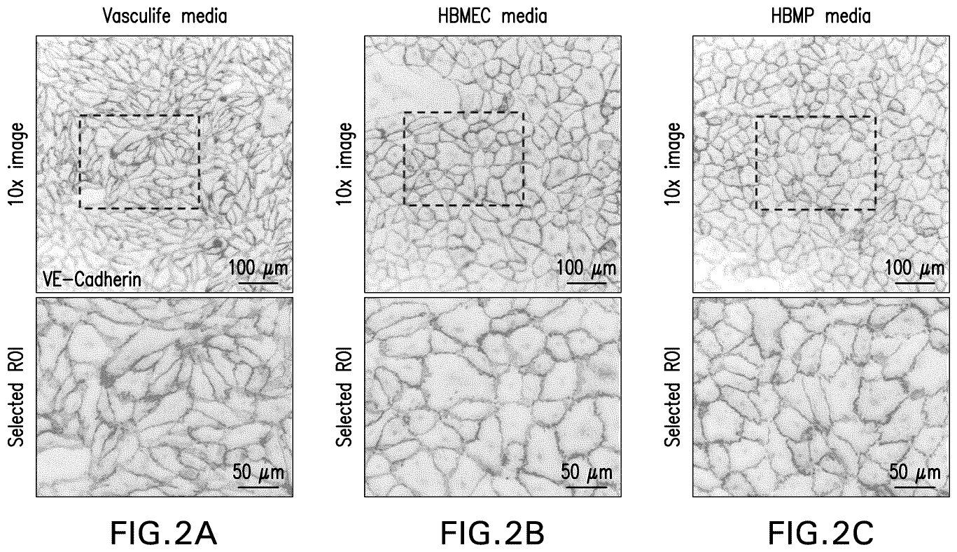

While looking at the morphology of the cell types grown across three cells using their endogenous fluorescence signal provides insight to how the cells change over time, VE-cadherin immunostaining is more informative when assessing the maturity of cell-cell junctions and whether the endothelial cells are forming a confluent endothelium. VE-cadherin immunostaining for the three media conditions after fixing the sample above on day 3 is shown in FIG. 2. The differences between the media types are a bit more apparent when looking at the localization and organization of VE-cadherin within these cell monolayers. The same conclusion can be made that in the Vasculife media condition (FIG. 2a) the endothelial cells appear more elongated and more tightly packed than those in the HBMEC or HBMP media conditions (FIGS. 2b & 2c, respectively). Additionally, it appears that there may be more intracellular VE-Cadherin in the HBMEC and HBMP media conditions, indicative of a less mature monolayer where the VE-Cadherin has been produced in the ribosomes within the cytoplasm but has not yet been transported to and localized at the cell peripheries.

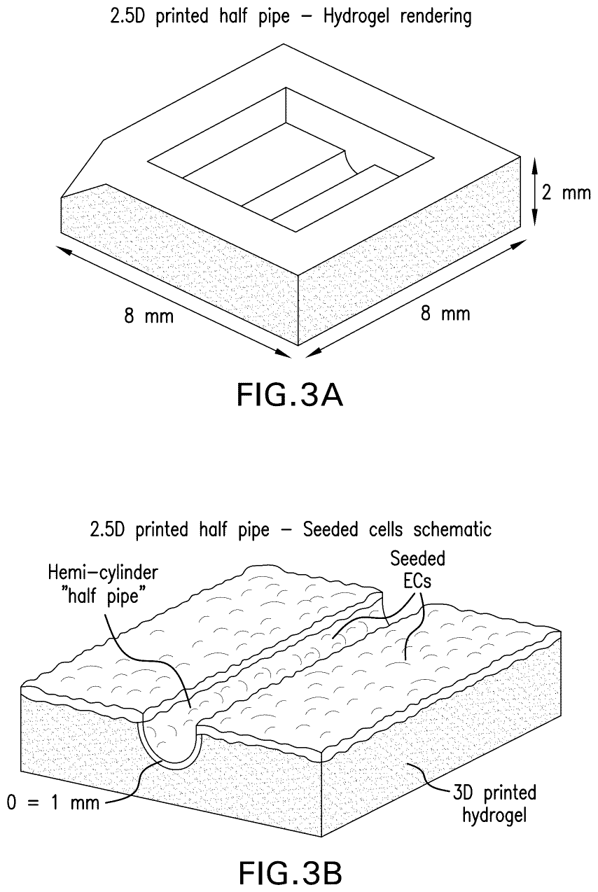

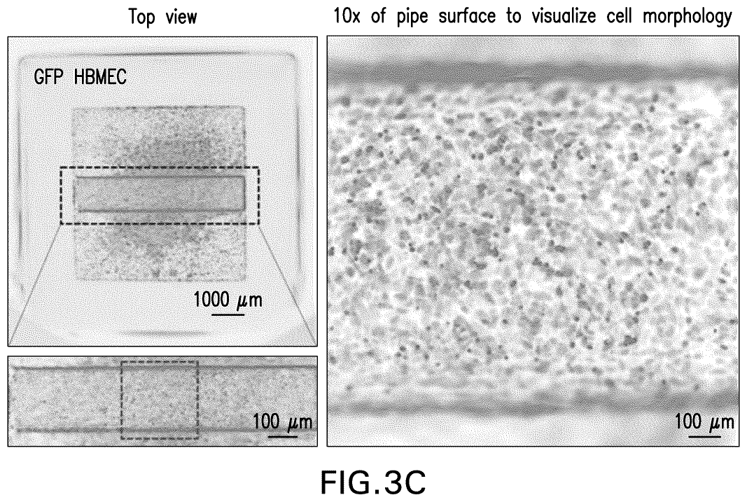

To determine tri-culture parameters, a model printed with the hydrogel formulation for the 3D channels will provide a more accurate representation of cell behavior prior to moving to 3D studies. To do this, a hydrogel hemi-cylinder, or “half pipe” was utilized as a 2.5D model to study cell behavior and attachment to the printed hydrogels. This model architecture includes a curved region (pipe surface) and a flat region (surface) to evaluate adhesion and morphology of cells on both surface topographies. The rendering of this hydrogel model and dimensions can be seen in FIG. 3a. The diameter of the half pipe is roughly one millimeter across and the depth between the surface and the bottom of the half pipe is roughly 500 micrometers. The construct also contains walls on all four sides that extend above the surface that aid in seeding cells on this structure. When seeding cells, 50 μL of cell suspension is placed directly into the half pipe well area, and the walls help to hold the entire volume of cell suspension during the four-hour seeding period. The rendering showing where the cell seeding occurs in the well region between the extended walls is seen in FIG. 3b. When imaging these constructs, the top view is often shown (FIG. 3c), where a region of the curved pipe is often shown for visualizing cell morphology studies (FIG. 3c, right).

These 2.5D printed half pipes may be printed with the same composition and parameters as the 3D channels, may use substantially fewer cells to perform screening studies, may allow for the assessment of how cells attach, spread, and grow on the printed material used, and finally, may allow higher throughput than 3D channel screenings. However, disadvantageous of this 2.5D printed half pipe include static conditions not representative of a 3D perfusion model, and the nutrient and oxygen availability are much higher than the 3D perfusion model, as there is a higher volume of media fully submerging the half pipe, compared to the volume of media perfused through the 3D channel. This model is particularly advantageous for the development of a tri-culture model, where there are several biological questions to be answered. These include determining the optimal media type for the cells on printed gels, selecting a representative ratio of endothelial cells and pericytes, determining a timeline for culture and how each cell type can be incorporated into the model, and finally, performing pilot immunostaining studies examining endothelial cell-cell junctions as well as BBB specific transporters.

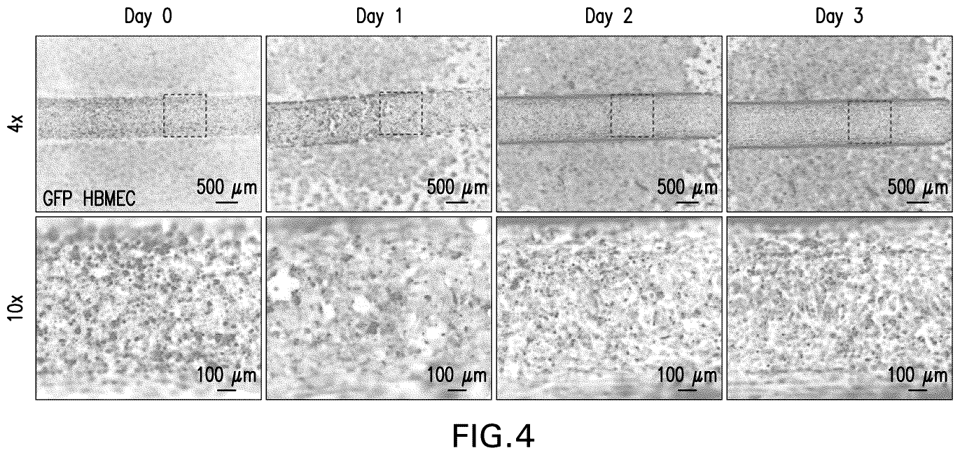

Prior to beginning co-and tri-culture studies with endothelial cells and pericytes, and endothelial cells, pericytes, and astrocytes, respectively, studies were first conducted only with GFP HBMECs to examine their behavior on this 2.5D printed half pipe. FIG. 4 shows the progression of morphology and coverage for endothelial cells on the half pipe through three days of culture after being seeded on day 0 at 400 K cm⁻². When performing the studies with endothelial cells on half pipes, cells were seeded at 400 K cm⁻², which was calculated to be equivalent to the seeding density of 30 M cells mL⁻¹ used in the previous 3D studies. Over the three days of culture, it appears that the endothelial cells are attaching printed gel formulation (seen between days 0 and 1) and are filling in gaps that are present (seen between day 1 to day 3) to form a filled out half pipe that has a confluent endothelium within the pipe surface. Less emphasis was placed on examining the flat morphology of the top surface of these half pipes, as the curved morphology will be more applicable for translating to 3D studies.

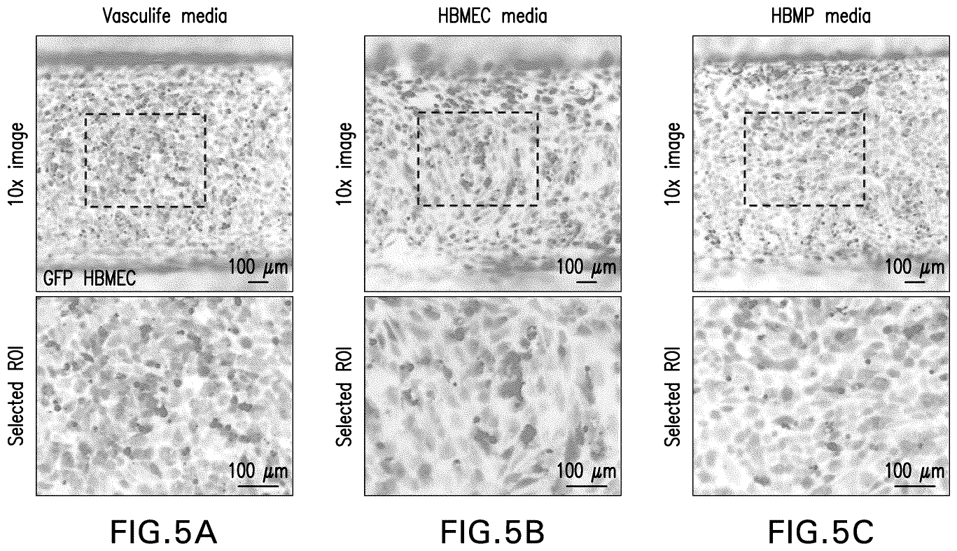

While studies examining endothelial morphology to various medias were conducted in 2D well plates, it was also beneficial to conduct these studies in 2.5D to determine if the endothelial cells displayed any different behaviors to various medias when being cultured on the printed gel formulation. In doing this, the same three media types were examined: complete Vasculife media supplemented with 1% penicillin/streptomycin from Lifeline Cell Technologies, human brain microvascular endothelial cell media from Angio-Proteomic, and human brain microvascular pericyte media from Angio-Proteomic. Similar to the results seen in 2D, it is difficult to make definitive conclusions on cell morphology and maturity by only assessing the GFP signal. One minor difference to be noted is that the endothelial cells cultured on 2.5D half pipes in Vasculife media (FIG. 5a) appear to be smaller and less spread than those grown on 2.5D half pipes in HBMEC media (FIG. 5b) or HBMP media (FIG. 5c). VE-Cadherin immunostaining was conducted on these half pipes after three days of growth to further comment on the endothelial layer.

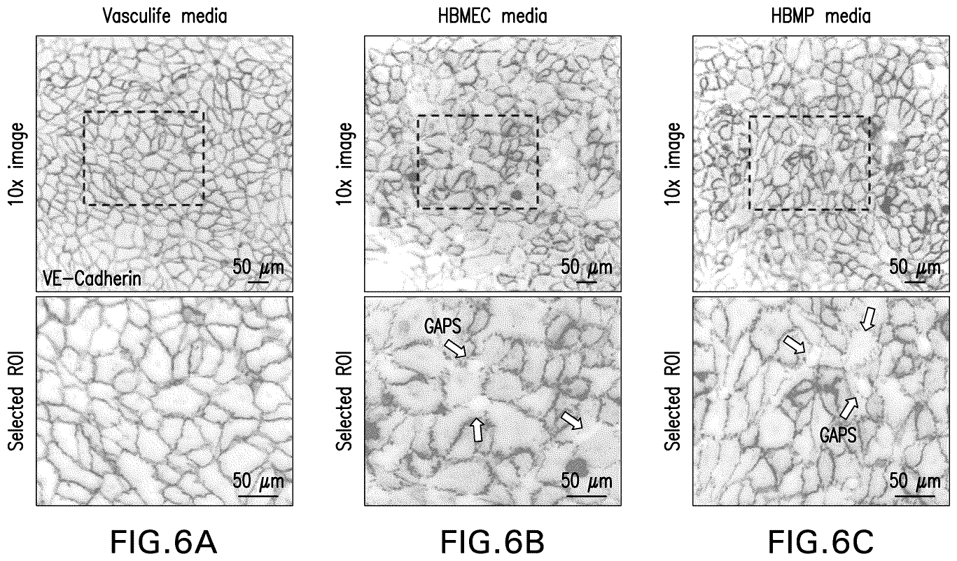

VE-Cadherin immunostaining for these half pipes with endothelial cells after three days of culture according to some examples can be seen in FIG. 6. With the VE-Cadherin immunostaining on the 2.5D half pipes, there is a much clearer result that Vasculife is the superior media to use for this model. The endothelial cells grown in Vasculife media display clearly defined cell-cell junctions that have thick bands surrounding the peripheries of the cells and a low amount of intracellular VE-Cadherin (FIG. 6a). In contrast, the endothelial cells grown on the 2.5D half pipes in HBMEC and HBMP media (FIGS. 6b & 6c) display a higher amount of VE-Cadherin signal intracellularly, more jagged VE-Cadherin morphology, and notable gaps within the endothelial monolayer, indicated by the arrows in the selected ROI row. This underscores the need for screening in 2.5D on the printed half pipes over 2D TC wells, as the endothelial cell behavior differs in 2D when cultured on a plastic substrate compared to softer, more physiologic materials. Moving forward, the co-culture studies conducted with endothelial cells and pericytes, as well as the tri-culture studies utilizing endothelial cells, pericytes, and astrocytes, are performed using complete Vasculife media, as this was the media that the brain microvascular endothelial cells formed the most stable cell-cell junctions in.

The Vasculife Complete Media from Lifeline Cell Technology is a low serum media that is optimized for rapid proliferation of endothelial cells. This media comprises 2% FBS and additional factors including L-glutamine, fibroblast growth factor (FGF), ascorbic acid, hydrocortisone hemisuccinate, insulin-like growth factor, epidermal growth factor, vascular endothelial growth factor, and heparin sulfate. Both the HBMEC media and the HBMP media are from Angio-Proteomic and comprise a basal media that comes already prepared with 10% FBS and a proprietary blend of growth factors. Since the blend of growth factors is not disclosed for the HBMEC and HBMP medias, it is challenging to make any hypotheses relating to the morphology differences seen and if they are a result of growth factors present in the media. One conclusive difference between the medias is the concentration of fetal bovine serum (FBS), where Vasculife has 2% FBS and the HBMEC and HBMP medias contain 10%. FBS is incorporated in cell culture media to promote rapid cell growth. It is possible that the junctional differences seen between media types may be attributed to the differing amounts of FBS in the culture media, as it has been reported in literature that FBS can increase the permeability of tight junctions of epithelial cells.

### Simultaneous Seeding of Endothelial Cells and Pericytes Results in Non-Complete Endothelial Cell Monolayer

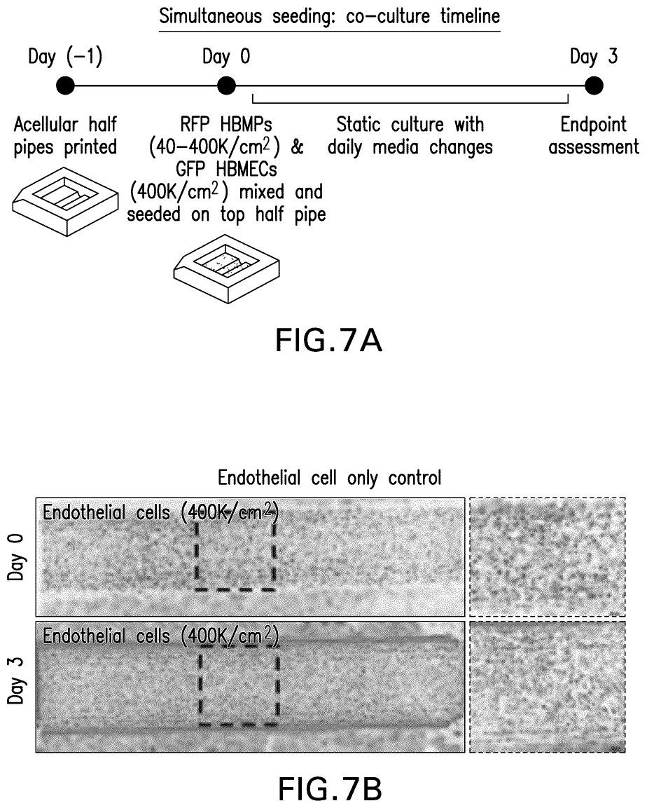

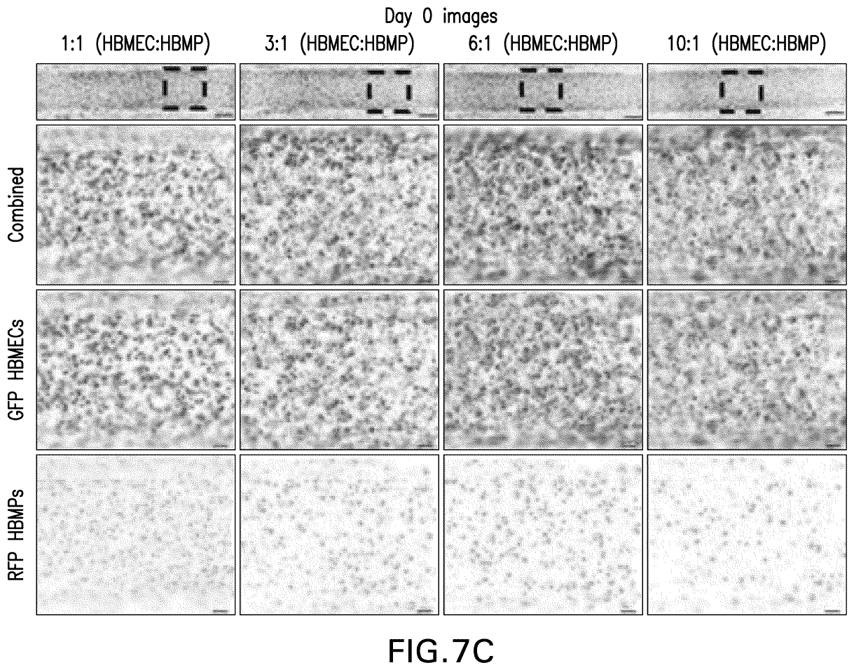

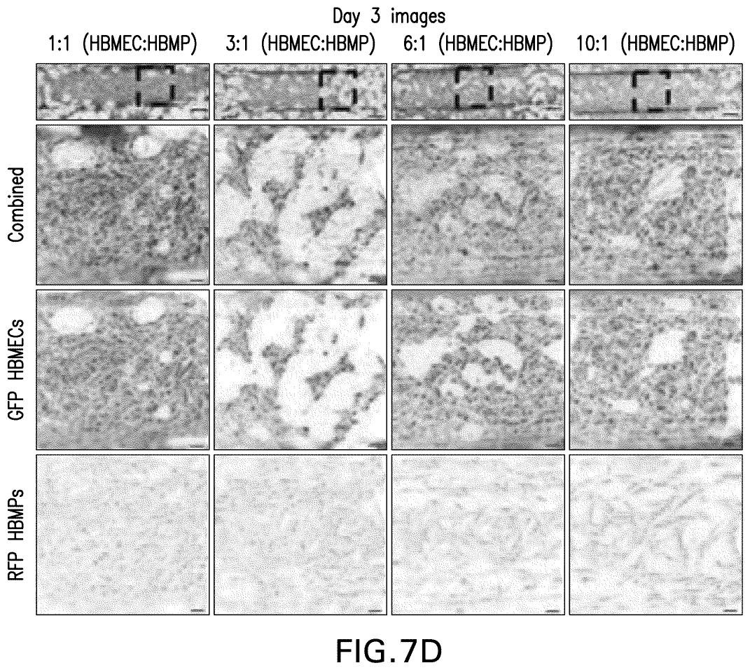

In some examples, Vasculife was decided as the media type to use for co-cultures, and pericytes were incorporated with endothelial cells to determine additional parameters for their co-culture, specifically the timeline for culture and the ratio of endothelial cells to pericytes. This work began with the simultaneous seeding for incorporation of the two cell types. In this workflow, the acellular half pipes were printed and allowed to soak overnight prior to any cell seeding. After allowing to soak overnight, the hydrogel half pipes were seeded with a mixture of RFP HBMPs and GFP HBMECs. To replicate the seeding density used in the 3D channels of past studies (30M cells mL⁻¹), an equivalent surface density of 400 K cells cm⁻² was held constant for the GFP HBMECs in all studies. The seeded half pipes were cultured for three days with daily media changes and morphology was assessed each day. The timeline for this fabrication procedure is depicted in FIG. 7a.

In some examples, the ratio of pericytes was varied between 40 K-400 K cells cm⁻² to mimic ratios of 10:1 to 1:1 endothelial cells to pericytes. In some examples, there are various ratios of endothelial cells to pericytes. In some examples, a 10:1 ratio of endothelial cells to pericytes is contemplated. In some examples, a 5:1 ratio of endothelial cells to pericytes is contemplated. In some examples, the endothelial-to-pericyte ratio varies between 10:1 and 1:1; and this ratio varies throughout the body and is highly organ specific. The central nervous system is generally regarded as having the highest pericyte coverage and is reported to be between a 3:1 and a 1:1 endothelial-to-pericyte ratio. Pericyte density and coverage is reported to positively correlate with endothelial barrier properties, so organs that have a more restrictive endothelium, specifically the brain, generally have a higher pericyte coverage. In some examples, the endothelial-to-pericyte ratios selected for the disclosed studies were 10:1, 6:1, 3:1, and 1:1, or any ration previously noted above. In addition to several co-culture ratios, an endothelial cell only control was also employed (FIG. 7b).

On day 0, the RFP HBMPs and GFP HBMECs were mixed at the determined ratios and placed on the half pipe and allowed to seed for 4 hours. After this seeding period, the half pipes were imaged to assess initial coverage and can be seen in FIG. 7c. In all four ratio conditions as well as the endothelial cell only control, the seeding resulted in a homogenous layer of cells across the entire surface (seen in the first row). One region of the gel was selected and tracked over the course of the three days to assess changes to cell morphology; however, only days 0 and 3 are shown for simplicity. The region was chosen based on a fiduciary marker in the phase image on day 0 to ensure no bias was introduced when assessing the final morphology in the specified region.

The day 3 images are shown in FIG. 7d where all the co-culture conditions displayed an interesting common feature: disruption of the endothelial layer. This can be seen in all conditions across the full pipe in the top row, as well as exemplified in the endothelial cell channel in the third row. In this disruption, rather than the two cell types co-existing in the same location, they take on a mosaic-like arrangement with patches of pericytes and patches of endothelial cells. This arrangement is not ideal, as the endothelium is not able to provide the necessary barrier when there are large gaps in the monolayer. As a result, it was desirable to explore other co-culture routes that may enable the formation of a stable endothelium in the presence of pericytes.

In the body, pericytes surround the abluminal surface of the endothelial tube and form specialized junctions with the endothelial cell. In addition to being in close contact, endothelial cells and pericytes have been described to have three types of contacts: gap junctions, adhesion plaques, and peg-and-socket junctions. Gap junctions enable ionic currents and small molecules to pass between the cytoplasms of the two cell types and adhesion plaques are involved in anchoring the pericyte to the endothelial cell. The last type of contact, peg-and-socket is also described to provide anchorage between the two cells. In peg-and-socket contacts, which occur at discrete points in the basement membrane, cytoplasmic protrusions (pegs) are connected with endothelial cell invaginations (sockets). These peg-and-socket interactions play a role in facilitating endothelial and pericyte adhesion, as well as biochemical crosstalk during vascular homeostasis. The number of endothelial cell and pericyte contacts varies throughout tissues of the body, but up to 1,000 contacts have been described for a single endothelial cell. One hypothesis as to why the gaps in the endothelial monolayer occurred during the simultaneous seeding is that placing the two cell types on at the same time did not enable the formation of the necessary endothelial cell-pericyte contacts. The formation of proper contacts may benefit from an initial attachment and stabilization period for the pericytes before the endothelial cells are added to the hydrogel model so that the proper contacts facilitating endothelial cell-pericyte adhesion are formed. Additional pericyte culture time may enable production and deposition of basement membrane components by the pericytes, also aiding in endothelial adhesion and retention.

### Sequential Seeding of Pericytes and Endothelial Cells Results in Stable Co-Culture

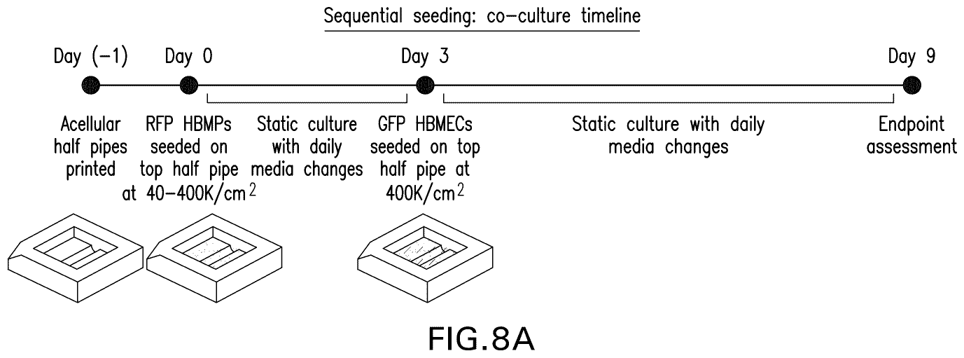

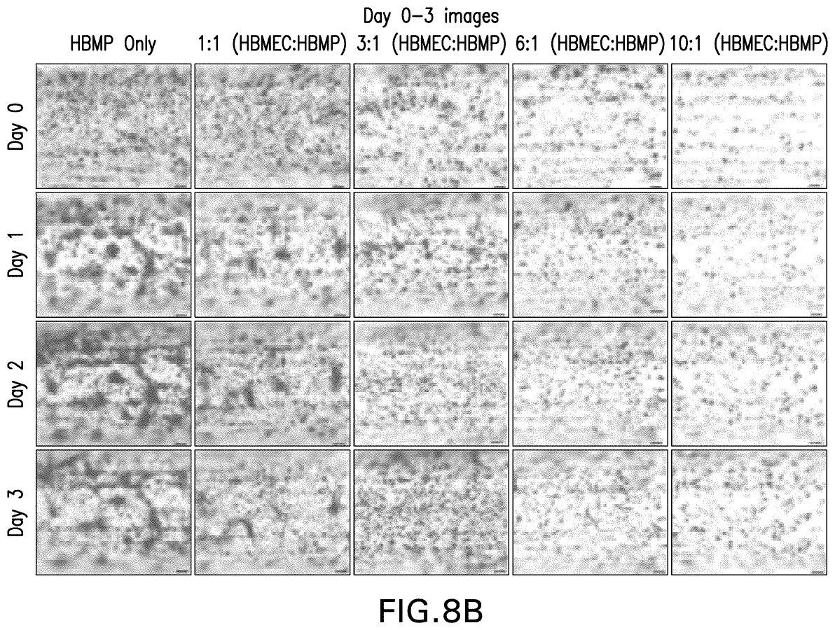

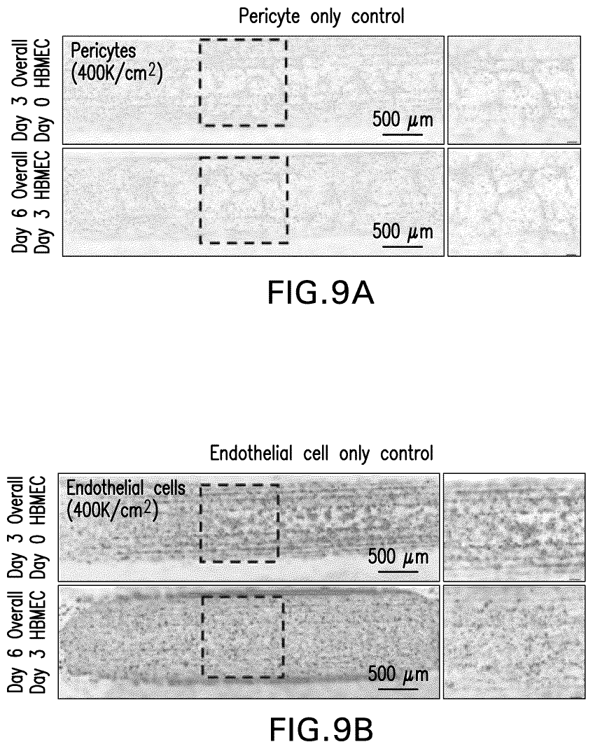

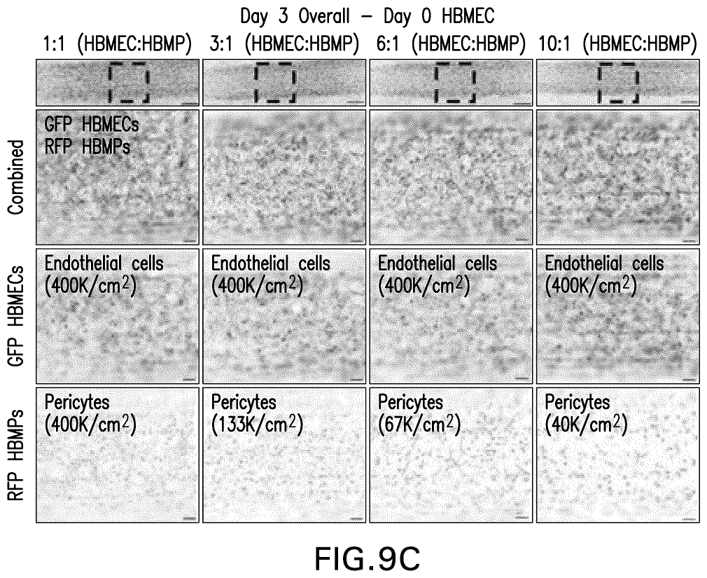

The next parameter that was explored was timing of cell incorporation. It was hypothesized that a stable monolayer of endothelial cells may be achievable if the pericytes have already established coverage on the half pipe. In this sequential approach, the pericytes are still seeded one day after printing, however, they have three days of culture on the pipe before the endothelial cells are added on. This parameter could be further optimized in assessing whether seeding the endothelial cells after one, two, or three days has the same effect and allows for a quicker timeline to the final construct. After the pericytes have been cultured for three days, the endothelial cells are seeded on top, and the construct is cultured for an additional six days. This results in a model that reaches its final form within ten days, from printing to seeding the two cell types, and this is depicted in FIG. 8a. For these studies, the half pipes were imaged every day to assess the daily shifts in morphology, however, only images every three days are shown for simplicity.

For concentrations of pericytes seeded on the half pipe, it can be seen that the morphology of the pericytes does not differ or change substantially over the three days of culture (FIG. 8b). The pericytes appear balled up and do not spread along the surface of the half pipe for the first two days of culture. By day 3, some of the pericytes do begin to spread along the surface of the gel, however, it is a small percentage of the total cells.

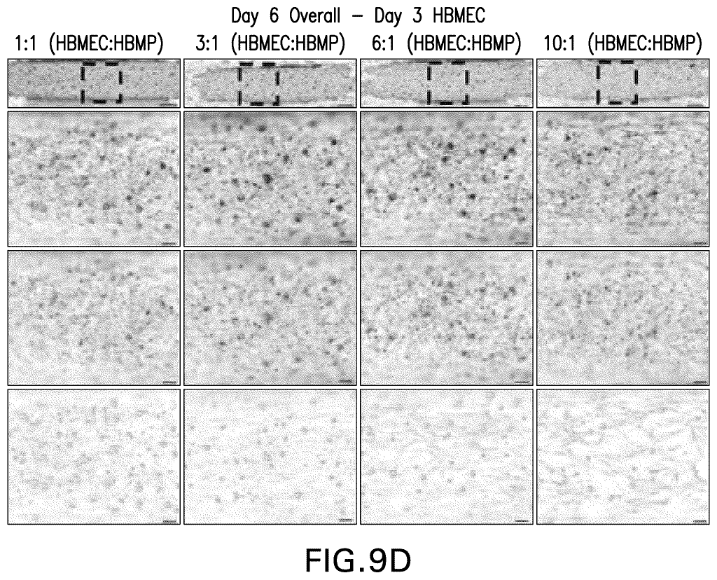

On day 3, endothelial cells were added to the surface of the half pipe at a seeding density of 400 K cells cm⁻², resulting in four different co-culture endothelial-to-pericyte ratios ranging from 1:1 to 10:1. The day 3 images after seeding the GFP HBMECs are seen in FIG. 9a. All ratio conditions and the endothelial cell only control had a high coverage of endothelial cells across the entire surface of the pipe. On day 6 overall (day 3 HBMEC), seen in FIG. 9b, the endothelial cells appear to be spreading along the surface of the gel and have taken on a more rounded, cobblestone morphology. The pericytes have also changed morphology over the three days in co-culture. The pericytes have spread along the surface and have a much more elongated morphology than previously seen. In the pericyte only condition, the pericytes still appear balled up, whereas in each of the co-culture ratio conditions, the pericytes appear to be stretched along the surface of the gel and filling in much of the space. It is hypothesized that this change in morphology is attributed to varying factors secreted by the endothelial cells. One important factor for pericyte function is platelet derived growth factor subunit B (PDGF-B). PDGF-B is secreted by endothelial cells and results in the migration and proliferation of pericytes during vessel maturation. Another secreted factor that plays an important role in endothelial and pericyte function is transforming growth factor beta (TGF-beta). TGF-beta has impacts on both endothelial cells and pericytes, and it is reported that endothelial produced TGF-beta plays a role in pericyte differentiation. However, it should be noted that the production of these factors has not been confirmed but could be done by sampling and freezing the supernatant of the half pipes then performing ELISA for cytokines and secreted factors of interest.

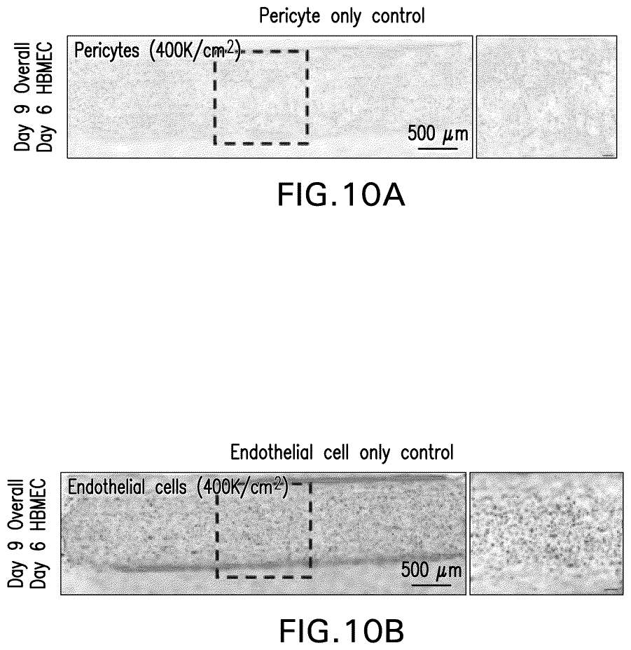

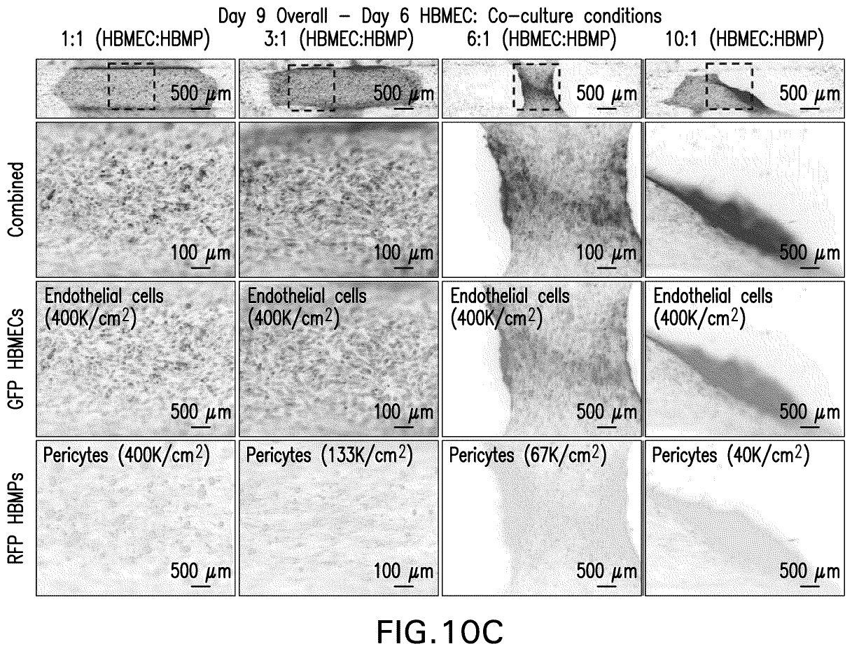

On the final day of culture (day 9 overall, day 6 HBMEC), seen in FIG. 10, an

interesting trend was seen across the varying ratio conditions. In both the pericyte only and endothelial cell only controls, the cells fill the entire length of the pipe. However, in all co-culture ratio conditions, it appears that the co-culture cell layer begins to retract from the edges of the pipe. Interestingly, it appears that both cells are detaching from the pipe surface and beginning to pull inwards towards the center of the pipe. This retraction from the sides of the half pipe is most notable in the conditions where there is a lower ratio of pericytes, specifically the 10:1 and 6:1 conditions. The conditions that had a higher ratio of pericytes (1:1 and 3:1), still displayed some detachment and retraction of the cell layer, however it was less substantial than the lower ratio conditions. The central nervous system has the highest pericyte to endothelial coverage throughout the body, so it is possible for these brain-specific cells that a ratio mimicking in vivo conditions is helpful for proper function. Given this, the 1:1 ratio condition was selected for use in this model and is used in the studies going forward. In the sequential studies, the development of the mosaic-like morphology where gaps form within the individual cell layers like what was seen in the simultaneous studies. It is possible that the culture of pericytes alone for three days enabled the endothelial cells and pericytes to form proper cell contacts once the endothelial cells were added, specifically adhesion plaques and peg-and-socket contacts that are reported to play a role in endothelial cell-pericyte adhesion and tethering.

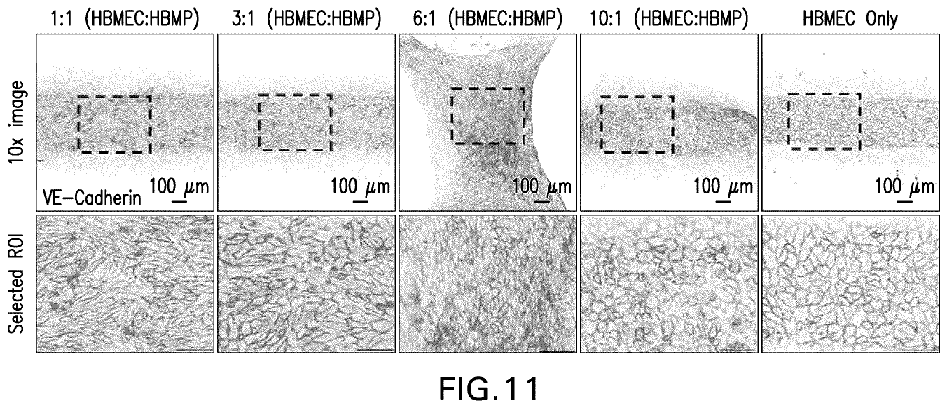

In addition to assessing the morphology of the endothelial cells using their fluorescent cytoplasm, these half pipes were also fixed on day 9 and immunostained for VE-Cadherin to assess the presence and morphology of this endothelial-specific cell-cell junction (FIG. 11). In doing this, VE-Cadherin is seen to be present for all co-culture ratio conditions, as well as the endothelial cell only condition. This shows that the endothelial cells are forming a confluent barrier on our gel as a mono-culture, as well as in co-culture with pericytes. There are slight differences in morphology between the ratio conditions as well as the control. Since the 6:1 ratio condition was retracted into a cell sheet, it will not be included in this discussion. The 1:1 and 3:1 ratio conditions display a VE-Cadherin morphology where it appears that the cells display a more elongated and closely packed phenotype, whereas the cells in the 10:1 and HBMEC only conditions appear to be much more rounded and have more jagged perimeters.

Astrocytes Printed into the Bulk Hydrogel to Mimic In Vivo Location in Surrounding Extracellular Matrix

After the co-culture ratio conditions of endothelial cells and pericytes were determined, the next step was to move towards incorporating the third and final cell type to achieve the BBB tri-culture: astrocytes. Astrocytes are located in the extracellular matrix surrounding blood vessels and have their endfeet extending towards blood vessels. To replicate this arrangement of cells, the integration of astrocytes in this model was accomplished by incorporating astrocytes into the print solution. Human brain astrocytes (HBAs) were printed into the bulk of the hydrogel at a concentration of 6M mL⁻¹. It has been reported that cells and their subcellular structures have the ability to alter the path of incident light, affecting printing resolution via attenuation of ballistic photons or scattering. The scattered light can cause an increase of light dose in non-desired regions, resulting in off-target polymerization and loss of resolution.

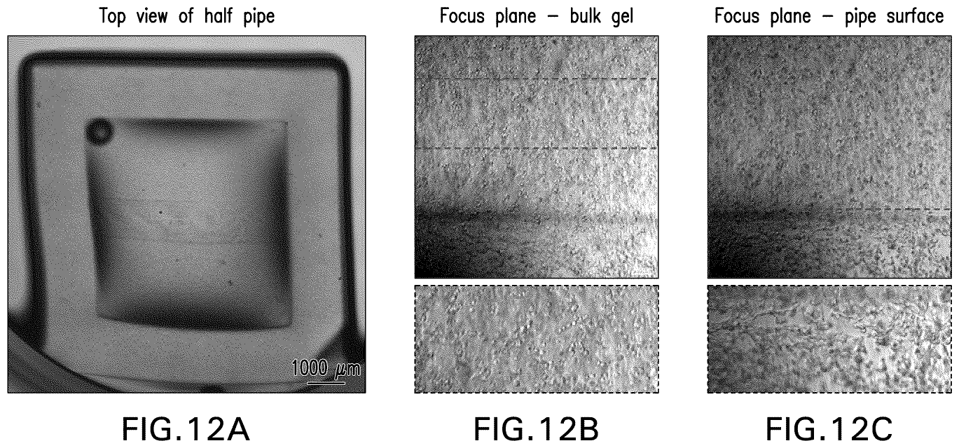

A 2.5D half pipe with astrocytes incorporated into the print solution is shown after one night of soaking Vasculife media (FIG. 12). From the top view (FIG. 12a), it can be seen that astrocytes are incorporated throughout the x and y dimensions of the half pipe print. It can be noted that the astrocytes take on a different morphology in varying z positions of the half pipe. In the printed hydrogel bulk, suspended in 3D, the astrocytes appear to have a balled-up morphology (FIG. 12b). Whereas, when focused on the surfaces of the half pipe, the astrocytes appear to be elongating more substantially (FIG. 12c).

### Tri-Culture of Endothelial Cells, Pericytes, and Astrocytes Results in the Formation of Mature Cell-Cell Junctions

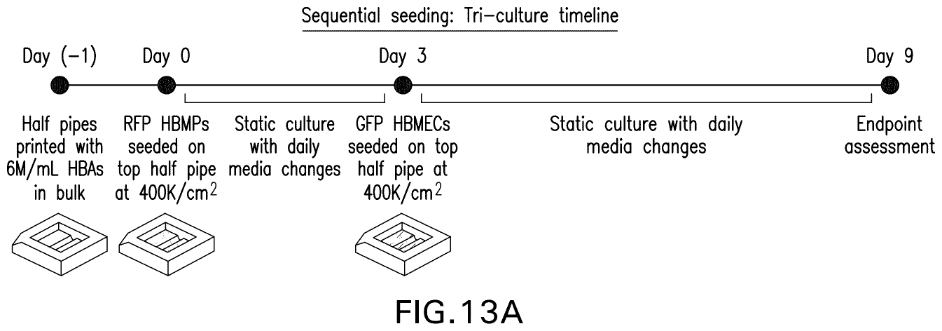

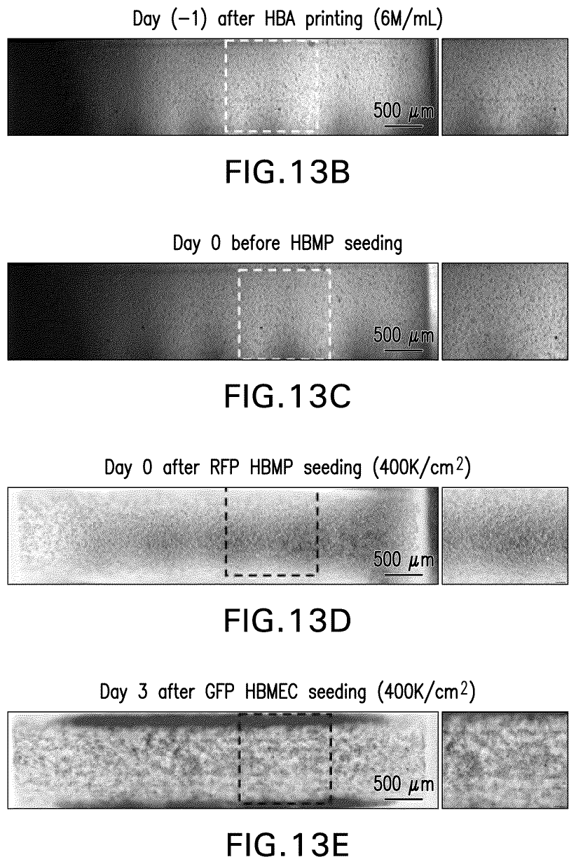

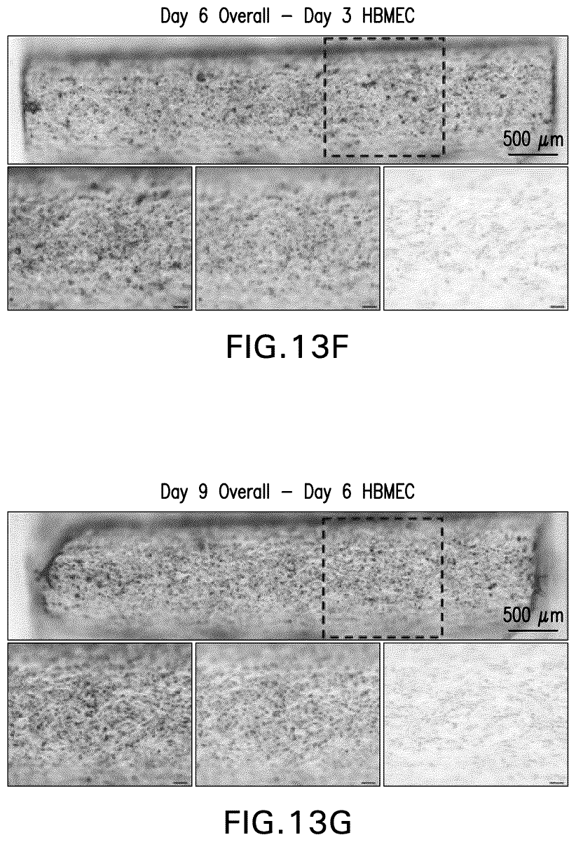

Hydrogel half pipes with astrocytes were printed on day-1 (FIG. 13b) and allowed to soak overnight before the next cell seeding. On day 0 before the pericyte seeding, it was noted that the astrocytes began to take on a more elongated morphology along the surface of the half pipe than had been seen in the day prior (FIG. 13c). The pericytes were seeded onto the half pipe at a density of 400 K cm⁻² (FIG. 13d) and there was homogenous coverage of pericytes across the pipe. After three days of culture, endothelial cells were seeded on top of the astrocyte-printed pericyte-laden half pipes (FIG. 13e). Images were taken daily to assess the progression of morphology for all cell types involved, but images are shown below on day 6 overall and day 9 overall for simplicity (FIGS. 13f & 13g, respectively). From these images, it can be seen that the endothelial and pericyte layer is retained along the surface of the half pipe for the entire duration of the experiment, where the two cells co-exist in the same areas rather than forming gaps in one cell layer. Additionally, the endothelial layer begins to take on a more monolayer-like morphology by day 6 overall (day 3 endothelial cell culture). The pericytes also have a notable morphology progression over the tri-culture duration. Between days 0 and 3, the pericytes spread out on along the surface of the half pipe. Their elongation and covering of the pipe continued for the remainder of the tri-culture timeline.

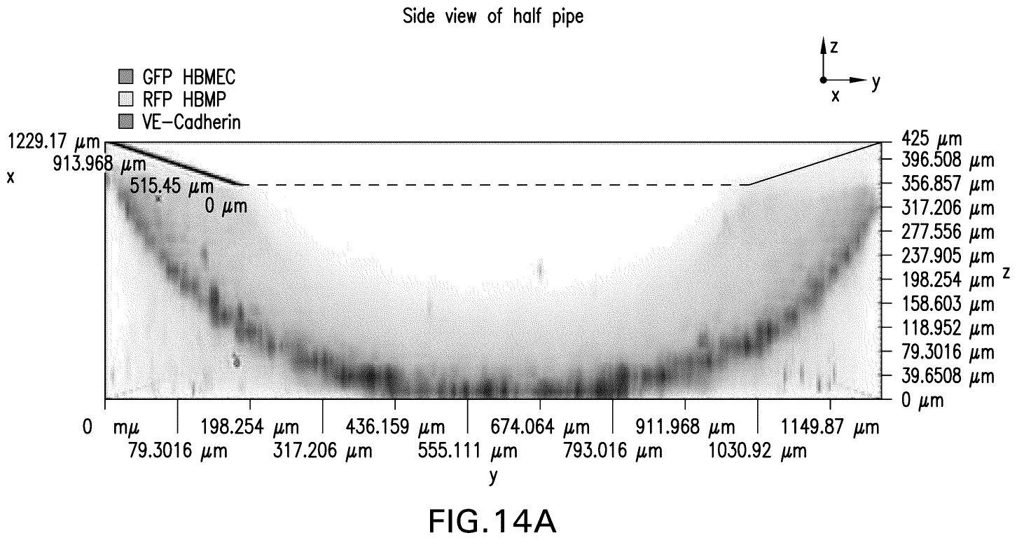

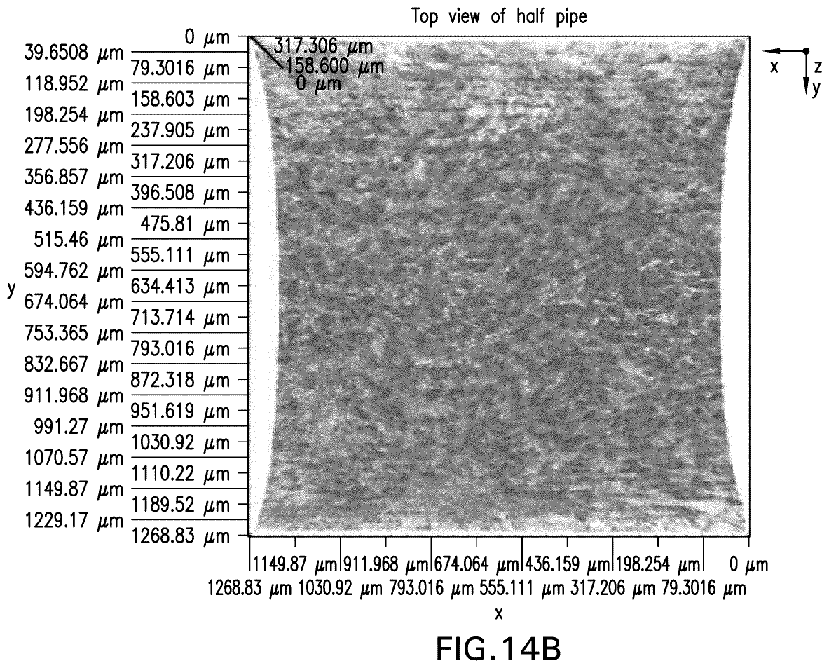

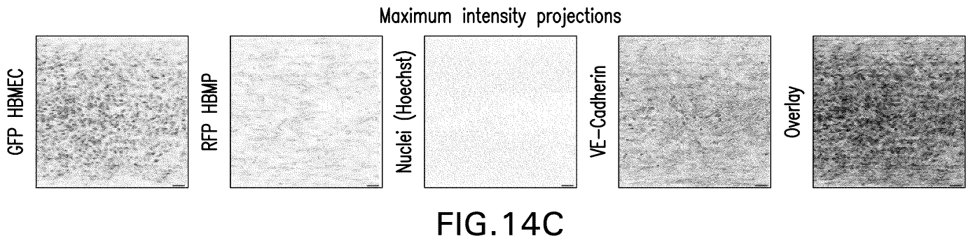

With the fabrication of this tri-culture using the printed half pipe design, it was also important to assess the presence of cell-cell junctions within the model. To do this, the half pipe was immunostained for VE-Cadherin and the presence and morphology of cell-cell junctions was analyzed. A 3D rendering of the half pipe immunostained for VE-Cadherin is shown from the side view (FIG. 14a) and the top view (FIG. 14b). Maximum intensity projections for each split channel are shown in FIG. 14c. From this, the presence of mature cell-cell junctions is demonstrated in this 2.5D tri-culture model.

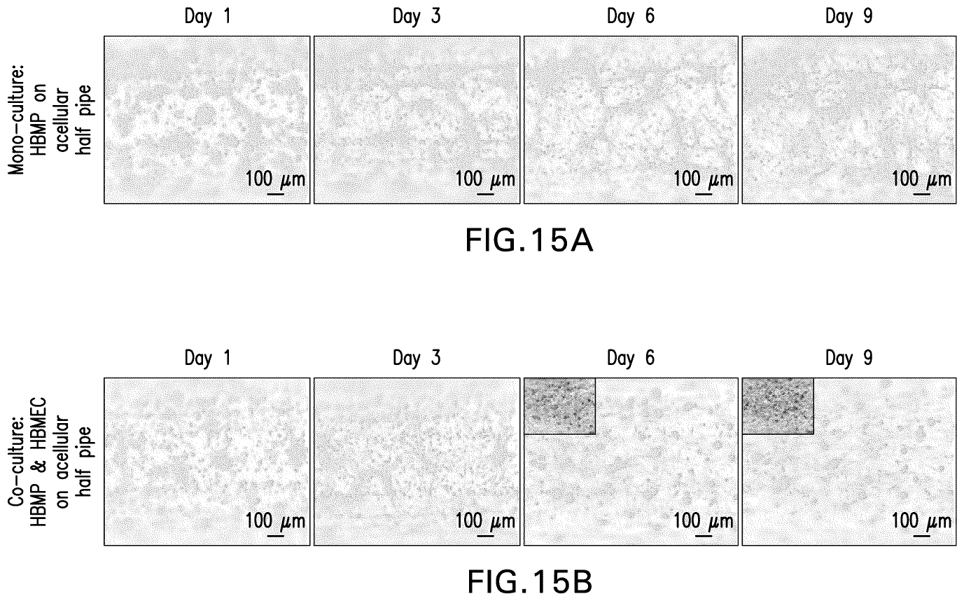

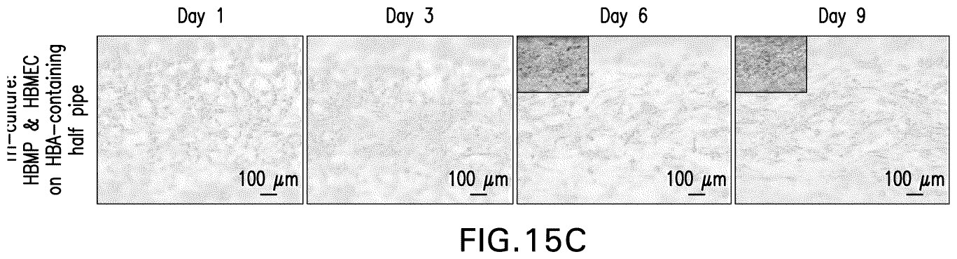

It is well reported in literature that supporting cells of the BBB, specifically pericytes and astrocytes, are essential for vascular maintenance and homeostasis. Below the morphological differences can be seen for each cell type in its respective mono-, co-, and tri-culture condition. Pericyte morphology is shown below in mono-culture (pericytes alone on acellular half pipe, FIG. 15a), co-culture (pericytes and endothelial cells on acellular half pipe, FIG. 15b), and tri-culture (pericytes and endothelial cells on astrocyte-containing half pipe, FIG. 15c). In the mono-culture condition, the pericytes do not demonstrate substantial spreading or adherence throughout the nine days in culture. In the co-culture condition, the pericytes exhibit little spreading between days 1 and 3, but demonstrate a morphology change between days 3 and 6 in the presence of endothelial cells. Once the endothelial cells are added, the pericytes begin to spread and elongate along the printed half pipe. Finally, in the tri-culture condition, there is a noticeable difference with the pericyte adhesion, where the pericytes appear more spread out on day 1 compared to the mono-and co-culture conditions, and the pericytes continue to spread and fill the surface of the half pipe by day 3. One hypothesis for the differential adhesion and spreading seen by the pericytes on the cellular half pipes may be attributed differences in hydrogel stiffness. The cellular gels are softer than their acellular counterpart due to the incorporation of cells in the print solution, which may result in differential spreading between the different gels. It's been reported that pericytes show optimal spreading on intermediate (20-40 kPa) substrate stiffness, while showing less favorable spreading on both softer and stiffer substrates. An additional factor that could be contributing to the pericyte spreading is due to astrocytic secreted factors. Astrocytes have been shown to secrete factors that induce pericyte synthesis of fibronectin. It has also been reported that pericytes strongly prefer fibronectin over laminin for adhesion formation. The production and deposition of fibronectin by the pericytes may be guiding additional adhesion.

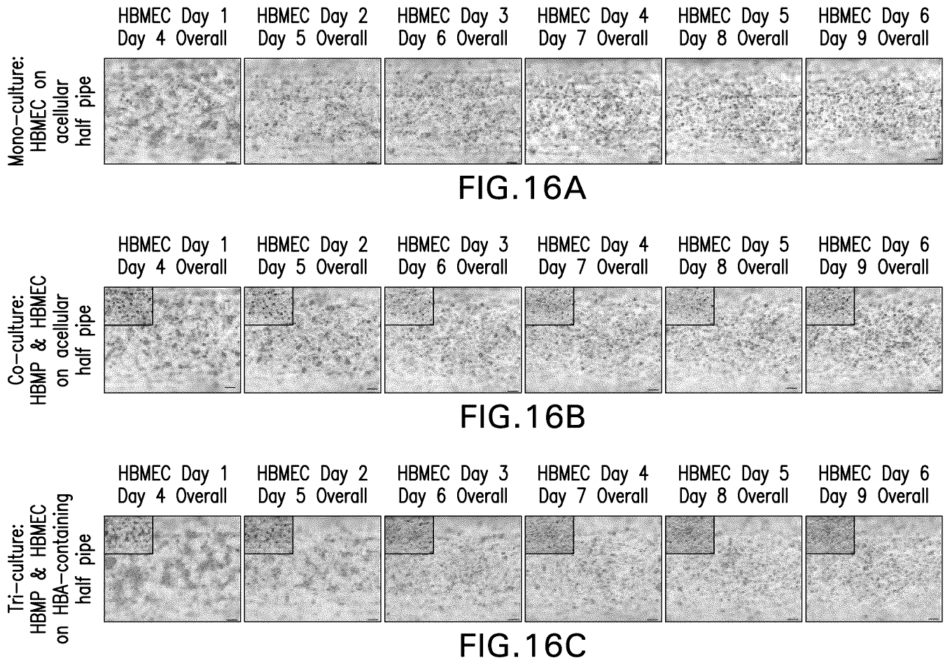

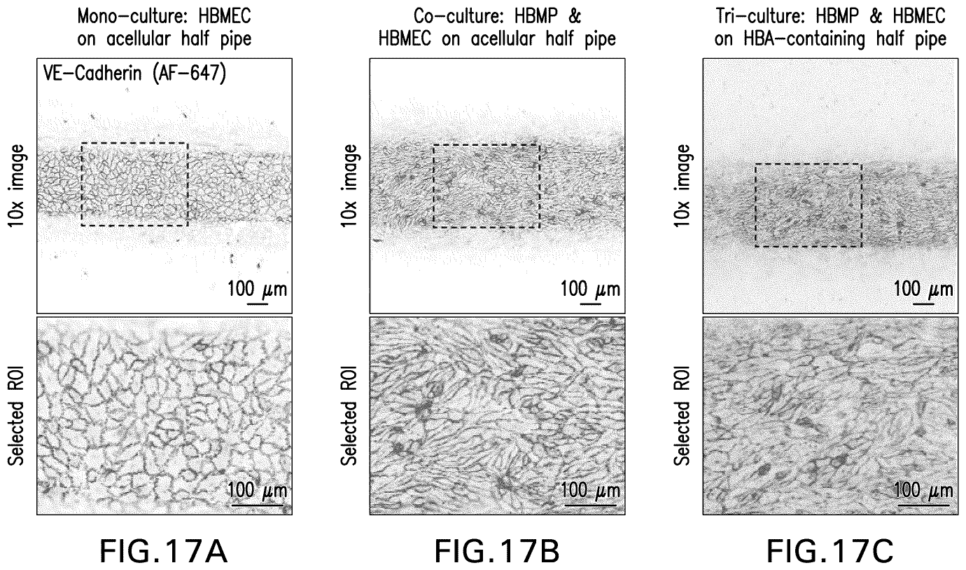

Similarly, the morphological differences for endothelial cells in mono-, co-, and tri-culture conditions can be seen below in FIG. 16. For the mono-culture endothelial cells (FIG. 16a), there is substantial adherence and spreading compared to the pericyte monoculture. It appears that by day 3 of endothelial cell culture, the cells begin to arrange into a more monolayer like structure where the cells begin to organize alongside each other rather than being stacked on top. For the co-culture condition (FIG. 16b), that change appears to take place a bit sooner, where the cobblestone morphology appears prevalent around day 2-3. Finally, for the tri-culture condition (FIG. 16c), there appears to be a high number of cells the first two days of culture, but a cobblestone, monolayer morphology appears visible between days 3-4. While using the cells' endogenous fluorescence provides insights on daily morphology, it is challenging to make definitive assessments without the use of immunostaining.

Immunostaining for the cell-cell junction, VE-Cadherin, was performed for the mono-, co-, and tri-culture conditions for the half pipes assessed (FIG. 17). All three conditions of endothelial cells display strong staining for cell-cell junctions, however, there are some slight differences in morphology. For the mono-culture condition (FIG. 17a) the cells appear to have a more rounded, circular morphology. For both the co-culture (FIG. 17b) and tri-culture (FIG. 17c) condition, the cells demonstrate a more elongated, packed morphology.

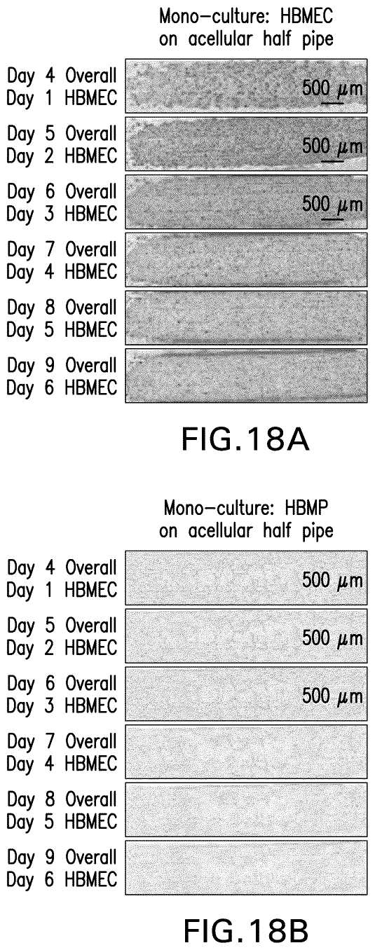

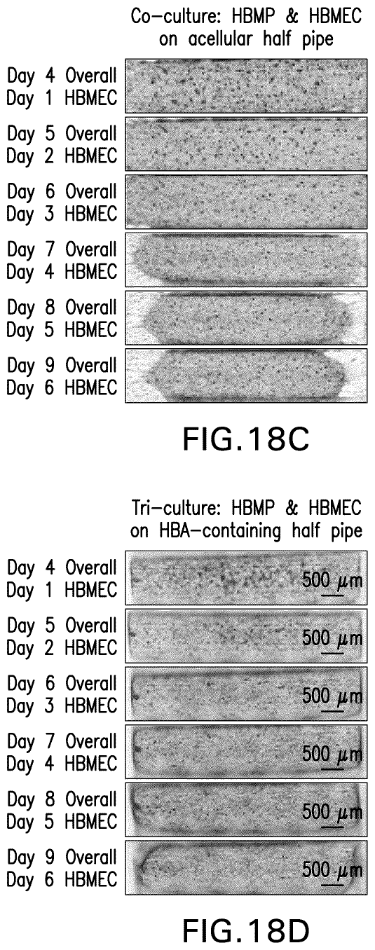

In addition to seeing morphological differences for the cell types between mono-, co-, and tri-culture conditions, there were also differences seen in the retention of the cell layer across the length of the half pipe. For the endothelial monoculture (FIG. 18a) and the pericyte monoculture (FIG. 18b), the cell layer remains intact across the length of the pipe for the entire culture duration. In contrast, for the co-culture condition of pericytes and endothelial cells (FIG. 18c), there is an apparent retraction of the co-culture cell layer that begins on day 7 and persists through the end of the study. However, in the tri-culture condition with astrocytes incorporated into the printed structure with pericytes and endothelial cells cultured on top (FIG. 18d), this cell layer retraction is mitigated, and the retraction is not significant for the duration of the study. This demonstrates that the astrocytes are having an effect on stabilizing the cell layer and play an important role in maintenance of the cellularized layer. This stabilization after astrocyte incorporation could be attributed to the production of astrocytic laminin, as laminin is a key component of the basement membrane. Astrocytes produce laminins-111 and -211, and these astrocyte-specific laminins are only located in the vasculature of the brain, indicating they may play a crucial role for BBB function. It has been demonstrated that a lack of astrocytic laminins induces pericyte differentiation from a resting stage stabilizing the BBB into a contractile stage disrupting the BBB. This pericyte contractile state may be causing the retraction seen in the co-culture model below (FIG. 18c), which is mitigated by the incorporation of astrocytes in the tri-culture (FIG. 18d). To validate this hypothesis, immunostaining against laminins-111 and-211 could be conducted to visualize if astrocytic laminins are present in the tri-culture model.

### Achieving Shorter Tri-Culture Timelines for Accelerated Experimental Iteration

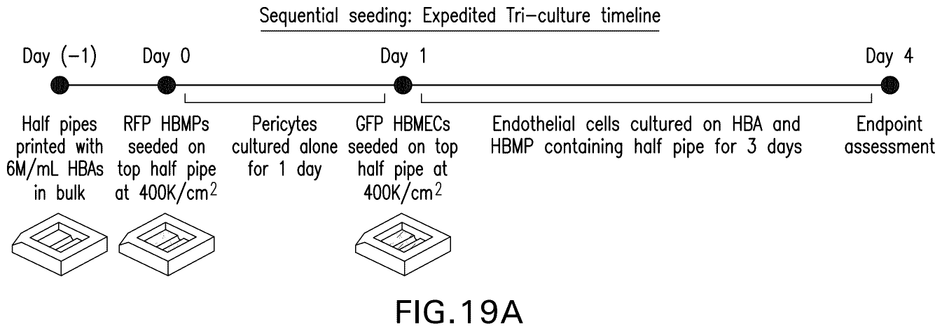

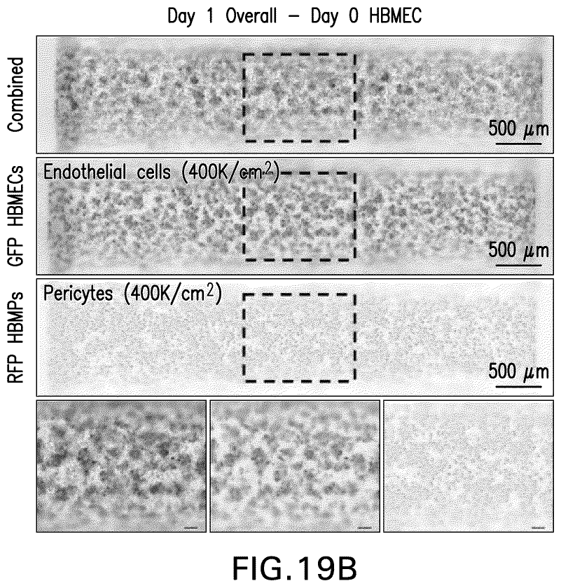

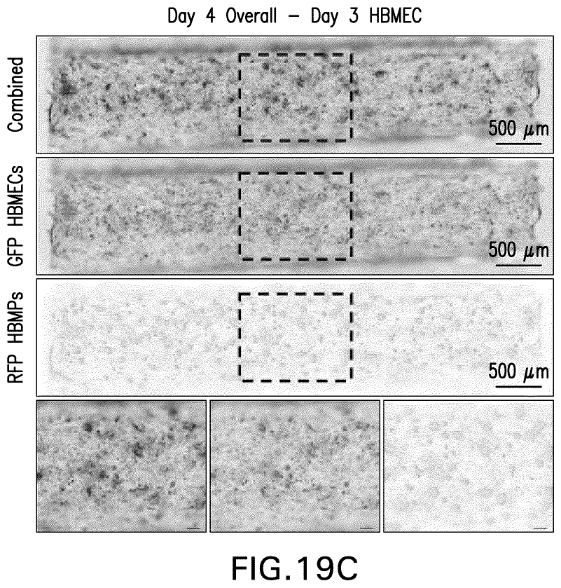

The ability to achieve a tri-culture on this printed gel structure was demonstrated with a total timeline of ten days, including allowing the astrocyte-printed gel to stabilize overnight, providing three days for pericyte culture, and sustaining the endothelial cells for six days after their addition on day 3. While this timeline demonstrated potential and could be used for future studies, this ten-day timeline limits iteration between studies and throughput due to extended culture times. Thus, this half pipe model was also used to assess if the timeline for each culture step could be shortened for an expedited tri-culture timeline to accelerate turnaround time on studies and enable increased throughput in a shorter amount of time. In past studies, it was seemed that minor morphological changes occurred for the pericytes between days 2-3 in tri-culture, which was the rationale for using a 1-day culture period before adding the endothelial cells in this expedited timeline. Additionally, in past tri-culture studies the endothelial monolayer appeared to have the highest coverage with the characteristic cobblestone morphology around days 3-4 in tri-culture, providing the rationale for the 3-day culture period before endpoint assessment in this accelerated timeline. Thus, the expedited tri-culture timeline comprises printing the hydrogel structure with astrocytes and allowing an overnight soak, seeding the pericytes on the printed structure on day 0, then seeding the endothelial cells on top on day 1 and allowing to culture for three days after, for a total tri-culture timeline of four days (FIG. 19a). In doing this, both pericytes and endothelial cells had high coverage on day 1 overall (day 0 endothelial cell culture), shown in FIG. 19b. After three days of culture, the endothelial and pericyte layer was sustained on the astrocyte containing gel with the layer covering the entire surface of the pipe (FIG. 19c). Additionally, the pericytes took on the desired elongated morphology, while the endothelial cells appeared to be tightly packed with a cobblestone morphology.

### Presence of BBB Efflux Transporters and Receptor-Mediated Transporters Confirmed in Hemi-Cylinder Model

One of the most important transporters at the BBB is P-gp. P-gp is encoded by MDR1/ABCB1 and is part of the ATP-binding cassette (ABC) transporters. The human genome contains 49 genes that encode ABC transporters, and those are divided into seven subfamilies (A-G). The gene that encodes P-gp is classified within the ABCB subfamily of the ABC transporters. The ATP-binding cassette genes are the largest family of transmembrane proteins; they function by binding ATP and using that energy to drive the transport of molecules across the cell membrane.

P-gp is an efflux transporter, present on the luminal membrane of BBB endothelial cells, that plays a role in the transport of several compounds across the BBB P-gp is located throughout the body in organs and tissues with either excretory functions (such as the liver, kidney, and small intestine), as well as those with blood-tissue barriers (such as the BBB, blood-testis barrier, and placenta). When a substance from the blood attempts to pass through the luminal membrane of brain endothelial cells, it is expelled back into the lumen by embedded efflux transporters, one notable transporter being P-gp. While this is an important function for maintaining brain homeostasis and ensuring harmful compounds in the bloodstream do not cross over into brain tissue, it also poses a challenge for delivering therapeutics from the bloodstream across the BBB, as P-gp acts to expel these compounds back into the bloodstream once they enter the cell membrane. As a result, many therapeutic strategies have aimed to inhibit P-gp expression in order to enable the transport of therapeutics across the BBB.

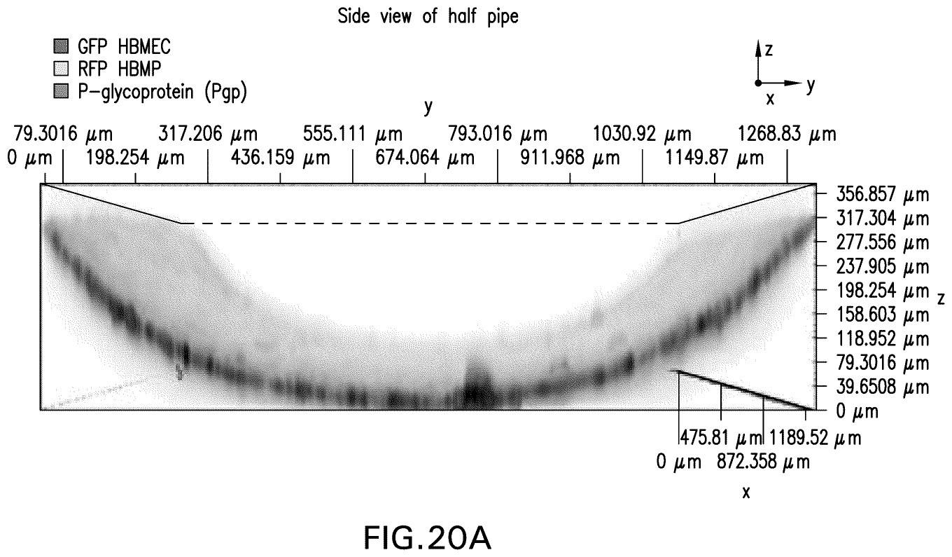

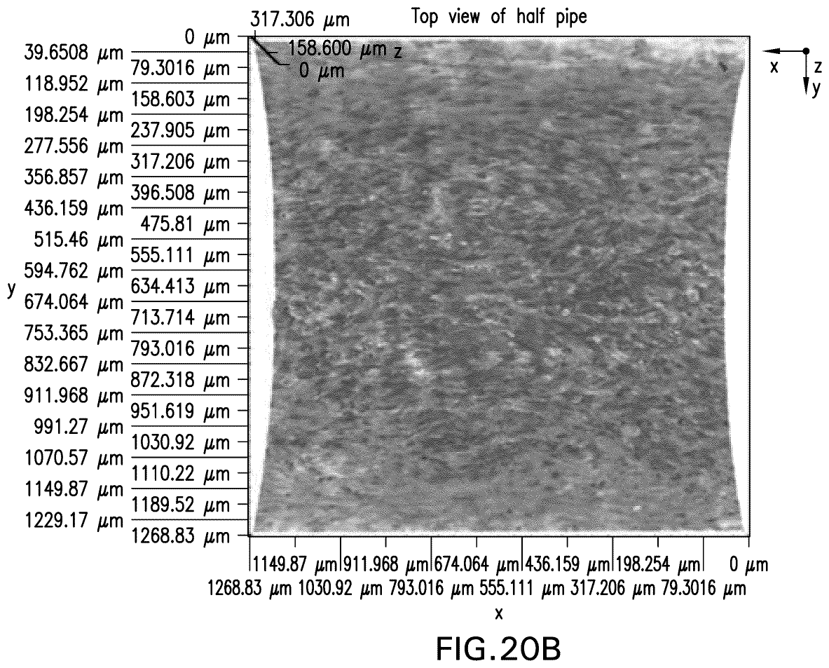

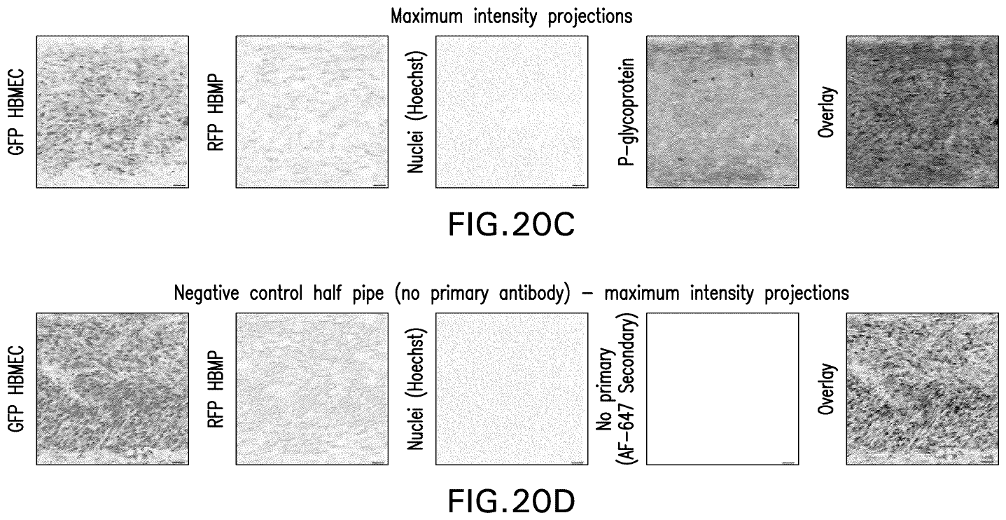

In this model, the presence of P-gp was confirmed with immunostaining on the 2.5D half pipe, as seen in FIG. 20. Demonstrating the presence of this efflux transporter within the hydrogel model displays sufficient complexity for use as a screening tool to assess therapeutic strategies that operate via P-gp inhibition. One particular area where P-gp inhibition is used as a tool is for the delivery of anticancer drugs. P-gp in the BBB restricts the entry of many anticancer drugs from the bloodstream to the brain. Inhibition of this efflux transporter has shown promise as an option for delivery of anticancer drugs to brain tumors via the co-administration of anticancer drugs and a P-gp inhibitor. In the translation to the 3D printed perfusable hydrogel model, this delivery route could be studied via the perfusion of an anticancer compound with a P-gp inhibitor and quantifying the amount of anticancer compound that has crossed the cellular barrier.

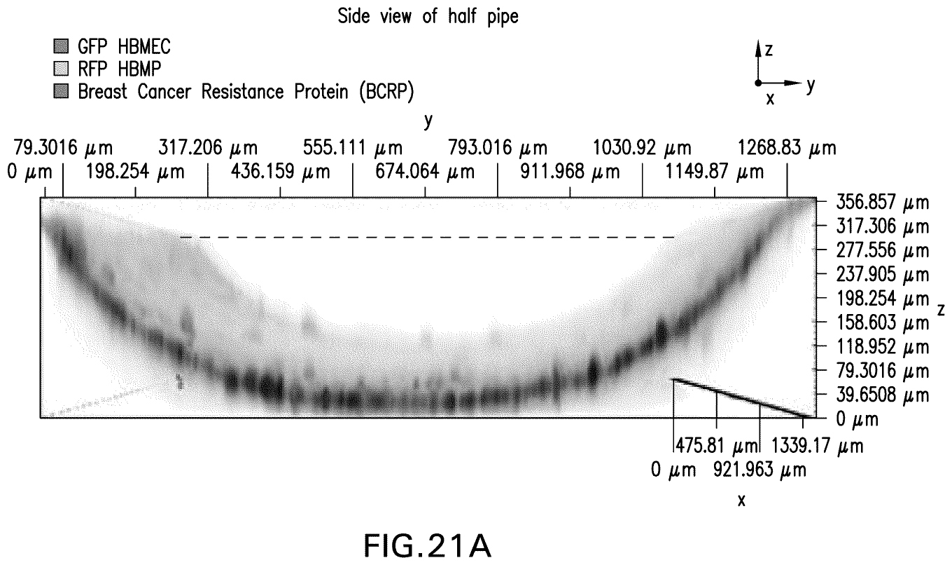

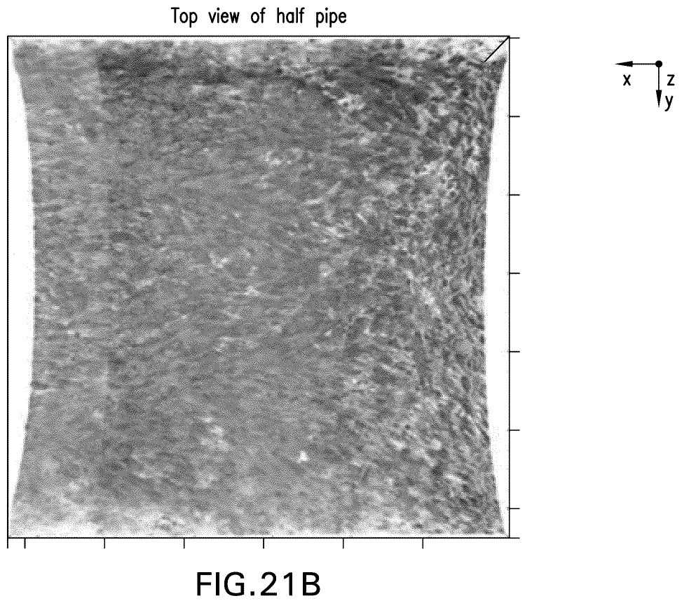

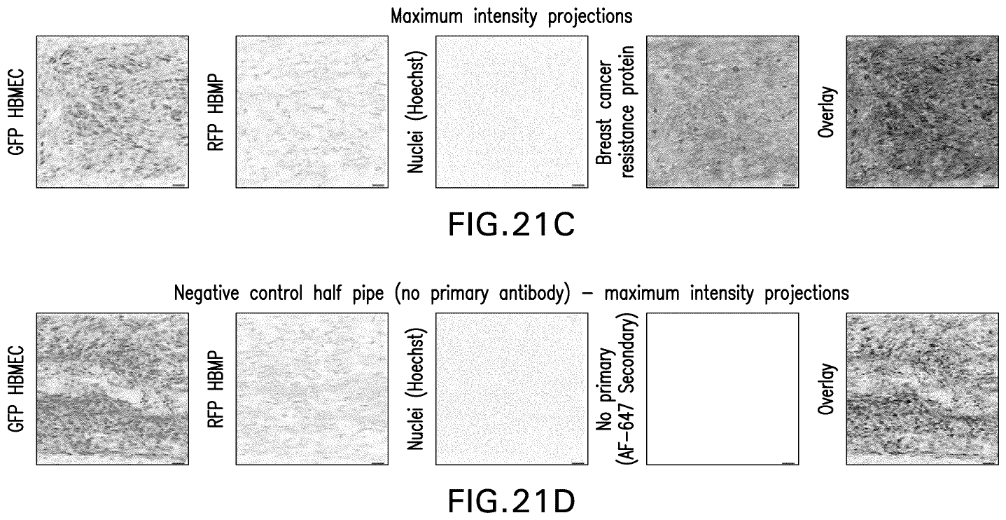

The next transporter assessed in this model was the BCRP. BCRP, like P-gp, is a drug efflux ABC transporter principally expressed on the luminal membrane of brain endothelial cells. BCRP belongs to the ABCG subfamily and is also referred to as ABCG2. For localization and tissue distribution, BCRP is found in similar areas to the other drug efflux transporters, such as P-gp, so it is expected that this protein plays a similar transport role, both in physiology and pharmacology. Historically, most focus was given towards studying P-gp as the main efflux pump of the central nervous system, and it wasn't until later that the role of BCRP was examined further for its role in protecting the central nervous system. Within the human BBB, it has been demonstrated that P-gp and BCRP are the main ABC transporter genes expressed in brain microvessels. As a result, many strategies based on efflux pump inhibition are focused on P-gp and BCRP. In the model displayed here, the presence of the efflux transporter, BCRP, is confirmed via the use of immunostaining (FIG. 21). This taken together with the presence of P-gp demonstrate that this model is suitable for utility in screening therapeutic strategies that work via efflux pump inhibition.

The next transporter that was assessed is the transferrin receptor. Brain capillary endothelial cells have several unique properties, one being that these cells express receptors and transporters that play a role in the uptake of molecules from the bloodstream. These transporters are utilized for transporting biologics to the brain parenchyma via receptor-mediated transport. Due to the strong tight junctions that exist between brain endothelial cells, alternative routes must be devised for the delivery of therapeutic proteins with high molecular weights to the brain. One route to achieve the delivery of higher molecular weight molecules is utilizing receptor-mediated transport. At the BBB, RMT occurs via several steps. In the first step, receptor-mediated endocytosis of a compound occurs when a ligand from the bloodstream binds a receptor on the luminal side of the plasma membrane. Next, the intracellular vesicle that is formed and contains the receptor-ligand complex is transported through the cytoplasm of the endothelial cell. Finally, exocytosis of the drug/transported molecule occurs at the abluminal side of the brain capillary endothelium and the vesicle contents are released into the brain parenchyma.

Receptor-mediated transport is an exciting route to explore for drug delivery, as it enables the transport of large molecule drugs across the BBB. One prevalent method that is used for receptor-mediated transport is the use of antibodies against the transferrin receptor. Iron is essential to the cells of the brain, so the endothelial cells contain a special mechanism to ensure the transport of iron across their restrictive barrier. The iron in blood serum is complexed with the iron-binding protein transferrin. Transferrin is a glycoprotein (molecular weight: 80,000) whose primary function is the transport of iron throughout the body, as iron is unable to travel by itself. When the complex of iron and transferrin reaches a TfR, the transferrin with iron bins to the receptor and endocytosis occurs, forming a vesicle that carries the bound transferrin and iron into the cell.

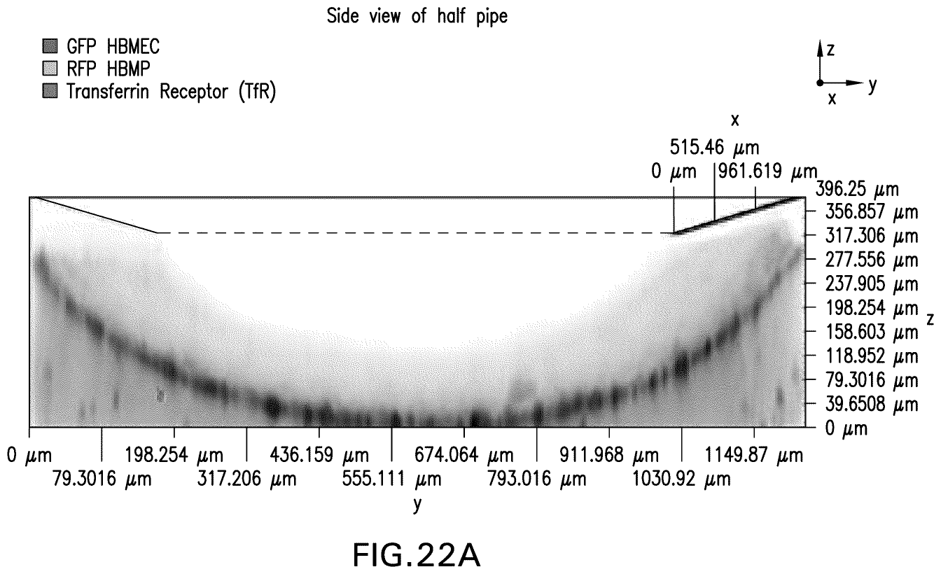

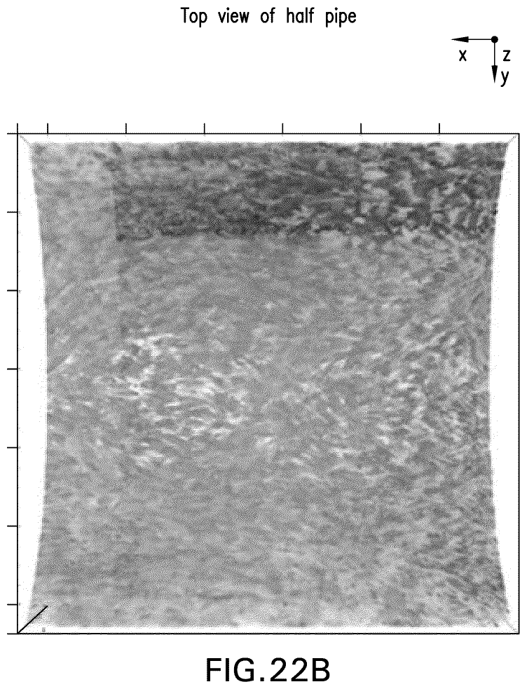

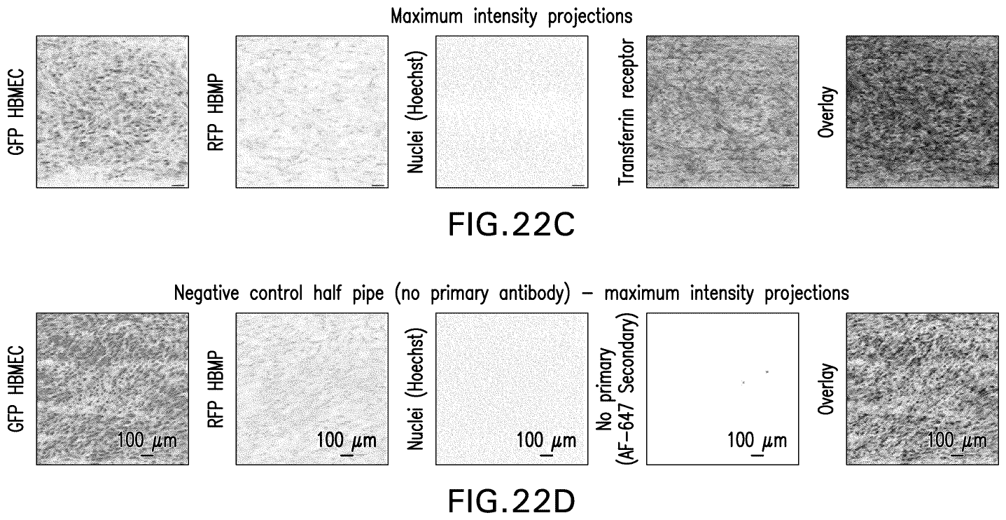

One strategy that is used for brain delivery of high molecular weight therapeutic proteins is the molecular Trojan horse (MTH) method, which delivers therapeutic proteins into the brain via receptor-mediated endocytosis and transcytosis. The most common receptors expressed on the luminal side of brain endothelial cells that are used for the molecular Trojan horse method are the insulin receptor and the transferrin receptor. In this method, a therapeutic protein drug is fused to an antibody that binds to a specific receptor on the endothelial cells of the blood brain barrier. This approach enables the receptor-mediated delivery of the fusion protein across the restrictive BBB and allows for delivery of the therapeutic protein to the brain. In this model, the presence of the TfR was confirmed with immunostaining on the 2.5D half pipe, as seen in FIG. 22. This shows promise that this model can be used as an in vitro screening tool for therapeutic strategies that operate via the molecular Trojan horse method for the delivery of large molecule therapeutics.

The final transporter assessed for in this model was low density LRP-1. Similar to the drug delivery approach utilizing the transferrin receptor, delivery approaches involving LRP-1 also rely on RMT and taking advantage of the highly expressed, endogenous receptors and mechanisms present at the endothelial cells of brain capillaries. Due to the extensive capillary network and perfusion rate within the brain, using transporters for receptor-mediated transport is considered one of the most effective methods to delivery therapeutic drugs to the brain parenchyma and is recognized as one of the methods with the most likely chance of success.

LRP-1 plays an essential role in the cellular transport of cholesterol, endocytosis of multiple ligands, and transport of ligands across the BBB. Within the central nervous system, LRP-1 is expressed in neurons, glial cells, and vascular cells and plays an essential role in the maintenance of brain homeostasis. Some of the ligands that interact with LRP-1 include apolipoprotein E, tissue plasminogen activator, blood coagulation factors, receptor-associated protein, Alzheimer's disease amyloid beta-peptide, aprotinin, and many others.

There are several receptors in the low-density lipoprotein receptor family, however, LRP-1 is the most studied receptor within this family due to its critical role in multiple pathways in the pathogenesis of Alzheimer's Disease. Within the last decade, evidence has emerged that suggests that LRP-1 is involved in regulating the brain and systemic clearance of Alzheimer's disease amyloid beta-peptide (A-beta) via transcytosis through the brain endothelium for systemic elimination and it has been suggested that impairment of LRP-1 contributes to the accumulation of amyloid-beta and drives Alzheimer's disease pathology. LRP-1 has roles in both the generation and clearance of amyloid-beta; the endocytosis of amyloid precursor protein (mediated by LRP-1) is necessary for generation of amyloid-beta within the cells, however, LRP-1 is also involved in the clearance of extracellular amyloid-beta.

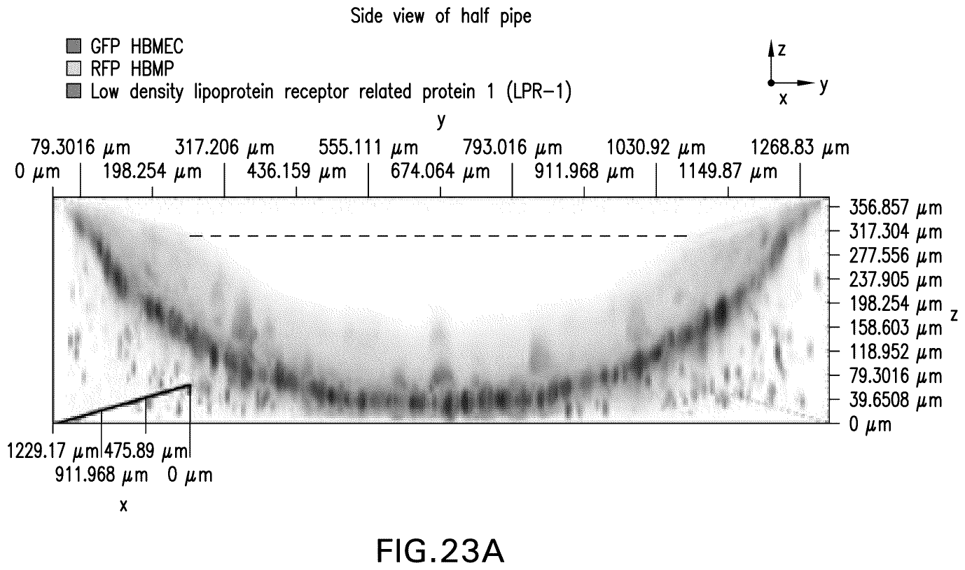

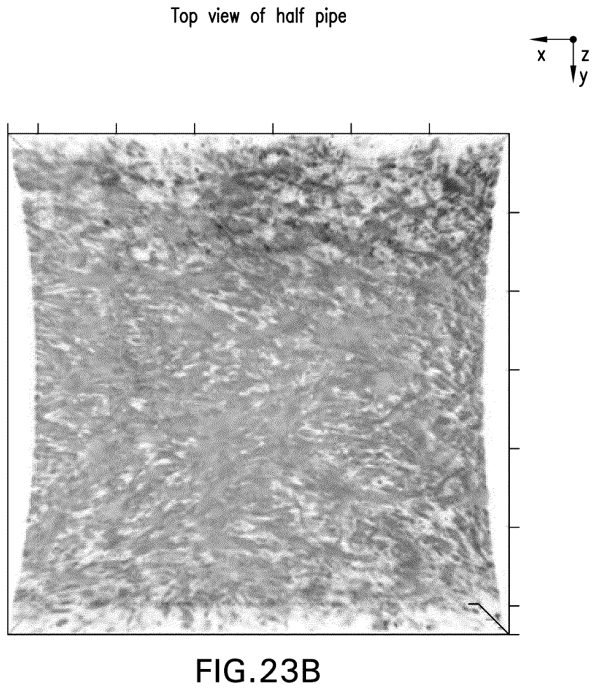

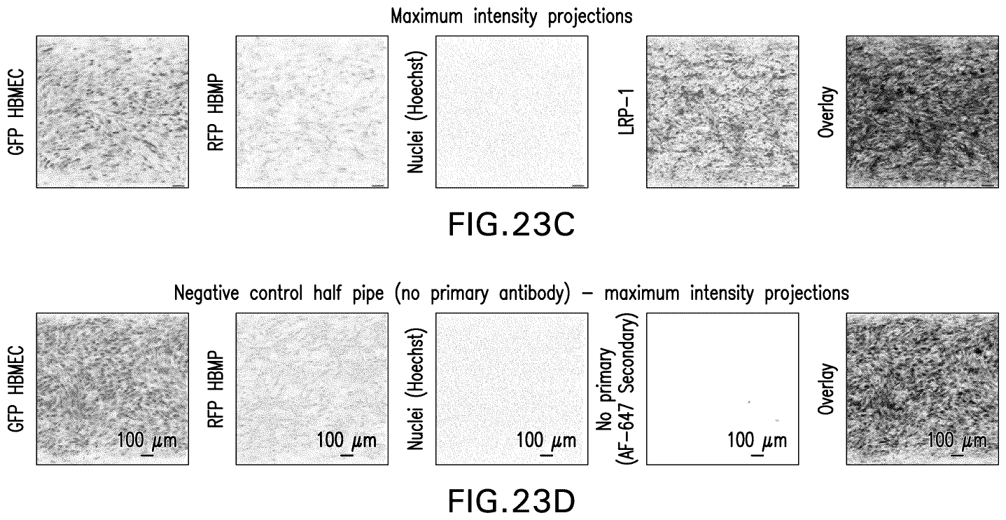

In addition to being involved in the pathology of Alzheimer's, understanding LRP-1 and its transport mechanism is also of interest for other pathologies, such as glioblastoma. Glioblastoma is extremely difficult to treat, one reason being the presence of restrictive barriers such as the BBB and the blood-brain tumor barrier, thus underscoring the need for drug delivery strategies. LRP-1 is widely expressed in the BBB, as well as in glioblastoma. One of the ligands that LRP-1 interacts with, receptor-associated protein (RAP), has the ability to bind to bind to LRP-1 and internalize into the endothelial cells with these receptors via receptor-mediated transport. Additionally, receptor-associated protein has been shown to cross the BBB with higher efficiency than transferrin, demonstrating promise as an effective brain delivery tool. The presence of LRP-1 in this hydrogel model, seen in FIG. 23, demonstrates that this in vitro model has the potential to be used as a screening tool to study both drug delivery approaches, as well as pathologies associated with the LRP-1 transporter.

### BBB Model Translation to 3D Printed Perfusable Channel

While the 2.5D hydrogel half pipe model was extremely useful for determining crucial parameters for the tri-culture model in a high-throughput manner, it lacks key physical forces, namely shear stress, that play an important role in endothelial barrier function. Thus, the next step in this work was to extend the model parameters that were determined in 2.5D into a 3D perfusable model. This was approached in a step-wise fashion of first assessing human brain microvascular endothelial cell (HBMEC) behavior in an acellular serpentine hydrogel, then investigating the sequential co-culture of human brain endothelial cells (HBMECs) and human brain microvascular pericytes (HBMPs) seeded in an acellular serpentine hydrogel. Finally, the work culminates with the incorporation of all three cell types into this 3D printed perfusable model where human brain astrocytes are printed in the bulk of a perfusable hydrogel, followed by sequential seeding of pericytes and endothelial cells in the vascular channel.

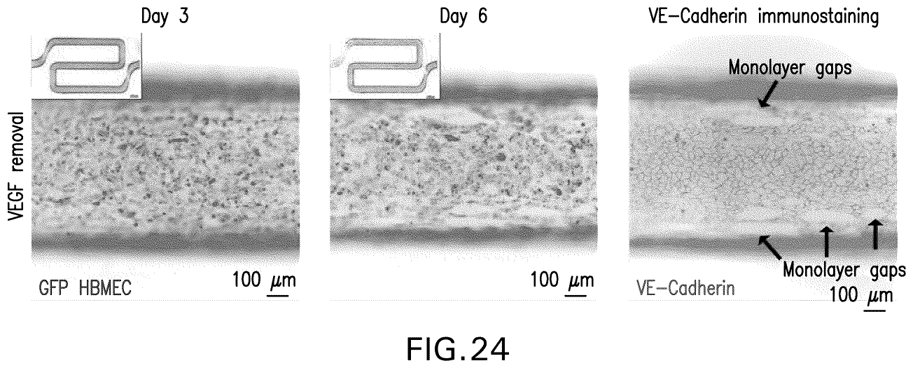

In the first studies assessing human brain microvascular endothelial cell behavior, there were differences found between the behavior of the human brain microvascular endothelial cells and the human umbilical vein endothelial cells which much of our work has employed. The ability to retain a complete monolayer with the HBMECs over six days in perfusion culture proved to be non-trivial. The standard media protocol using Complete Vasculife media resulted in the elongated morphology with poor coverage, as seen in HUVECs and as expected. However, the improved media protocol using Complete Vasculife media for three days and switching to a non-VEGF containing Vasculife for the remaining three days of culture was not as efficient in achieving a confluent monolayer as it was with the HUVECs. In the protocol, there were visible gaps present within the monolayer seen after VE-Cadherin immunostaining (FIG. 24). One hypothesis for the differing response of these two lines of endothelial cells could involve the shear stress levels needed in perfusion culture for formation and maintenance of a mature monolayer over the culture period duration. The shear stress reported in veins, relevant to HUVECs, ranges from 1-4 dyn cm⁻², while the shear stress values reported in the capillaries, relevant to HBMECs, range from 10-20 dyn cm⁻². Thus, one strategy to achieve a mature monolayer of HBMECs may employ increasing the flow rate to provide more representative physiologic shear. In any case, other strategies needed to be employed to achieve a confluent monolayer with stable endothelial cell-cell junctions for the brain microvascular endothelial cells.

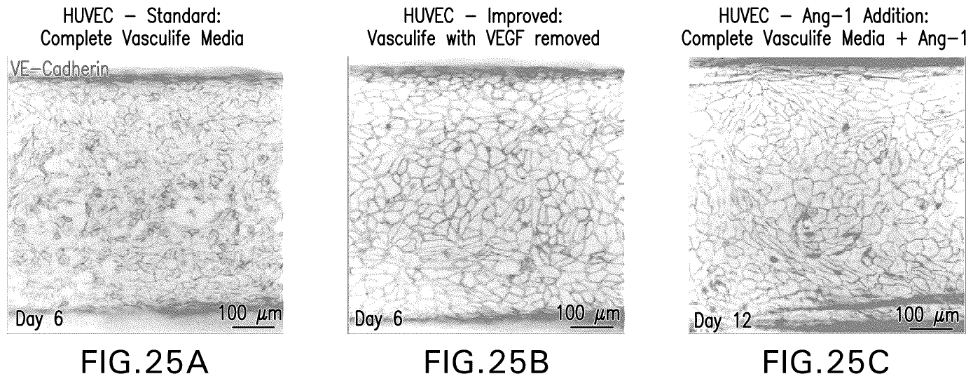

For the HUVECs, a mature monolayer was able to be obtained through modulation of the media composition and utilizing biomolecules that play an important role in endothelial cell function. In addition to the removal of VEGF, another avenue explored was the addition of Angiopoetin-1 (Ang-1). Angiopoetin-1 is a glycoprotein that is a known ligand for the endothelial cell-specific receptor tyrosine kinase, Tie-2 receptor. In vascular development during embryogenesis, VEGF and Ang-1 play essential and complementary roles. Vascular endothelial growth factor (VEGF) is responsible for the initial formation of the vascular plexus, while Angiopoetin-1 (Ang-1) is responsible for the subsequent remodeling of the vasculature into a network of mature vessels. It has been reported that Ang-1 has the ability to block VEGF-induced endothelial permeability in vitro and has been shown to play a role in vessel stabilization even in the absence of mural supporting cells. The incorporation of this glycoprotein was first assessed using hydrogels endothelialized with GFP HUVECs. Three different media strategies were employed to assess the effect of adding Ang-1 to the perfusion media. The first strategy was perfusion for six days with complete Vasculife media, the second strategy was perfusion for three days with complete Vasculife media, then switching to a non-VEGF containing Vasculife (improved protocol, or “no VEGF”), and the third strategy was perfusion for three days with complete Vasculife media, then switching to a Vasculife media with Ang-1 added in for the remainder of the timeline. In the condition where Ang-1 was added in, the hydrogel retained an endothelial monolayer for twelve days of perfusion culture. VE-Cadherin immunostaining for the three conditions was conducted to assess the monolayer morphology (FIG. 25). This strategy for the addition of Ang-1 may play a helpful role where media composition cannot be tuned, or in multi-cellular constructs where supporting cells produce additional VEGF.

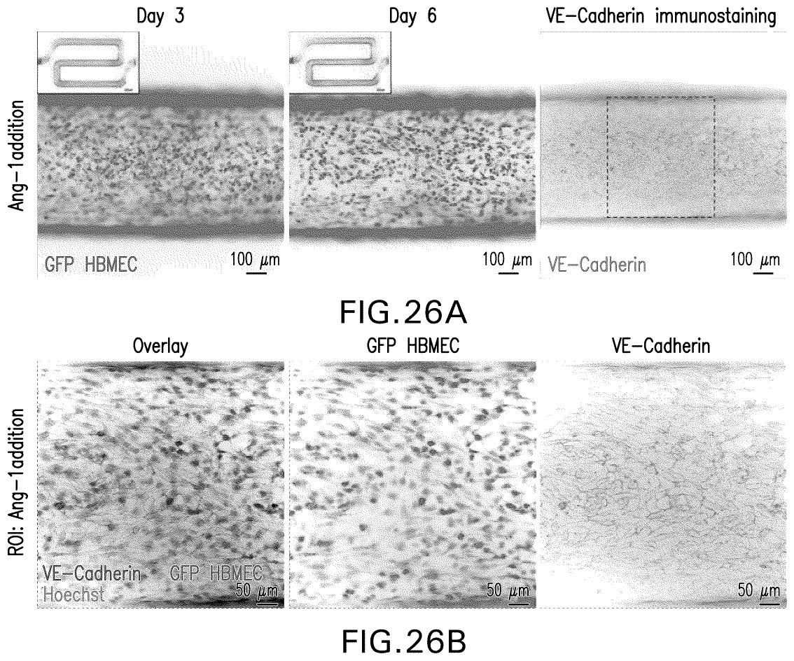

Once this was confirmed using HUVECs, the incorporation of Ang-1 to the perfusion media was tested on a gel endothelialized with GFP HBMECs. The same strategy was employed where the gel was perfused for 3 days with complete Vasculife media, then switched to a Vasculife media where Ang-1 was added in at 15 ng/ml for the remaining three days of perfusion culture. Images of cell morphology in the serpentine on days 3, 6, and after fixing and immunostaining for VE-Cadherin are seen in FIG. 26a. The VE-Cadherin immunostaining demonstrates that a confluent endothelium of brain microvascular endothelial cells lining the serpentine channel has been achieved. A magnified portion of the center channel is shown in FIG. 26b, which highlights the morphology of the GFP HBMECs and VE-Cadherin and includes nuclei in the overlay.

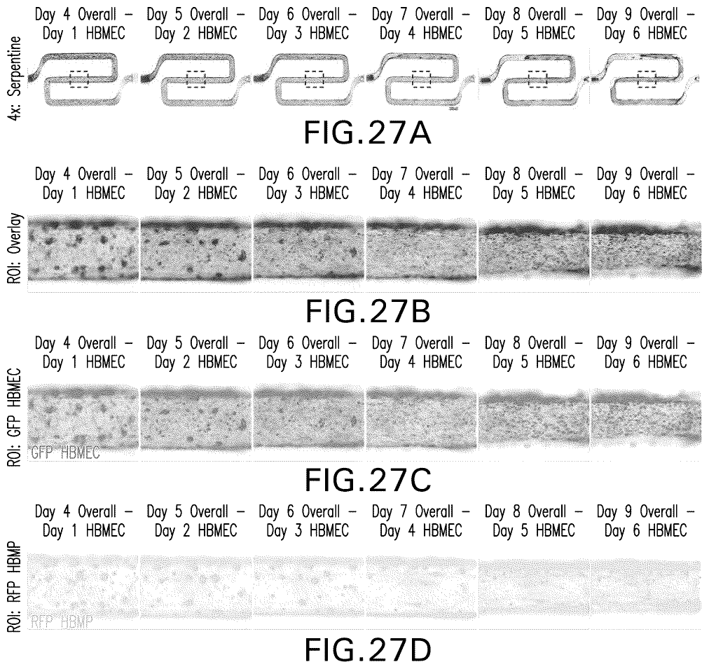

The next step in working with the 3D perfusable model was to examine the co-culture of human brain microvascular endothelial cells and human brain pericytes in the channel. In doing this, the pericytes were seeded into an acellular printed serpentine on day 0, and endothelial cells were seeded on day 3. The progression of co-culture morphology, shown in FIG. 27, enables visualization of the co-culture cell layer throughout the entire vessel architecture (FIG. 27a), a selected ROI in the center of the center channel as an overlay (FIG. 27b), GFP channel only to visualize endothelial coverage and morphology (FIG. 27c), and mCherry channel only to visualize pericyte coverage and morphology (FIG. 27d). In the center ROI, it appears that both endothelial cells and pericytes spread out along the gel surface and take on the morphologies that were seen in 2.5D over time, i.e., cobblestone for the endothelial cell and flattened/elongated for the pericytes. Another interesting thing to note is the detachment of the cell layer that becomes noticeable in the top left corner of the serpentine around day 7, as well as in the center channel ROI around days 8-9. This retraction is mimicking the cell behavior that was seen in the 2.5D studies, providing confidence that the findings in those studies translate well to 3D. Additionally, the retraction of the cell layer seen in 2.5D was mitigated after the incorporation of astrocytes.

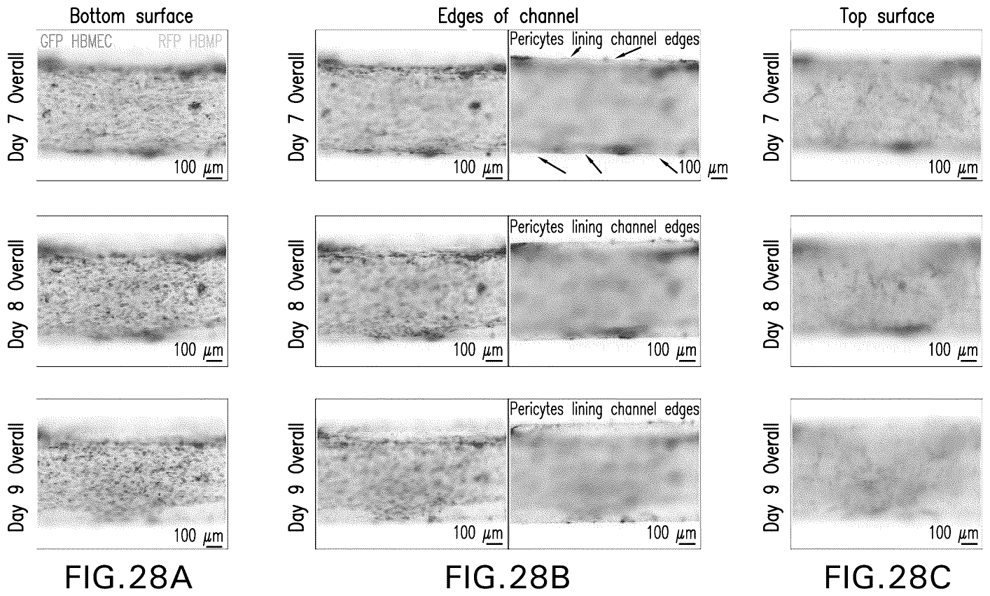

A morphology that was noted in the co-culture serpentine was the wrapping of pericytes along the edges of the channel. FIG. 28 displays different z-positions of a z-stack through the entire channel going from the bottom surface of the channel (FIG. 28a), up through the edges at two z-positions (FIG. 28b) and ending with the top surface of the channel (FIG. 28c). This is interesting to note, as this is representative of the physiologic arrangement of pericytes and endothelial cells, where pericytes are wrapped around the abluminal surface of the endothelial cells. Future evaluation of the arrangement of endothelial cells, pericytes, and astrocytes will play enable the determination of cellular organization and if it is recapitulating the native BBB organization.

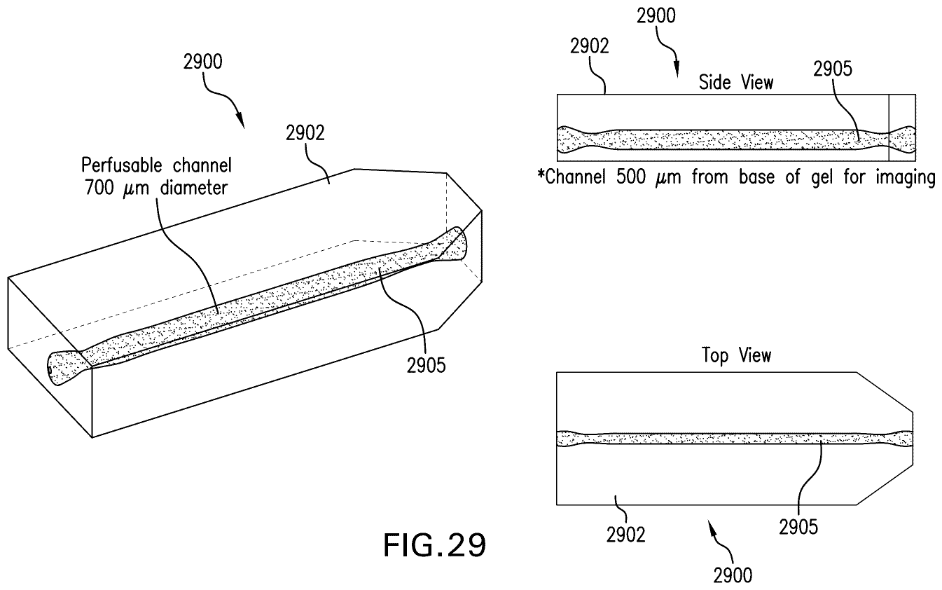

While the serpentine architecture was utilized until this point, a swap was made to leverage a more simple, straight channel design with smaller hydrogel dimensions, as seen in FIG. 29. In this example, a device 2900 includes a hydrogel 2902 of approximately 3 mm in height, 6 mm in width and 16 mm in length that defines a substantially straight channel 2905 with a 700 μm diameter. Channel 2905 may be defined approximately 500 μm from the base of hydrogel 2902 as best seen in the side view of FIG. 29. It will be understood that slight deviations in the channel diameter are possible. The motivation for this switch was to implement a design that minimized hydrogel precursor print solution, thus reducing astrocyte use per gel, since astrocytes are incorporated into the bulk print solution at a density of 6M cells mL⁻¹. This straight channel hydrogel design can be fabricated with three times less hydrogel precursor print solution per print construct, enabling more studies to be conducted while using the same number of astrocytes. While the straight channel was used for early tri-culture 3D studies, the work of this example implemented the serpentine design again for the ultrasound-permeability studies.

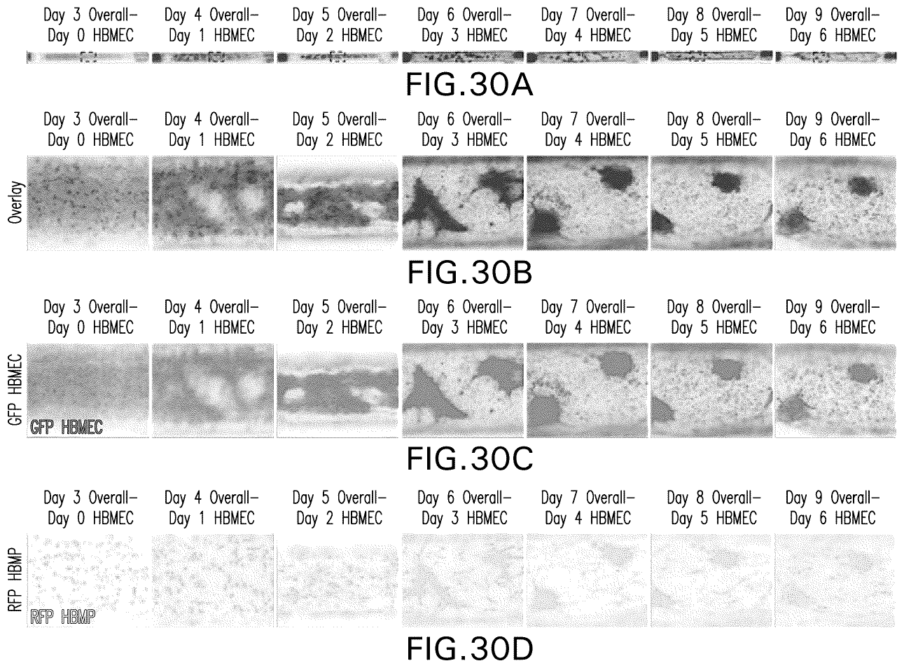

The final studies of this work implemented the sequential seeding method in combination with printing cells into the bulk where unlabeled human brain astrocytes were printed in the bulk surrounding gel, human brain microvascular pericytes were seeded into the channel on day 1, and human brain microvascular endothelial cells were seeded into the channel on day 3. The progression of morphology for both the endothelial cells and pericytes can be seen in FIG. 30. It was noted that the endothelial cells formed large nodules of cells immediately after seeding that persisted throughout the majority of the culture timeline, but noticeable diminished in size as time progressed.

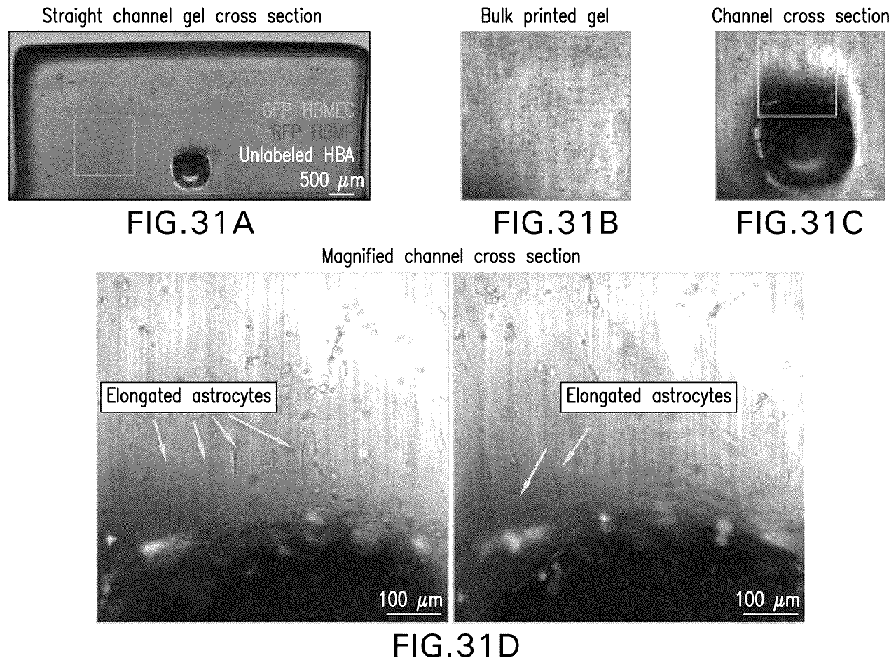

After observing the morphology and cell coverage of the tri-culture system in the channel overtime, it was then cross-sectioned to assess the cellular arrangement of the channel cross-section. The cross section of the gel can be seen in FIG. 31a, with two ROIs outlined to further assess the morphology of the cells at different locations within the gel. In FIG. 31b, the morphology of the bulk printed astrocytes appears to be very rounded, analogous to the morphology seen in the bulk printed astrocytes for the 2.5D half pipe. It is possible that the print formulation is too stiff to enable the astrocytes the ability to spread and reorganize in the bulk matrix. Future work could examine alternative, softer print formulations that would enable this rearrangement. FIGS. 31c & 31d show a portion of the channel cross section where it appears there are astrocytes elongating towards the channel. This shows promise, as the physiologic arrangement of cells in the BBB has astrocytes reaching towards the vessel and having their astrocytic endfeet directly surrounding the vessel.

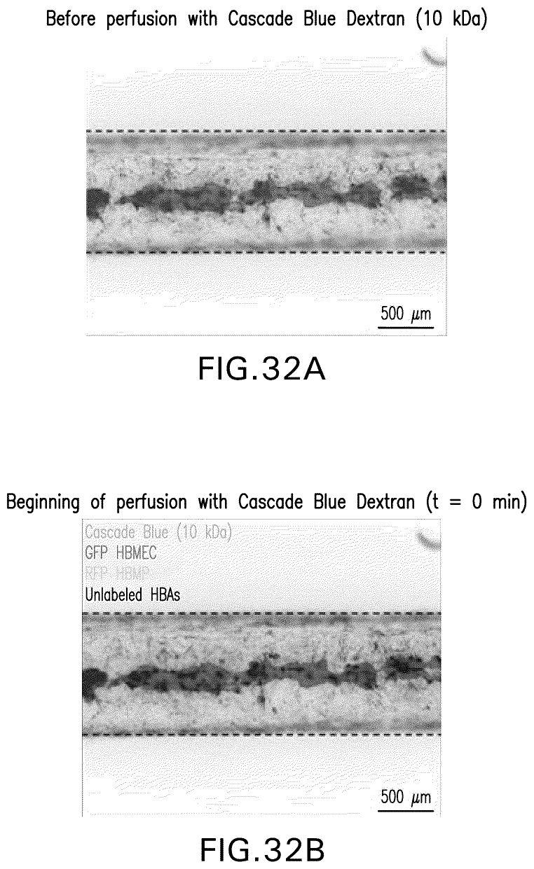

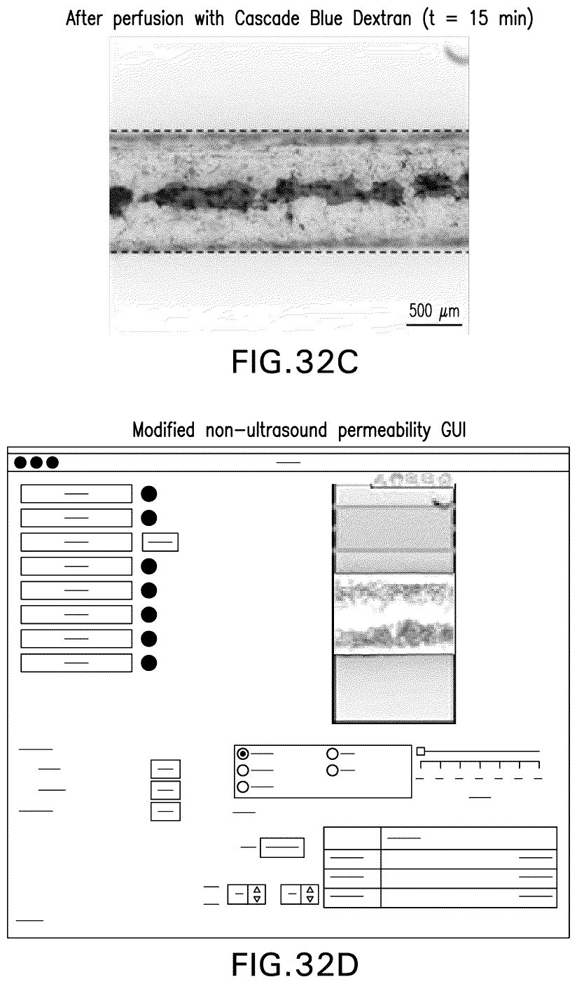

Some of the work of this example is demonstrating pilot studies towards assessing functionality of the tri-culture BBB model. In addition to validating the presence of key cellular junctions and transporters, vascular permeability studies are often conducted to demonstrate sufficient barrier function of the tri-culture model. The framework for these studies was outlined using perfusion of a 10 kDa Cascade Blue Dextran (FIG. 32). In this, 10 kDa Cascade Blue dextran was perfused through the tri-culture channel and the fluorescence intensity of the molecular tracer was assessed outside the channel after a 15-minute permeability trial. To do this, the ultrasound-permeability GUI was developed for use in calculating vascular permeability in the absence of ultrasound. Cascade blue dextran was utilized to mitigate challenges associated with the overlap of fluorophores. This framework will enable quantification of vascular permeability using varying molecular weight dextrans, as well as extension to therapeutically relevant molecules, such as Immunoglobulin G (IgG).

Demonstration of Therapeutic Screening Applications under Ultrasound Application

In some embodiments, the work sought to combine the use of projection stereolithography and endothelialization techniques to seed cells into hydrogels with hollow vascular networks, the workflow developed to quantify vascular permeability and use of ultrasound to enhance transport across the engineered endothelium, and the assembled BBB in vitro model developed as disclosed here.

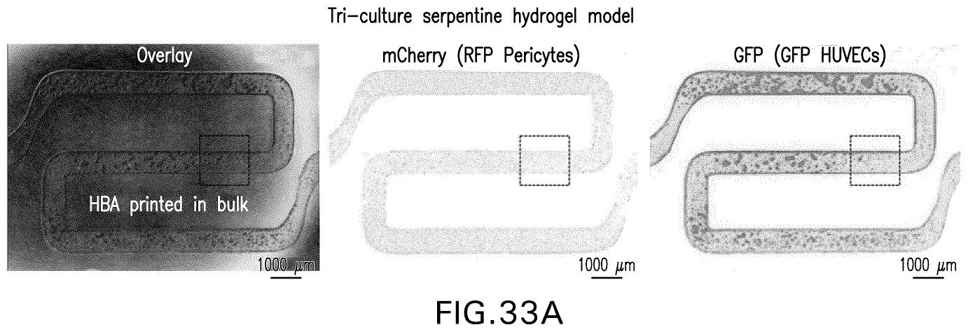

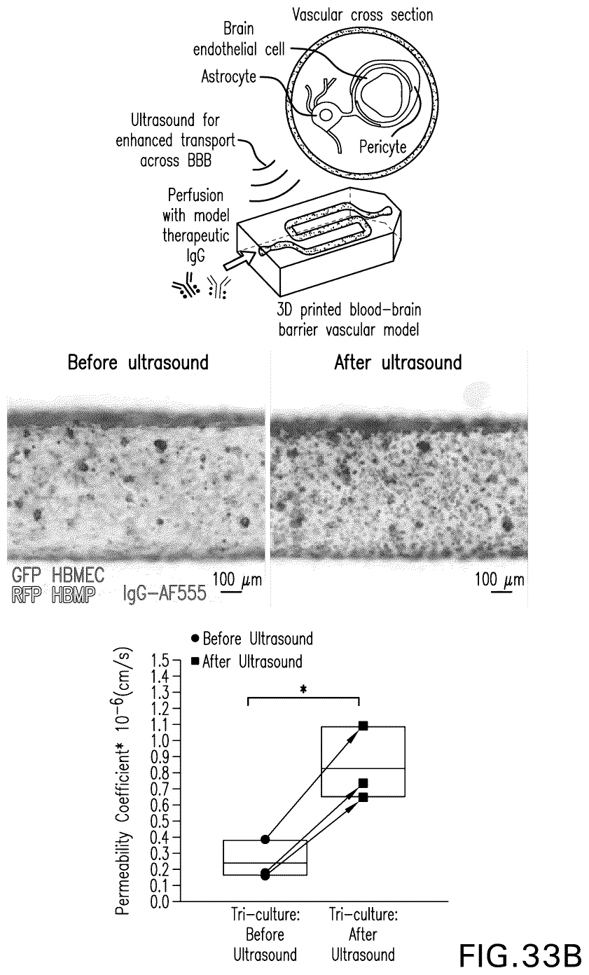

In pilot studies, the fabrication of the BBB tri-culture model in the serpentine architecture (FIG. 33a) was demonstrated and the transport of a clinically relevant compound perfused through the vasculature under the application of ultrasound was assessed (FIG. 33b, left). To represent therapeutic antibodies, a rapidly growing new class of drugs, fluorescently conjugated Immunoglobulin G (IgG) was leveraged in these studies. The perfusate comprising IgG-AlexaFluor-555 and microbubbles to serve as the molecular tracer and agent for sonoporation, respectively, was perfused through the channel and ultrasound was applied. Timelapse acquisitions during the ultrasound-permeability trial enabled assessment of cell morphology and rearrangement during ultrasound. Before ultrasound, a substantial number of endothelial cells as well as pericytes were present in the channel and the endothelial cells appeared to be interdigitating. However, as ultrasound was applied, the cells began to rearrange and shift (FIG. 33b, middle images). An ultrasound-permeability GUI was developed and used to quantify vascular permeability before and after ultrasound application in this tri-culture model, and there was a substantial increase in the vascular permeability coefficient after ultrasound application (FIG. 33b, right). The average permeability of the tri-culture gels before ultrasound was 0.24e-6 cm s⁻¹, while the average permeability of the tri-culture gels after ultrasound was 0.83e-6 cm s⁻¹, demonstrating that the use of ultrasound and microbubbles played a role in enhancing transport of the conjugated antibody across the engineered BBB of this model.

While the tri-culture model displays promise to be used as a tool in assessing the transport of various compounds across the BBB model under ultrasound application, the next steps would be to run the necessary controls with this model such as an astrocyte only bulk gel to assess permeability of the hydrogel without the pericyte and endothelial linings, as well as an astrocyte printed gel with an endothelial only lining to assess the necessity of all three cell types in the model. Overall, this model shows exciting promise as a platform that could be used in the late stages of drug discovery or the early stages of preclinical drug development for assessing the ability of compounds to traverse the BBB in treatment of neurological conditions.

This work has established a 3D printable hydrogel BBB model with relevant cell populations and organization and has demonstrated the potential for evaluating transport across this restrictive barrier in vitro. The 2.5D static hydrogel half pipe provided an efficient method to determine tri-culture parameters in a resource-conservative and high-throughput manner. The translation of these parameters to a 3D perfusable model enables the study of transport in a more physiologically relevant setting under controlled flow conditions. In contrast to existing assays, this model provides the benefit of enabling direct cell-cell contact between endothelial cells and the supporting cell populations of the BBB, a crucial aspect in recapitulating the native environment. Additionally, the use of 3D printing may enable the precise patterning of complex architectures and the ability to pattern regions for the incorporation of additional cell types (cancer cell lines, disease model organoids, etc.) that would enable the fabrication of a more complex disease model where the treatment of diseased cells outside the BBB vasculature could be examined. The confirmation of efflux transporters (P-gp and BCRP) as well as receptor-mediated transporters (TfR and low-density lipoprotein receptor related protein-1) demonstrates that this model is suitable for assessing therapeutic drug delivery strategies whose mechanisms rely on these transporters such as efflux pump inhibition and therapeutic delivery via receptor-mediated transporters. Thus far, this 3D printable BBB model has demonstrated exciting potential for use as a scalable screening platform to investigate transport across this restrictive barrier and has the potential to inform decisions about neurotherapeutic drug design and therapeutic strategies targeting the BBB.

### Example 2: Characterization of Vascular Permeability and Extension to Ultrasound-Assisted Transport through the Cellularized Barrier

Once the necessary cell types have been incorporated for fabrication of the in vitro blood brain barrier model, there are several ways that are used to determine whether the model successfully replicates the in vivo environment by displaying physiological functions before utility for screening applications. For models where it is feasible, one common method to assess barrier integrity is the measurement of transendothelial electrical resistance (TEER). This is a non-invasive way to quantify barrier integrity and is one of the standard methods in the field. TEER is generally used with 2D Transwell models. Additionally, organ-on-a-chip microfluidic devices are suitable for integrating sensors that enable the measurements of TEER in real time. While TEER is compatible with these two common in vitro modelling strategies, the measurement of TEER is difficult in other types of devices due to technical complications associated with incorporating the electrodes.

Another method that is used for demonstrating physiological function in BBB models is the demonstration of cell-cell junctions and transporter protein expression, which can be validated by confocal microscopy of immunocytochemistry, immunohistochemistry, or quantitative real-time polymerase chain reaction (qPCR). The cell-cell junctions most commonly assessed in BBB models are ZO-1, claudin-5, and occluding (tight junctions), as well as VE-Cadherin, CD31, and beta-catenin. The transporters most frequently assessed are P-gp (efflux transporter), and glucose transporter 1, multidrug resistance protein 1, breast cancer resistance protein, and low-density lipoprotein receptor related protein 1. The work outlined in this example demonstrated the presence of some of these cellular junctions and transporter systems, demonstrating functionality of the in vitro model through one of the most common characterization techniques.

Finally, another method used to demonstrate physiological function of the in vitro BBB model is accomplished by quantifying permeability with a fluorescent compound. Generally, different molecular weights of fluorescent dextrans are used to quantify the rate at which the fluorescent molecule crosses from the luminal side of the endothelial cells into the extravascular space. Common molecular weights of dextrans used for characterizing vascular permeability include 4, 10, 70, and 150 kDa. The next steps in this work to demonstrate functionality of the BBB model are to utilize the permeability quantification workflow and quantify permeability of a mono-culture of endothelial cells in the channel, a co-culture of endothelial cells and pericytes in the channel, and finally the tri-culture model of endothelial cells, pericytes, and astrocytes. Permeability of other in vitro BBB models is well-reported in literature, enabling comparison between the current model and others in the field.

Once the function of this model has been sufficiently demonstrated, the model can then be leveraged with a workflow to assess ultrasound-assisted transport through the BBB within this model, as shown in the pilot studies in FIG. 33. Ultrasound-mediated BBB disruption is a technique that has been gaining interest over the last decade as a strategy for delivery of drugs that would normally be impermeable through the BBB. A workflow for quantifying permeability in response to ultrasound was developed, and a perfusable, endothelialized model demonstrated that it was responsive to ultrasound and showed an increase in vascular permeability. That same workflow can be implemented here to assess transport of compounds through the BBB under the application of ultrasound. The workflow developed quantifies permeability of the molecule by using a fluorescent tracer and quantifying the intensity of that tracer outside the channel over time. While this enables a robust workflow for permeability calculation, it should be considered in the design of future experiments. If the compound to be delivered through the vasculature is not fluorescently labeled and cannot be fluorescently tagged, other strategies may be employed to quantify vascular permeability and transport though the vasculature.

One method that could be employed to enable measurement of transport is the use of label-free mass spectrometry. Label-free protein quantification methods are used to determine and quantify relative amounts of proteins in two or more biological samples. This could be used to assess the transport of protein therapeutics perfused through the lumen that are transported through the cellular barrier and into the bulk surrounding gel. In some examples, this method involves protein extraction from the hydrogel bulk gel for processing and may benefit from determination and optimization of digestion methods to extract proteins from the hydrogels for quantification.

### Example 3: Adapting a Stage Top Incubator to Enable Longitudinal Perfusion Studies and BBB Cell-Cell Interactions

Additionally, this in vitro BBB model has the potential to be used in studying the development and maintenance of the BBB by performing longitudinal studies under perfusion culture and assessing cell-cell interactions the formation and stabilization of the BBB. Traditionally, when assessing cell behavior closely, daily imaging occurs to pinpoint morphology changes to a specific timeline. However, a continuous imaging system suitable for perfusion in conjunction with strategically selected cell lines has the potential to enable unique insights to barrier formation. For example, in fabrication of the tri-culture, swapping the GFP-labeled HBMEC for VE-Cadherin-GFP human brain microvascular endothelial cells (VE-Cadherin-GFP-HBMEC), a commercially available cell line that allows for longitudinal assessment of VE-Cadherin development in brain endothelial cells, with longitudinal imaging would inform about the formation of endothelial cell-cell adhesion junctions under flow and in the presence of BBB supporting cells.

To lay the foundation for this work, an ibidi Stage Top Incubation System (Cat.No: 10720) was purchased with the ability for static culture live cell imaging. This product includes the ibidi Heating System (lid set to 40° C.; base set to 37° C.) and the ibidi Gas Incubation System (actively mixed 5% CO₂ at a flow rate of 10 l/hour and 90% humidity). The lid was modified to accommodate inlet and outlet ports (2 each) for fluid perfusion by drilling holes (4×) and inserting male to male luer adapters then scaling with epoxy. The plate insert that holds the glass slide was replaced with a custom 3D printed slide holder that permitted space for catheters to connect into the hydrogel and exit out the sides of the chamber. Additionally, 90-degree needle tips were used to reduce the footprint of the catheter needles within the incubator. Finally, our 3D printed perfusion chambers were adjusted to fit the width of a standard glass slide as well as fit two chambers lengthwise per glass slide to enable the simultaneous perfusion and live imaging of two endothelialized hydrogels. For the perfusion setup, a syringe pump was connected via tubing to the inlet port of the incubator while the outlet port led to a waste container.

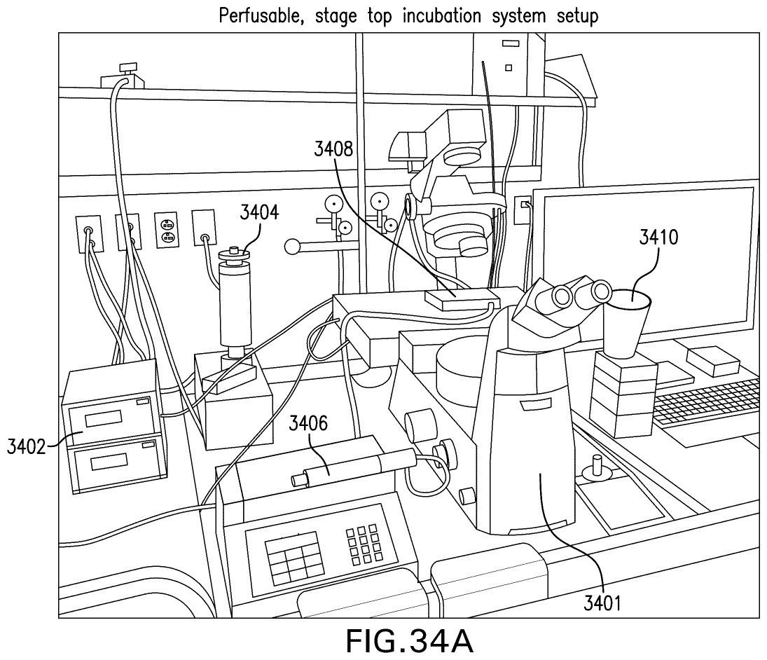

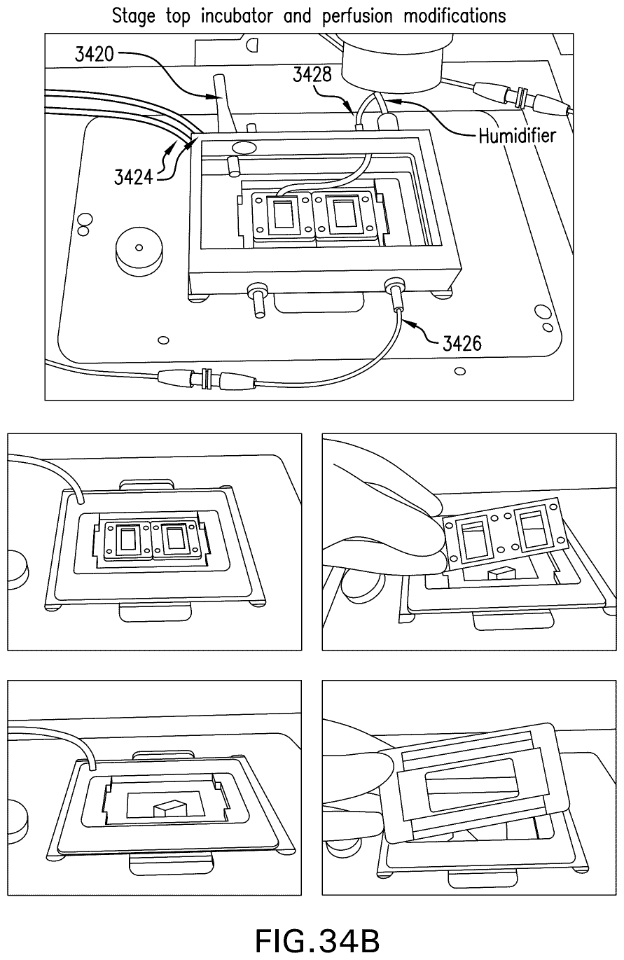

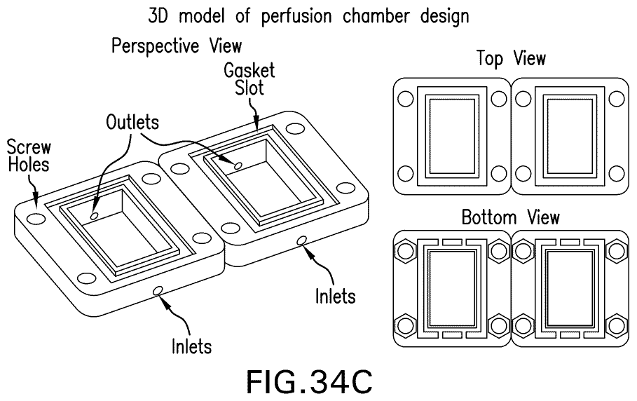

Next, this work aimed to produce a perfusable incubation system that enables longitudinal imaging of endothelialized 3D vascular hydrogel models on an epifluorescent microscope 3401. As a starting point, an ibidi Stage Top Incubation System was purchased and then modified to accommodate our current perfusion chambers, including the addition of inlet and outlet ports to maintain a closed system during perfusion culture. The complete setup, demonstrated in FIG. 34a involves the ibidi temperature and gas control 3402, humidifier 3404, syringe pump 3406, stage top incubator 3408 and waste collection container 3410. The ibidi system controls all aspects of incubation, such as carbon dioxide, oxygen, humidity, and temperature. The humidity is actively monitored by a sensor 3420 inside the chamber lid (FIG. 34b) while the gas is mixed and regulated within the control boxes. The temperature can be set separately for the lid and the base of the incubative chamber.

Multiple customizations may be utilized to incorporate perfusion into this traditionally static system, but it will be understood that any of the disclosed changes are merely exemplary. First, the lid 3424 was modified, as shown in FIG. 34b, to accommodate inlet port 3426 and outlet port 3428 (2 each) for fluid perfusion by drilling holes (4×) and inserting male to male luer adapters then sealing with epoxy. These adapters allow for the system to be used with or without perfusion by simply capping the inlet/outlet ports when not in use. Next, a custom plate insert was designed, and 3D printed in PLA that fits a standard glass slide. This modified slide holder has chamfered sides to allow room for catheters to be inserted into the perfusion chamber and connect the vascular hydrogels to fluidic perfusion. Additionally, 90-degree needle tips were used instead of traditional straight tips to reduce the footprint of the catheter needles within the incubation chamber. Lastly, the design of our perfusion chambers was minimized to fit the width of a standard glass slide (FIG. 33c) while maintaining the same hydrogel size capacity of our current vascular models. To increase throughput, this system was designed to enable live cell imaging on two perfusion-cultured tissues at a time to compare multiple conditions, such as media types, cell types, hydrogel materials, vascular architectures, and more.

A challenge in these longitudinal perfusion studies will be minimizing leakage at all fluidic connections, especially at the insertion point of the catheter needles into the vascularized hydrogel. This risk may be reduced by casting PDMS around the needle tips of the inlet and outlet to act as gaskets. This has tremendously decreased the frequency of leaks in our perfusion models; however, occasional instances (˜<10%) have still occurred in which fluid leaks from the perfusion chamber rather than flowing out the outlet catheter as desired. If this were to happen while a longitudinal acquisition was in progress, the fluid (i.e., media) would drip onto the nosepiece of the microscope and potentially reach the internal components and cause serious damage to the equipment. To reduce this risk, all gels will be perfused for at least 24 hours within the perfusion incubation system on the lab bench off to the side of the microscope to ensure proper fluidic connection prior to moving the system onto the scope for imaging. Furthermore, the system will be monitored for at least 1 hour before leaving unattended and checked frequently for any potential leaks. Alternatively, a catch basin could be designed to fit under the perfusion chamber to capture any unwanted fluids during the perfusion acquisition. The challenge with a catch basin would be having a small enough footprint as to avoid hitting any objectives in use and not obstructing the imaging view in any way. With these challenges in mind, the first experiments should be brief ˜4-6 hour acquisitions that will be monitored continuously to eliminate the risk of potential fluidic damage to the microscope. This longitudinal imaging system may play an important role in understanding endothelial monolayer development in the presence of pericytes and astrocytes under perfusion conditions.

### Example 4: Architectural Freedoms Enabled by 3D Printing Allow for Studies Examining Complex Biological Questions

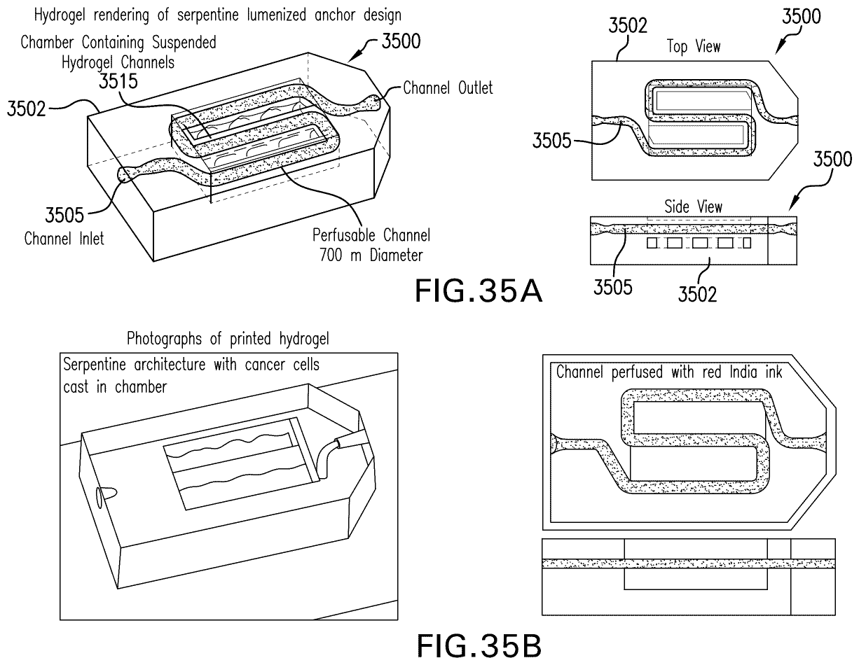

One of the benefits of using 3D printing for the fabrication of an in vitro BBB cell model is the ability to selectively pattern architectures that enable the placement of additional cell types. One example of an architecture that includes this feature, seen below in FIG. 35a, which shows a device 3500 having a hydrogel 3502 that defines a hollow serpentine vascular channel 3505 supported by hydrogel posts underneath and suspended in an open chamber space 3515. In some examples, hydrogel 3502 is approximately 4 mm in height, 10 mm in width and 16 mm in length and may define a serpentine channel 3505 with a 700 μm diameter. Channel 2905 may be spaced from the from the base of hydrogel 3505 as best seen in the side view of FIG. 35a. It will be understood that slight deviations in the channel diameter are possible. The open chamber space 3515 may provide the opportunity to cast additional cells of interest into the chamber which fully encompasses the vascular channel 3505. FIG. 35b shows an example where a cancer cell line is cast into the chamber space (opaque region) and India ink is perfused through the channel for visualization. This architectural design could enable the study of the interactions between diseased cells and the vasculature. For utilizing the BBB model outlined in this example, this model design could be used to study the delivery of treatments to neurological disease model cell lines cast in the surrounding chamber. For example, the BBB model could be fabricated, then a human glioblastoma cell line (T98G or U87MG) could be cast in the surrounding space. With this model fabricated, it could be used to assess delivery of treatments through the vasculature to the surrounding diseased cells. Additionally, in the area of cancer research, this model could be used to study cancer metastasis to the brain via incorporating cancer cells into the perfusate and observing their propensity to travel through the vasculature and interact with the BBB. This could be extended to other neurological disease as well via the incorporation of cerebral organoids modelling Parkinson's or Alzheimer's into the chamber space. This 3D printed model provides the architectural freedom to enable the study of complex interactions between a selected disease model and the vasculature, as well as providing a high-throughput testing platform for screening therapeutic drugs for different neurological conditions.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

What is claimed is:

1. A device for use as a blood-brain barrier model, comprising:
a hydrogel component defining a channel formed between a first opening and a second opening of the hydrogel component, wherein the hydrogel component comprises astrocytes;
pericytes disposed in the channel; and
endothelial cells disposed in the channel.

2. The device of claim 1, wherein the pericytes disposed in the channel adhere to the hydrogel component.

3. The device of claim 2, wherein the pericytes adhere to the hydrogel component via the astrocytes.

4. The device of claim 1, wherein the endothelial cells disposed in the channel adhere to the hydrogel component.

5. The device of claim 4, wherein the endothelial cells adhere to the hydrogel component via the astrocytes.

6. The device of claim 1, wherein the pericytes are in direct contact with the endothelial cells.

7. The device of claim 6, wherein the pericytes are in direct contact with the endothelial cells on a side of the endothelial cells that faces the hydrogel component.

8. The device of claim 1, wherein the astrocytes are human brain astrocytes.

9. The device of claim 1, wherein the pericytes are human brain microvascular pericytes.

10. The device of claim 1, wherein the endothelial cells are human brain microvascular endothelial cells.

11. The device of claim 1, wherein the hydrogel component further comprises poly(ethylene glycol) diacrylate (PEGDA).

12. The device of claim 1, wherein the hydrogel component further comprises lithium phenyl-2,4,6-trimethylbenzoylphosphinate (LAP).

13. The device of claim 1, wherein the hydrogel component further comprises gelatin methacrylate (GelMA).

14. The device of claim 1, wherein the channel has a diameter of at least 200 micrometers and at most 2 millimeters.

15. The device of claim 1, further comprising a housing chamber enclosing at least a portion of the hydrogel component, wherein the housing chamber has a first port aligned with the first opening and has a second port aligned with the second opening.

16. The device of claim 15, wherein the housing chamber is made from a plastic.

17. The device of claim 1, wherein the endothelial cells express one or more blood-brain barrier transporters.

18. The device of claim 17, wherein the one or more blood-brain barrier transporters comprise p-glycoprotein 1 efflux pump, breast cancer resistance protein efflux pump, transferrin receptor, or low-density lipoprotein receptor-related protein 1.

19. The device of claim 18, wherein the one or more blood-brain barrier transporters comprise p-glycoprotein 1 efflux pump, breast cancer resistance protein efflux pump, transferrin receptor, and low-density lipoprotein receptor-related protein 1.

20. A device for use as a blood-brain barrier model, comprising
a hydrogel component defining a groove, wherein the hydrogel component comprises astrocytes;
pericytes disposed in the groove; and
endothelial cells disposed in the groove.

21. The device of claim 20, wherein the pericytes disposed in the groove adhere to the hydrogel component.

22. The device of claim 21, wherein the pericytes adhere to the hydrogel component via the astrocytes.

23. A method of selecting a neurotherapeutic compound, comprising
introducing a plurality of neurotherapeutic compounds, either separately or collectively, to the channel of one or more of the devices of any one of claim 1;
determining the relative permeabilities of the plurality of neurotherapeutic compounds into the hydrogel component; and
selecting at least one neurotherapeutic compound based on its determined relative permeability.
